(19) United States
(12) Patent Application Publication
(10) Pub. No.: US 2026/0108289 A1
(43) Pub. Date: Apr. 23, 2026

(54) PULSE FIELD ABLATOR

(71) Applicant: QIL Engineering Ltd., Yokneam (IL)

(72) Inventors: Yosef HAZAN, Haifa (IL); Meir ATALI, Ramat Yishai (IL); Eliran BENISTI, Neot Golan (IL); Hen VOLCHNOCK, Poriya Ilit (IL)

(73) Assignee: QIL Engineering Ltd., Yokneam (IL)

(21) Appl. No.: 19/428,598

(22) Filed: Dec. 22, 2025

Related U.S. Application Data
(63) Continuation of application No. PCT/IL2024/050599, filed on Jun. 19, 2024.
(60) Provisional application No. 63/521,887, filed on Jun. 20, 2023.
(60) Provisional application No. 63/551,103, filed on Feb. 8, 2024.

(51) Int. Cl. A61B 18/00; A61B 17/00

(52) U.S. Cl. CPC A61B 18/00; A61B 2017/00137; A61B 2017/00867; A61B 2018/00065; A61B 2018/00077; A61B 2018/00083; A61B 2018/00148; A61B 2018/00178; A61B 2018/00184; A61B 2018/0022; A61B 2018/00267; A61B 2018/00351; A61B 2018/00577; A61B 2018/00732; A61B 2018/00761; A61B 2018/00767

(57) ABSTRACT

An ablation catheter device, including: an elongated flexible body having a long axis, a proximal end and a distal end; an expandable therapeutic portion coupled to the elongated flexible body and located between the proximal end and the distal end of the elongated flexible body, wherein the expandable therapeutic portion comprises at least one central flexible shaft configured to move between a collapsed state and an expanded state; and two or more elongated electrodes coupled to the expandable therapeutic portion, wherein the two or more elongated electrodes are twisted around the at least one central flexible shaft.

### RELATED APPLICATIONS

This application is a Continuation of PCT Patent Application No. PCT/IL2024/050599 having International filing date of Jun. 19, 2024 which claims the benefit of priority under 35 USC § 119(e) of U.S. Provisional Patent Application Nos. 63/521,887 filed on Jun. 20, 2023 and 63/551,103 filed on Feb. 8, 2024. The contents of the above applications are all incorporated by reference as if fully set forth herein in their entirety.

### FIELD AND BACKGROUND OF THE INVENTION

The present invention, in some embodiments thereof, relates to ablation of tissue and, more particularly, but not exclusively, to pulse field ablation of tissue.

Background art includes, U.S. Pat. No. 9,387,031; U.S. Patent Application Publication Number 2021/0393327; U.S. Patent Application Publication Number 2022/0104875; U.S. Patent Application Publication Number 2022/0079667; U.S. Pat. Nod. 10,172,673; 11,786,300; U.S. Patent Application Publication Number 2020/0008869; U.S. Patent Application Publication Number 2021/0401490; and U.S. Patent Application Publication Number 2022/0211426.

### SUMMARY OF THE INVENTION

Some exemplary embodiments are listed below (it should be noted that one or more features of some embodiments may be used in combination with one or more features of another embodiment):

According to an aspect of some embodiments there is provided an ablation catheter device, comprising:
an elongated flexible body having a long axis, a proximal end and a distal end;
an expandable therapeutic portion coupled to the elongated flexible body and located between the proximal end and the distal end of the elongated flexible body, wherein the expandable therapeutic portion comprises at least one central flexible shaft configured to move between a collapsed state and an expanded state; and
two or more elongated electrodes coupled to the expandable therapeutic portion, wherein the two or more elongated electrodes are twisted around the at least one central flexible shaft.

According to some embodiments, the at least one central flexible shaft acquires a linear formation aligned with a long axis of the elongated flexible body in the collapsed state, and a lasso-like ring structure in the expanded state, and wherein in the expanded state the two or more elongated electrodes are twisted around the lasso-like ring structure.

According to some embodiments, the two or more elongated electrodes are coated with a dielectric coating.

According to some embodiments, each of the two or more electrodes is formed from at least one wire.

According to some embodiments, the at least one central flexible shaft is formed from an electrically insulating material or is coated with an electrically insulating material.

According to some embodiments, the at least one central flexible shaft is formed from a shape memory alloy.

According to some embodiments, in the collapsed state a maximal width of the therapeutic portion is smaller than 3 mm.

According to some embodiments, the device comprises at least one additional electrode located distally to the therapeutic portion or at a distal end of the elongated flexible body.

According to some embodiments, the device comprises at least one ring electrode coupled to and surrounding the elongated flexible body, wherein the at least one ring electrode is located proximally to the therapeutic portion.

According to an aspect of some embodiments there is provided an ablation catheter device, comprising:
an elongated flexible body having a long axis, a proximal end and a distal end;
an expandable therapeutic portion coupled to the elongated flexible body and located between the proximal end and the distal end of the elongated flexible body, wherein the expandable therapeutic portion is configured to move between a collapsed state and an expanded state; and
two or more elongated electrodes extending axially along the expandable therapeutic portion.

According to some embodiments, a length of each of the two or more elongated electrodes is at least 3 mm.

According to some embodiments, a distance between two adjacent electrodes of the two or more elongated electrodes is at most 7 mm.

According to some embodiments, the expandable therapeutic portion comprises a braided mesh structure configured to move between the collapsed state and the expanded state, and wherein the two or more elongated electrodes surround the braided mesh structure.

According to some embodiments, the two or more elongated electrodes are coupled to the braided mesh structure, or are interwoven within the braided mesh structure.

According to some embodiments, the braided mesh structure is formed from a non-conductive material, or is coated with an electrical insulation material.

According to some embodiments, the two or more elongated electrodes surround entirely the braided mesh structure along a circumference of the braided mesh structure.

According to some embodiments, the two or more elongated electrodes are substantially parallel to each other along the circumference of the braided mesh structure.

According to some embodiments, the two or more elongated electrodes are separated from each other along an axial length of the braided mesh structure.

According to some embodiments, a plurality of the elongated electrodes are independently controllable.

According to some embodiments, the at least one of the elongated electrodes includes an insulated wire that extends proximally from the expandable therapeutic portion.

According to some embodiments, at least a portion of the two or more elongated electrodes includes an electrically isolating material.

According to some embodiments, the two or more elongated electrodes are twisted around or interwoven with the braided mesh structure and/or extend along an axial length of the braided mesh structure.

According to some embodiments, in the expanded state the braided mesh structure acquires a spherical shape.

According to some embodiments, the expandable therapeutic portion comprises at least one central shaft coupled to the braided mesh structure, wherein pushing, pulling and/or rotating of the at least one central shaft moves the braided mesh structure between the collapsed state and the expanded state.

According to some embodiments, the braided mesh structure is formed from a polymer or from a shape memory alloy.

According to some embodiments, in an expanded state a cross section of the braided mesh structure is circular.

According to some embodiments, the two or more electrodes have a helical, zigzag or a straight line pattern around the braided mesh structure.

According to some embodiments, the two or more electrodes include a plurality of first electrodes having a first polarity and a plurality of second electrodes having a second polarity;
wherein the body distal end includes a conductive distal plate; and
wherein the plurality of first electrodes are electrically connected to the conductive distal plate and the plurality of second electrodes are electrically isolated from the conductive distal plate.

According to some embodiments, the two or more electrodes include a plurality of first electrodes having a first polarity and a plurality of second electrodes having a second polarity;
wherein the body proximal end includes a conductive proximal plate; and
wherein the plurality of second electrodes are electrically connected to the conductive proximal plate and the plurality of first electrodes are electrically isolated from the conductive proximal plate, wherein one of the first electrodes extends proximally through an aperture in the conductive proximal plate and is connected to a conductive wire that extends proximally toward the body proximal end, and wherein one of the second electrodes extends proximally through the conductive proximal plate and is connected to the conductive wire.

According to some embodiments, the expandable therapeutic portion comprises an enclosed inflatable structure configured to move between a deflated state in the collapsed state, and an inflated expanded state in the expanded state, and wherein the two or more elongated electrodes are coupled to an outer surface of the enclosed inflatable structure and surround the enclosed inflatable structure.

According to some embodiments, the two or more electrodes are axially spaced apart on the enclosed inflatable structure.

According to some embodiments, the two or more electrodes are parallel to each other on the outer surface of the enclosed inflatable structure.

According to some embodiments, each of the two or more electrodes forms a zigzag pattern around the enclosed inflatable structure.

According to some embodiments, the therapeutic portion comprises segments of electrically insulating coating partially covering the electrodes and the outer surface of the enclosed inflatable structure while keeping exposed sections shaped as rings surrounding the therapeutic portion that include exposed portions of adjacent electrodes.

According to some embodiments, the enclosed inflatable structure comprises at least one balloon.

According to some embodiments, the enclosed inflatable structure is formed from an electrically insulating polymer.

According to some embodiments, the device comprises at least one additional electrode coupled to the elongated flexible body and located distally to the therapeutic portion.

According to some embodiments, the device comprises at least one ring electrode coupled to the elongated flexible body and located proximally to the therapeutic portion.

According to some embodiments, the expandable therapeutic portion includes a plurality of axially extending wires extending distally from the body when in the collapsed state, the plurality of axially extending wires including at least a first electrode wire extending axially from the body and having a first polarity and at least a second electrode wire extending axially from the body and having a second polarity, wherein at least the first electrode wire is separated from the second electrode wire by a preselected distance.

According to some embodiments, the plurality of axially extending wires are formed at least in part of a shape memory alloy material, the expandable therapeutic portion configured to expand from a first, collapsed configuration to a second, expanded configuration.

According to some embodiments, the expandable therapeutic portion includes a plurality of axially extending electrodes each having a distal portion extending circumferentially around the expandable therapeutic portion.

According to some embodiments, the electrode distal portions are substantially parallel to each other.

According to some embodiments, the expandable therapeutic portion includes from four to ten axially extending electrodes.

According to some embodiments, the device includes at least one tensioner for at least one of:
maintaining the elongated electrodes at a preselected distance apart from each other;
maintaining the elongated electrodes at a preselected distance away from a longitudinal axis of the expandable therapeutic portion; and
preventing inadvertent deformation of the elongated electrodes.

According to some embodiments, the expandable therapeutic portion, when in the expanded state, is configured as a cage having generally vertical bars.

According to some embodiments, each the elongated electrode includes a distal end portion extending radially inwardly and proximally.

According to some embodiments, the plurality of axially extending wires each includes a proximal wire portion and a distal wire portion; and
wherein at least one of the proximal wire portions and the distal wire portions includes an isolating material configured to maintain adjacent ones of the plurality of axially extending wires at a preselected distance apart from each other.

According to some embodiments, the isolating material is one of:
a coating on the plurality of axially extending wires;
a plating on the plurality of axially extending wires;
a cover radially external to the plurality of axially extending wires; and
a first cover radially external to the plurality of axially extending wires and a second cover radially internal to the plurality of axially extending wires.

According to some embodiments, the isolating material is one of:
a cover radially external to the plurality of axially extending wires, the cover including through holes; and
a first cover radially external to the plurality of axially extending wires and a second cover radially internal to the plurality of axially extending wires, wherein the first and second cover include through holes.

According to some embodiments, the isolating material is at least one of biocompatible, non-thrombogenic, and microporous.

According to some embodiments, the isolating material is selected from enamel, ePTFE (polytetrafluoroethylene), and polyimide.

According to some embodiments, the axially extending wires include the isolating material on at most 15% of a most distal portion thereof.

According to some embodiments, the axially extending wires include the isolating material on at most 50% of a most proximal portion thereof.

According to some embodiments, the two or more elongated electrodes include a nitinol core plated or coated with a conductive material.

According to some embodiments, the conductive material is selected from gold, a platinum alloy, a palladium alloy, and any combination thereof.

According to an aspect of some embodiments there is provided an ablation catheter device, comprising:
an elongated flexible body having a long axis, a proximal end and a distal end;
an expandable therapeutic portion coupled to the elongated flexible body and located between the proximal end and the distal end of the elongated flexible body, wherein the expandable therapeutic portion is configured to move between a collapsed state and an expanded state; and
first and second electrode sections surrounding the expandable therapeutic portion, wherein the first and second electrode sections have respective first and second polarities, the first and second electrode sections separated by an insulative section.

According to some embodiments, the first electrode section is positioned on a proximal portion of the expandable therapeutic portion and the second electrode section is positioned on a distal portion of the expandable therapeutic portion.

According to some embodiments, the first electrode section is larger than the second electrode section.

According to some embodiments, the insulative section forms an undulating curve around the expandable therapeutic portion.

According to some embodiments, the insulative section forms a zigzag-shaped border or a border having undulations with each of the first and second electrode sections.

According to some embodiments, each of the first and second electrode sections includes projections extending toward the insulative section.

According to some embodiments, the projections each have a same width.

According to some embodiments, each of the first and second electrode sections and the insulative section extends spirally about the expandable therapeutic portion.

According to some embodiments, the first electrode section includes a first portion extending circumferentially around the expandable therapeutic portion and a second portion extending from the body to the first portion; and
wherein the second electrode section includes a third portion extending circumferentially around the expandable therapeutic portion and a fourth portion extending distally from the third portion.

According to some embodiments, the device includes a radiopaque marker on at least one of the first and second electrode sections.

According to some embodiments, the insulative portion extends circumferentially around the expandable therapeutic portion.

According to some embodiments, the insulative portion is positioned along a circumferential portion of the expandable therapeutic portion, perpendicular to the body longitudinal axis.

According to some embodiments, the ablation portion is configured to ablate tissue having a width corresponding to a distance between the first and second electrode sections.

According to an aspect of some embodiments there is provided an ablation catheter device, comprising:
an elongated flexible body having a long axis, a proximal end and a distal end;
an expandable therapeutic portion coupled to the elongated flexible body and located between the proximal end and the distal end of the elongated flexible body, wherein the expandable therapeutic portion is configured to move between a collapsed state and an expanded state; and
first and second electrode sections surrounding the expandable therapeutic portion, wherein the first and second electrode sections have respective first and second polarities, the first and second electrode sections separated by an insulative section;
wherein the insulative section includes an undulating area extending circumferentially around the expandable therapeutic portion.

According to an aspect of some embodiments there is provided a system for ablating tissue comprising:
an elongated ablation device comprising:
an expandable therapeutic portion configured to move between a collapsed state and an expanded state; and
two or more elongated electrodes surrounding the expandable ablation portion;
a control unit coupled to the elongated ablation device, comprising:
a memory configured for storing parameter values of an electric field suitable for delivery of pulse field ablation (PFA) to a body tissue, wherein the parameter values comprise at least one of voltage, frequency, pulse duration and number of pulses;
a pulse generator electrically connected to the two or more electrodes;
a control circuitry, wherein the control circuitry is configured to signal the pulse generator to generate the electric field according to the parameter values stored in the memory and to deliver the generated electric field to the two or more electrodes.

According to some embodiments, the voltage is between 300 volts and 3000 volts.

According to some embodiments, the frequency is between 150 kHz and 1000 kHz.

According to some embodiments, the pulse duration is between 0.05 ms and 500 ms.

According to some embodiments, the expandable therapeutic portion comprises a braided mesh structure configured to move between a collapsed state and an expanded state, and wherein the two or more elongated electrodes surround the mesh structure.

According to some embodiments, the two or more electrodes extend along an axial length of the braided mesh structure.

According to some embodiments, the two or more electrodes are located at different axial locations along a length of the braided mesh structure.

According to some embodiments, the two or more electrodes are twisted around or interwoven with the mesh structure.

According to some embodiments, the control circuitry is configured to signal the pulse generator to generate and deliver an electric field via at least one electrode of the two or more electrodes to a cardiac tissue with parameter values suitable for pacing the tissue.

According to some embodiments, the elongated ablation device comprises at least one additional electrode located distally to the expandable therapeutic portion, and wherein the control circuitry is configured to signal the pulse generator to generate and deliver an electric field via the at least one additional electrode to a tissue with parameter values suitable for pacing the tissue.

According to some embodiments, the two or more elongated electrodes comprise at least 4 elongated electrodes and wherein the control circuitry is configured to select one or more pairs of electrodes out of the at least 4 elongated electrodes and to signal the pulse generator to generate and deliver the electric field with the parameter values via the one or more selected pairs of electrodes to a body tissue.

According to some embodiments, the control unit comprises a user interface configured to receive input signals from a user of the system and to deliver human detectable indications to the user, and wherein the control circuitry selects the one or more pairs of electrodes based on input signals received from the user via the user interface.

According to an aspect of some embodiments there is provided an ablation catheter device, comprising:
an elongated flexible body having a long axis, a proximal end and a distal end;
a therapeutic portion coupled to the elongated flexible body and located between the proximal end and the distal end of the elongated flexible body, wherein the therapeutic portion includes an axially extending portion;
wherein the therapeutic portion includes at least first and second electrodes having respective first and second polarities;
wherein the therapeutic portion is configured to move between a collapsed state, in which the axially extending portion is positioned along the long axis, and an expanded state in which the axially extending portion assumes a spiral shape.

According to some embodiments, the at least first and second electrodes are wound around the axially extending portion and wherein the at least first and second electrodes are evenly spaced from each other.

According to some embodiments, the axially extending portion is defined by the first and second electrodes.

According to some embodiments, the axially extending portion is defined by the first electrode, wherein the second electrode is wound around the first electrode and is maintained at a distance from the first electrode.

According to some embodiments, a first portion of the axially extending portion is configured to contact tissue to be ablated and wherein the first portion of the axially extending portion is defined by the first and second electrodes; and
wherein a second portion of the axially extending portion is configured to be positioned at a distance from tissue to be ablated.

According to some embodiments, the device includes a dielectric cover configured to prevent or reduce the intensity of an electric field passing from the therapeutic portion to tissue in contact with the therapeutic portion.

According to some embodiments, in the expanded state, the axially extending portion assumes shape having one of: a spiral configuration, a cylindrical configuration, and a zigzag configuration defining a single plane.

According to some embodiments, in the expanded state, the axially extending portion assumes spiral shape having a radius in a range of from 4-18 mm.

According to some embodiments, the axially extending portion has a cross-sectional profile having a diameter in a range of from 1.5-4 mm.

According to some embodiments, the therapeutic portion includes at least a third electrode wound around the axially extending portion and evenly spaced from each of the first and second electrodes.

According to some embodiments, the ablation catheter device is configured to operate in a plurality of ablation modes, wherein in each one of the plurality of ablation modes the ablation catheter device is configured to deliver pulse field ablation (PFA) of a preselected protocol to a body tissue.

According to some embodiments, at least one of:

in a first ablation mode, the device is configured to use the first and second electrodes to deliver the PFA;

in a second ablation mode, the device is configured to use the first and third electrodes to deliver the PFA; and in a third ablation mode, the device is configured to use the second and third electrodes to deliver the PFA.

According to some embodiments, the therapeutic portion includes at least a fourth electrode wound around the axially extending portion and evenly spaced from each of the first, second, and third electrodes.

According to some embodiments, each the electrode has a cross-sectional profile having a diameter in a range of from 0.1-0.5 mm.

According to some embodiments, each the electrode has a cross-sectional profile that is circular or elliptical.

According to some embodiments, a distance between adjacent windings of the electrodes is in a range of from 1-5 mm.

According to some embodiments, when in the expanded state, the axially extending portion assumes a spiral shape having a pitch between adjacent spiral rings in a range of from 3-15 mm.

According to some embodiments, when in the expanded state, the axially extending portion assumes a spiral shape having from 0.5 to 5 revolutions.

According to some embodiments, the electrodes are one of:
glued to the axially extending portion;
partially embedded in the axially extending portion.

According to some embodiments, the axially extending portion includes a nitinol wire configured to self-expand to assume a spiral shape.

According to an aspect of some embodiments there is provided a method for delivery of a pulse field ablation (PFA), comprising:
expanding a therapeutic portion of an ablation device having at least two elongated electrodes at least partly within an ostium of a pulmonary vein in a left atrium;
delivering at least one pulse of an electric field between the at least two electrodes and to a wall of the pulmonary vein with ablation parameter values suitable for pulse field ablation; and
generating a non-thermal ablation region in at least one of tissue of the pulmonary vein and heart tissue which surrounds a lumen of the pulmonary vein by the delivered at least one electric field pulse, wherein the non-thermal ablation region prevents transmission of electrical signals in the heart tissue via the non-thermal ablation region.

According to some embodiments, the non-thermal ablation region is substantially uniform and continuous.

According to some embodiments, herein the expanding comprises placing the at least two elongated electrodes in contact with the pulmonary vein wall prior to and during the delivering.

According to some embodiments, the method comprises determining, prior to the delivering, parameter values of the electric field according to at least one of a location of the at least two electrodes within the pulmonary vein ostium, a shape and/or size of a target ablation region, and a target ablation depth, and wherein the delivering comprises delivering the at least one electric field pulse having the determined parameter values.

According to some embodiments, the parameter values comprise intensity, frequency and/or duration of the at least one electric field pulse.

According to some embodiments, the at least two elongated electrodes comprise at least 4 elongated electrodes, and wherein the method comprises selecting one or more pairs of electrodes according to at least one of a location of the at least two electrodes within the pulmonary vein ostium, a shape and/or size of a target ablation region, and a target ablation depth, and wherein the delivering comprises delivering at least one electric field pulse via the selected electrode pairs to the pulmonary vein tissue.

According to some embodiments, the method comprises adjusting, prior to the delivering, a pitch or a distance between the at least two elongated electrodes according to at least one of location of the at least two electrodes within the pulmonary vein ostium, a shape and/or size of a target ablation region, and a target ablation depth.

According to some embodiments, the method comprises pacing, following the delivering, the heart tissue by at least one electrode of the ablation device; and mapping signal transmission via the heart tissue following the pacing and determining a result of the delivering according to the mapping.

According to some embodiments, the ablation device includes a distal ablation portion, and wherein each the elongated electrode includes a proximal portion extending generally parallel to a longitudinal axis of the ablation device and a distal portion extending circumferentially around the ablation portion.

According to some embodiments, the method includes, before the delivering, selecting an ablation mode from a plurality of ablation modes, wherein in each one of the plurality of ablation modes the therapeutic portion is configured to deliver pulse field ablation (PFA) of a preselected protocol to a body tissue.

According to an aspect of some embodiments there is provided a method for delivery of a pulse field ablation (PFA), comprising:
expanding a therapeutic portion of an ablation device at least partly within an ostium of a pulmonary vein in a left atrium, wherein the ablation device includes an ablation portion having two electrode sections separated by an insulative section;
delivering at least one pulse of an electric field between the two electrode sections and to a wall of the pulmonary vein with ablation parameter values suitable for pulse field ablation; and
generating a non-thermal ablation region in at least one of tissue of the pulmonary vein and heart tissue which surrounds a lumen of the pulmonary vein by the delivered at least one electric field pulse, wherein the non-thermal ablation region prevents transmission of electrical signals in the heart tissue via the non-thermal ablation region.

According to an aspect of some embodiments there is provided a method for manufacturing of a therapeutic portion of an ablation device, comprising:
rotating at least one elongated electrode wire, comprising at least one elongated insulation free exposed region, around a central long axis of a base structure; and
twisting the at least one elongated electrode wire around a body of the base structure and along a length of the base structure, while contacting the body and during the rotating.

According to some embodiments, the at least one elongated electrode wire comprises at least two elongated electrode wires positioned at a target distance therebetween, and wherein the twisting comprises twisting the at least two elongated electrode wires while maintaining the target distance therebetween on the base body.

According to some embodiments, the method comprises: determining the target distance prior to the twisting.

According to some embodiments, the method comprises modifying the target distance during the twisting.

According to some embodiments, the base structure comprises at least one electrically insulating flexible wire, and wherein the twisting comprises twisting the at least one elongated electrode wire around the at least one electrically insulating flexible wire to form the therapeutic portion.

According to some embodiments, the rotating comprises rotating at least one electrically insulating wire around the central long axis of the base structure, and wherein the twisting comprises twisting the at least one electrically insulating wire around the base structure body and along a length of the base structure during the rotating.

According to some embodiments, the twisting comprises twisting the at least one electrically insulating wire in synchronization with the twisting of the at least one elongated electrode wire.

According to some embodiments, the at least two elongated wires comprise at least three elongated wires each positioned at a target distance therebetween.

According to some embodiments, the method comprises rotating a first elongated electrode wire, comprising at least one elongated insulation free exposed region, around a central long axis of a base structure; and
twisting at least a second elongated electrode wire around the first elongated electrode wire, while contacting the base structure and during the rotating.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system. ” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining ablation parameters, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a general flow chart of a tissue ablation process, according to some exemplary embodiments;

FIG. 2A is a schematic illustration of a generation of an ablated region in the prior-art;

FIG. 2B is a schematic illustration of a generation of an ablated region according to some exemplary embodiments;

FIGS. 3A and 3B are schematic views of an ablated region, according to some exemplary embodiments;

FIG. 4 is a block diagram of a system and a device, for example a catheter, for ablating tissue, according to some exemplary embodiments;

FIG. 5A is a schematic view of an ablation device having a therapeutic portion, for example an ablation portion, with long electrodes surrounding at least partly the ablation portion, according to some exemplary embodiments;

FIG. 5B is a schematic illustration of an ablated region generated by selection of specific pairs of electrodes of the electrodes of the ablation device shown in FIG. 5A, according to some exemplary embodiments;

FIG. 5C is a schematic illustration of ablated regions, each is generated by selection of specific pairs of electrodes of the electrodes of the ablation device shown in FIG. 5A and using different ablation parameters, according to some exemplary embodiments;

FIG. 6 is a flow chart of an ablation process of heart tissue, according to some exemplary embodiments;

FIGS. 7A-7C are schematic illustrations of an ablation device comprising an expandable ablation portion having long electrodes in a collapsed axial state (7A) and in an expanded state (7B-7C) where the ablation portion expands and acquires a ring lasso-like shape, according to some exemplary embodiments;

FIGS. 7D and 7E are schematic illustrations showing the ablation portion of the device shown in FIG. 7A, expanded within an opening of a blood vessel, for example a pulmonary vein ostium, in the left atrium prior and during an ablation process, according to some exemplary embodiments;

FIGS. 7F and 7G are schematic illustration showing a relation between a distance between two elongated electrodes, for example wire electrodes twisted together around a support structure, for example a base structure, and a depth of an ablation region formed by an electric field delivered to the tissue by the two elongated electrodes, according to some exemplary embodiments;

FIGS. 8A-8C are schematic illustrations of an ablation device comprising an expandable ablation portion formed from a braided mesh having two or more long electrodes twisted around or interwoven with the circumference of the ablation portion and axially extending along a long axis of the ablation portion, according to some exemplary embodiments;

FIG. 8D is a schematic illustration of the ablation device shown in FIGS. 8A-8C with the ablation portion in an expanded state, according to some exemplary embodiments;

FIG. 8E is a schematic illustration showing the ablation portion expanded at least partly within an opening of a blood vessel, for example an opening of a pulmonary vein, in the heart, prior to and during an ablation process, according to some exemplary embodiments;

FIGS. 9A-9D are schematic illustrations of an ablation device comprising an expandable ablation portion formed from a braided mesh having two or more long electrodes axially spaced apart from each other along a length of the ablation portion and surrounding the ablation portion, according to some exemplary embodiments;

FIG. 9E is a schematic illustration showing the ablation portion of the device of FIGS. 9A-9D in an expanded state, according to some exemplary embodiments;

FIG. 9F is a schematic illustration showing the ablation portion of the device of FIGS. 9A-9E in an expanded state, at least partly within an opening of a blood vessel, for example a pulmonary vein in the heart, according to some exemplary embodiments;

FIGS. 10A-10D are schematic illustrations of an ablation device comprising at least one inflatable enclosed scaffold serving as a base for electrodes, according to some exemplary embodiments;

FIG. 10E is a schematic illustration showing the ablation portion of the device of FIGS. 10A-10D in an expanded state, at least partly within an opening of a blood vessel, for example a pulmonary vein in the heart, according to some exemplary embodiments;

FIGS. 11A-11F are results of simulation of a distribution of an electric field in the tissue surrounding an ablation portion, according to some exemplary embodiments FIGS. 12A-12B are schematic illustrations showing manufacturing of a therapeutic portion, for example an ablation portion, of a device, according to some exemplary embodiments;

FIG. 13A is a schematic illustration of an ablation device comprising an expandable ablation portion in a collapsed axial state, according to some exemplary embodiments;

FIG. 13B is an enlarged schematic illustration of the expandable ablation portion of the device of FIG. 13A;

FIG. 14A is a schematic illustration of the ablation device of FIG. 13A, in an expanded state; FIG. 14B is an enlarged schematic illustration of the ablation portion of the device of FIG. 14A;

FIG. 14C is a schematic illustration showing the ablation device of FIG. 14A, expanded within an opening of a pulmonary vein ostium, according to some exemplary embodiments;

FIGS. 15A-C are schematic illustrations of expandable ablation portions of ablation devices according to alternative exemplary embodiments;

FIG. 15D is a schematic illustration showing the ablation device of FIG. 15C, expanded within an opening of a pulmonary vein ostium, according to some exemplary embodiments;

FIGS. 16A-B are schematic illustrations of expandable ablation portions of FIG. 14B, including at least one additional tensioning wire, according to some exemplary embodiments;

FIGS. 16C-D are schematic illustrations of expandable ablation portions of ablation devices, according to some exemplary embodiments;

FIGS. 16E-G are schematic illustrations of portions of an expandable ablation device, according to some exemplary embodiments;

FIG. 17A is a schematic illustration showing an ablation device in an expanded state, according to some exemplary embodiments;

FIG. 17B is a schematic illustration showing an enlargement of the ablation portion of the device of FIG. 17A in an expanded state, according to some exemplary embodiments;

FIG. 17C is a schematic illustration showing the ablation device of FIG. 17A in an expanded state, at least partly within an opening of a pulmonary vein in the heart, according to some exemplary embodiments;

FIGS. 18A-E are schematic illustrations showing the ablation portions of alternative ablation devices, each in an expanded state, according to some exemplary embodiments;

FIG. 19A is a schematic illustration showing an ablation device in an expanded state, according to some exemplary embodiments;

FIG. 19B is a schematic illustration showing the ablation portion of the device of FIG. 19A in an expanded state, according to some exemplary embodiments;

FIG. 19C is a schematic illustration showing the ablation portion of an alternative ablation device in an expanded state, according to some exemplary embodiments;

FIG. 20A is a schematic illustration showing an ablation device having a spiral ablation portion according to some exemplary embodiments; and

FIGS. 20B-C are schematic illustrations showing the ablation portion of FIG. 20A, wherein a portion of the device has been removed (in FIG. 20B) to show features thereof.

### DESCRIPTION OF SPECIFIC EMBODIMENTS OF THE INVENTION

The present invention, in some embodiments thereof, relates to ablation of tissue and, more particularly, but not exclusively, to pulse field ablation of tissue.

### Overview

An aspect of some embodiments relates to generating at least one long continuous ablated region in a tissue by delivery of at least one pulse of an electric field to the tissue. In some embodiments, the at least one electric field pulse is delivered to the tissue via at least two elongated electrodes contacting the tissue, optionally a surface of the tissue. In some embodiments, the ablated region has a long dimension, for example a length, of at least 10 mm, for example at least 15 mm, at least 20 mm, at least 25 mm, at least 30 mm, or ant intermediate, smaller or larger range of values. In some embodiments, the long dimension of the ablated region extends along a circumference or a portion thereof, of a blood vessel wall or a body cavity wall contacted by the at least two elongated electrodes.

According to some embodiments, a depth of the ablated region in the tissue, for example a distance from a contact location of the electrode with the tissue and into the tissue, varies less than 10%, less than 5%, less than 1%, or any intermediate, smaller or larger value, along a long dimension, for example length, of the ablated region.

According to some embodiments, the ablation is a non-thermal ablation of the tissue. In some embodiments, the ablation is a pulse field ablation (PFA) of the tissue. In some embodiments, the tissue is a tissue of the heart. In some embodiments, the tissue ablation comprises electroporation of the tissue, optionally irreversible electroporation of the tissue.

According to some embodiments, the ablation comprises ablating heart tissue to interfere with unwanted electrical transmission via heart tissue, and/or to ablate one or more regions in a heart tissue that pace the heart. In some embodiments, the ablation is delivered via elongated electrodes of the ablation device contacting a wall of a pulmonary vein (PV) of the heart, optionally a wall of a pulmonary vein funnel adjacent a left atrium of the heart, i.e., a wall of a pulmonary vein having a funnel-like configuration, entering into the atrial cavity, for example, a wall of a pulmonary vein expanding more than about 10% of the average diameter of the pulmonary vein further away from the left atrium, or a wall of a pulmonary vein extending about 10-30 mm further distally away from the left atrium. In some embodiments, a tissue contact region with the elongated electrodes surrounds at least 30% of a circumference of the blood vessel, for example the PV, for example surrounds at least 50%, at least 70%, at least 80%, at least 90, at least 95%, or any intermediate, smaller or larger percentage of the circumference of the blood vessel. According to some embodiments, this may be potentially advantageous in that the less than 100% contact may be required with the tissue region to be ablated, while still providing 100% ablation. In some embodiments, the ablation, for example the PFA ablation, is performed in a subject diagnosed with atrial fibrillation. In some embodiments, at least one electrode of the ablation device, for example at least one electrode at a distal end or edge of the device, is used to perform point ablation.

According to an aspect of some exemplary embodiments, an ablation device includes an ablation portion which is, optionally, axially extending. The ablation portion may have a structure which may be in a collapsed state, for introduction into a body, via a catheter or other delivery device, to a location near tissue to be ablated. The structure may assume an expanded state, when released from the delivery device and may be flexible when in the expanded state. The flexibility of the structure when in the expanded state may allow contact with and conforming tissue to be ablated such as, for example, an entire circumference within a pulmonary vein funnel. According to some embodiments, the ablation portion is flexible enough such that it will not cause damage to or deform tissue in which it comes into contact for performing the ablation procedure. The ablation portion may include electrodes or electrodes, at least one of which has a first polarity and at least one of which has a second polarity.

According to some embodiments, the structure itself may be formed of a plurality of electrode wires, optionally configured as a braided mesh structure, at least some of which electrode wires may contact and/or conform to tissue to be ablated. The structure may include a first electrode wire having a first polarity and a second electrode wire having a second polarity, the first and second electrodes utilized for performing an ablation procedure. According to some embodiments, all of the electrode wires or portions of all of the electrode wires, may contact the tissue to be ablated and may be employed in the ablation procedure.

According to some embodiments, when in an expanded state, a first portion of the structure may be configured to contact and/or conform to tissue to be ablated and may include at least first and second electrodes having respective first and second polarities, the first and second electrodes to be utilized for performing an ablation procedure. A second portion of the structure may not contact the tissue but, rather, may be positioned at a distance from the tissue to be ablated.

According to some embodiments, when released from the delivery device, the structure may assume an expanded state, and a plurality of electrode wires, including a first electrode having a first polarity and a second electrode having a second polarity, may be wound around the structure, such that the plurality of electrode wires may contact and/or conform to the tissue to be ablated and may be employed for performing an ablation procedure.

According to some embodiments, at least some electrodes of the mesh structure may each have its own wire so that the electrodes may be controlled separately. For example, there may be six separate electrodes interwoven in the braid of the mesh structure, each electrode having its own insulated wire that extends proximally within a shaft, so that any pair of electrodes from the six electrodes may be selected to provide ablation, depending on the area to be ablated.

According to some embodiments, preselected portions of any of the electrodes may be provided with an electrically isolating material such as, for example, an enamel, ePTFE (polytetrafluoroethylene), polyimide, FEP (fluorinated ethylene propylene), PFA (perfluoroalkoxy alkanes), or any other insulative/isolating material discussed herein, or any other suitable material that may insulate one or more portions of the electrodes. This allows ablation of a preselected portion of tissue, depending on the portion(s) of the electrodes not provided with isolating material. According to some embodiments, when released from the delivery device, the structure may assume an expanded state. The structure itself may define a first electrode having a first polarity, and at least one electrode wire having a second polarity may be wound around the structure, such that an ablation procedure may be performed utilizing the first electrode and the at least one electrode wire.

According to some embodiments, the at least two elongated electrodes are located on a circumference of a therapeutic portion, for example an ablation portion, for example an expandable ablation portion, configured to move between a collapsed state and an expanded state. In some embodiments, the electrodes are twisted around and/or extend around the circumference of a therapeutic portion. In some embodiments, the electrodes are twisted around as they extend around the circumference of a therapeutic portion. In some embodiments, the electrodes, optionally formed from electrically conducting wires or stripes surround at least partly or entirely the ablation portion. Additionally, in some embodiments the electrodes extend along an axial length of the ablation portion. In some embodiments, the electrodes extend along of at least 5 mm, for example at least 10 mm, at least 15 mm, at least 20 mm, at least 25 or any intermediate smaller or larger value. Alternatively, each electrode is located at a different axial position along the axial length of the ablation portion.

According to some embodiments, the electrodes are separated by an electrically non-conducting region of the ablation portion. In some embodiments, the electrodes are substantially parallel to each other, with a deviation of less than 10 mm, for example less than 5 mm, less than 3 mm, less than 1 mm, less than 0.5 mm, or any intermediate, smaller or larger value in a distance between two adjacent long electrodes of an ablation portion. In some embodiments, a distance between two adjacent electrodes is in a range between about 0.5 mm and 50 mm, for example in a range between about 0.5 mm and 1.5 mm, in a range between about 1 mm and 10 mm, in a range between about 5 mm and 30 mm, in a range between about 20 mm and 50 mm, or any intermediate, smaller or larger value or range of values.

An aspect of some embodiments relates to an ablation device wherein the ablation portion comprising the long electrodes is an expandable ablation portion configured to expand, for example reversibly expand, for example to place the long electrodes in contact with a surface of the tissue. In some embodiments, the expandable ablation portion comprises an expandable mesh structure, for example braided mesh structure. In some embodiments, the ablation portion, for example the mesh structure is expanded to acquire the shape of a lasso, stent, braided, mesh, or a cage having generally vertical bars, or any other flexible shape that will hold two or more long electrodes with a circular/oval/foil profile shape, small in diameter and in certain distance or pitch to ensure better compliance and adaptation between the electrodes and heart anatomy and/or a desired ablation depth in the heart tissue.

During manufacture of the braided mesh structure of the ablation device, for example, electrodes may be interwoven into an elongated braided sleeve and the sleeve may be cut into preselected lengths. This may reduce manufacturing costs. Additionally, the provision of an elongated braided mesh having axially extending electrodes interwoven therethrough may facilitate preparation of the ablation device, for example, for a PFA procedure, as the operator does not have to manually position the electrodes along the mesh structure. According to some embodiments, in order to maintain a specific distance between the electrodes fixed on the ablation portion and optionally avoid sharp edges, a shaft or a structure of a mesh forming the ablation portion comprises dedicate twisted grooves which are shaped and sized to hold the electrodes in place. In some embodiments, the grooves position is preformed according to at least one of, a planned position of the electrodes, a target pattern of the electrodes, and/or a target, for example a desired distance between the electrodes.

An aspect of some embodiments relates to an ablation device wherein an ablation portion may include a plurality of axially extending electrode wires, wherein the ablation portion may include from four to ten axially-extending electrode wires, such as, for example, eight electrode wires. Optionally, the electrode wires are formed of a shape memory alloy material such as, for example, nitinol, which allows the ablation portion to expand from a collapsed configuration to an expanded configuration in or near a tissue to be ablated such as, for example, a blood vessel. The electrode wires may include at least a first electrode wire having a first polarity and at least a second electrode wire having a second polarity, such as, for example, a first, positive electrode wire and a second, negative electrode wire, where the electrode wires do not intersect and a distance between positive and negative electrode wires is maintained, according to some embodiments. A potential advantage of an ablation portion having electrode wires and non-electrode wires such as, for example, electrically isolating wires, that do not intersect is that the electrode wires may better conform to the shape of tissue to be ablated, optionally becoming embedded in the tissue when it is pressed thereagainst. Additionally, an ablation portion having electrodes wires which do not intersect each other may be more flexible and/or more compressible than, for example, an ablation portion formed of a mesh.

An aspect of some embodiments relates to an ablation device including an ablation portion which may be provided with a tensioning structure, optionally, one or more tensioning wires for maintaining the electrode wires at a preselected distance apart and/or for maintaining wires at a preselected distance away from a longitudinal axis of the ablation portion and/or for preventing inadvertent deformation of the electrode wires, according to some embodiments. An aspect of some embodiments relates to an ablation device including an insulating material coated or plated onto proximal and/or distal portions of the electrode wires. Alternatively, the insulating material may be in the form of a cover positioned radially outside the ablation portion or both radially outside and radially inside the ablation portion. Optionally, the cover may include apertures for allowing blood to flow therethrough. The insulating material or cover may be biocompatible, non-thrombogenic, and/or microporous. The insulating material or cover may ensure that portions of electrode wires that are to contact tissue to be ablated, i.e., portion of the electrode wires that are not covered by insulating material, are maintained equidistant from each other.

An aspect of some embodiments relates to an ablation device wherein an ablation portion may include an inflatable balloon or other selectively inflatable portion having formed thereon portions having predefined shapes which are coated, electroplated, metalized, or printed on sections of the balloon surface with electrically conductive material which may be resistant to abrasion. Resistance to abrasion may be important so that the balloon may be inserted into a delivery sheath at the beginning of the procedure and may be retracted back into the delivery sheath when necessary, without damaging the electrically conductive material. Additionally, as the electrically conductive material may be resistant to abrasion, its performance during the procedure will not be affected in any way, and no residue will be left inside the patient or inside the delivery sheath.

The electrically conductive sections on the balloon which are coated, electroplated, metalized, or printed during manufacture may provide electrodes having specific patterns on the balloon, and a wired ring may be positioned at each of a proximal portion and a distal portion of the balloon, to provide connections between the electrode sections and a body of the ablation device, as discussed herein. This may reduce manufacturing costs, as electrodes do not need to be added onto the balloon by the operator.

Optionally, the balloon may include portions which are folded, when in the collapsed state, which unfold so that the balloon can assume the inflated state. According to some embodiments, the ablation portion may include a first electrode or electrode section having a first polarity and a second electrode or electrode section having a second polarity such as, for example, a first, positive electrode or electrode section and a second, negative electrode or electrode section, where an insulated or non-conductive area is disposed between the electrodes or electrode sections having different polarity. The ablation portion may expand from a collapsed state to an expanded state by application of energy, and an electrical field may be generated between the electrodes of different polarities, thereby allowing ablation of tissue such as, for example, heart tissue, located between the electrodes of different polarities, according to some embodiments.

Prior to being expanded, the ablation device may be deployed from within a left atrium, at least partly within an opening of a pulmonary vein in the heart, according to some exemplary embodiments, according to some embodiments.

Optionally, the ablation portion may optionally include at least one radiopaque marker in the form of at least one radiopaque band, optionally on at least one of the electrode having a first polarity and the electrode having a second polarity such as, for example, a first, positive electrode and a second, negative electrode. The provision of at least one radiopaque marker may assist an electrophysiologist, for example, to visualize the location of the electrode or electrode section under x-ray, according to some embodiments. This may allow the approximation of what tissue will be ablated. Further, it should be appreciated by person skilled in the art that, optionally, any of the embodiments shown and/or discussed herein may be provided with radiopaque marker, as discussed herein, according to some embodiments.

Shapes of the first electrode or electrode section having a first polarity, the second electrode or electrode section having a second polarity, and insulative portion therebetween may be preselected, according to the shape of tissue it is desired to ablate, according to some embodiments. For example, the first electrode or electrode section having a first polarity, the second electrode or electrode section having a second polarity, and the insulative portion therebetween may define a shape having a straight line, a wavy line; a zigzag, i.e., a pattern made of connected segments that repeatedly change directions at angles smaller than 150 degrees; a spiral; or any other desired shape, extending around at least a part of the ablation portion, according to some embodiments. The balloon may be non-complaint, half compliant or fully complaint, according to some embodiments. Optionally, the inflatable portion may contact a large area within the funnel of a pulmonary vein or inside a pulmonary vein.

An aspect of some embodiments relates to an ablation portion which may include an inflatable balloon having formed thereon a first electrode or electrode section having a first polarity and a second electrode or electrode section having a second polarity, the first and second electrode sections having predefined shapes, as discussed herein, wherein the non-conductive area disposed between the electrode sections may define a shape having border defined by wavy lines or undulations, according to some embodiments. This particular configuration may provide for a larger ablation area, due to the larger border between electrode sections.

According to some embodiments, the ablation portion can be preformed to be expanded or shrink to a certain dimension in order to fit at least partly inside a selected or a specific body cavity, for example into the pulmonary vein or any section in the left atrium, and create full circle ablation (no gap between first and last electrode).

According to some embodiments, each electrode of the device or the ablation portion is connected to a wire that passes along the catheter shaft up to a proximal section of the catheter, optionally configured to be located outside the patient body. In some embodiments, the electrodes are configured to transfer voltage in a range between about 300 volts and 3,000 volts, for example voltage between about 300 volts and 800 volts, voltage between about 500 volts and 1000 volts, voltage between about 800 volts and 2000 volts, voltage between about 1000 volts and 3000 volts, or any intermediate, smaller or larger range of values. In some embodiments, when the electrodes, for example the long electrodes are in contact with the tissue, for example heart tissue, an electric field is generated and is delivered between the electrodes and into the heart tissues to create a linear or a patterned ablation region between them, based on irreversible electroporation (IRE) which forms a massive nanoscale membrane-permeable holes on the cell's membrane in the target tissues.

According to some embodiments, two or more electrodes of the ablation portion are selected according to a desired shape and/or size of a planned ablated region. In some embodiments, when using more than two electrodes, an electrical connection to the electrodes is switched for example to generate electric field between electrodes that are not adjacent to each other to increase the electric field and optionally create a deeper ablation region. Optionally, one or more electrodes that are not used for ablation are used as sensing electrodes to receive feedback on the electric field delivered to the tissue. Alternatively or additionally, a pitch distance of the electrodes during the ablation procedure is changed to increase or reduce the electric field between the electrodes.

According to some embodiments, the ablation device, for example the ablation portion comprises a tip or a distal end, located distally to the ablation portion that comprises one or more additional electrodes. In some embodiments, the one or more additional electrodes are used to perform point ablation of the tissue. Alternatively, the one or more additional electrodes are used for pacing the tissue. Optionally, long electrodes of the ablation portion extend to the distal end or tip, and can be used as the one or more additional electrodes for performing point ablation of the tissue and/or for paving.

According to some embodiments, the electrodes, for example long electrodes of the ablation portion or one or more additional electrodes of the ablation device, are covered or coated with a dielectric and/or a biocompatible coating, for to avoid sharp edges and blood clots that could be create in one or more gaps, grooves, voids, slots between the electrodes and a shaft or any other region of the ablation portion or the ablation device.

According to some embodiments, the design of the ablation device, for example the ablation catheter allows for example to produce a low-cost catheter with advantages that can be used for delivery of PFA to a tissue, for example heart tissue that will benefit with the advantages of the pulse field ablation technology.

A potential advantage of the device design may be that it provides a unique form of flexibility with better compliance between the catheter and an anatomy of a body cavity, for example the anatomy the pulmonary vein (PV). For example, due to its small size, a tip of the catheter could enter a conventional in size PV and even a PV that is smaller than normal or a PV having stenosis following a previous ablation procedure.

According to some embodiments, features of the device include using a different pitch or selecting electrodes of the ablation portion in order, for example to generate an ablation region with a desired shape, size and/or depth. In some embodiments, one or more of the electrodes is used to provide feedback of the electric field during procedure. For example, when paring electrodes 1 and 2 of an ablation portion, for example adjacent electrodes, a depth and the width of the ablation area is in a range between about 1-4 mm, for example between about 1-3 mm, about 2-4 mm, or any intermediate, smaller or larger range of values. In some embodiments, when pairing or using electrodes 1-4, a depth and/or a width of the ablation region is larger, for example in a range between about 4.5-9 mm, for example about 4.5-6 mm, about 5-7 mm, about 5-8 mm, or any intermediate, smaller or larger range of values.

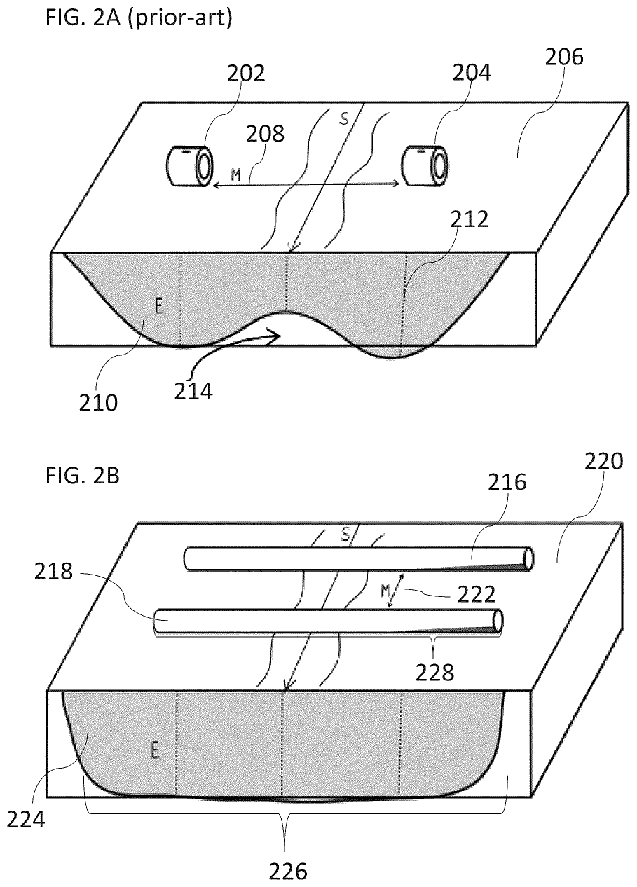

A potential advantage of using long electrodes, for example continuous linear electrodes, may be that the electrical field generated inside the tissue is substantially uniform along the ablation line and closes a full circle of ablation. This is an advantage over using distributed electrodes in the prior-art, for example as shown in FIG. 2A that generates a variable electrical field along the ablation line. This advantage will reduce the need for several ablation repetitions at different catheter orientation.

According to some embodiments, the ablation device, for example the ablation catheter, has a maximal width in a collapsed state of up to 3 mm (9 French) and is flexible, which allows to insert a tip of the catheter into a PV and to expand the ablation portion of the catheter at least partly inside the PV. A potential advantage of expanding the ablation portion of the catheter at least partly within the PV may be to allow performing of an ablation simultaneously from within the PV and at the entrance of the PV, without a need to reposition and repeat the ablation process.

According to some embodiments, the ablation system is configured to change a distance and/or between the electrodes on demand, optionally during a PFA procedure, for example to change a depth of the electric field and ablation. In some embodiments, the electrodes are twisted in a certain pitch/distance from each other that will preformed a certain ablation depth. In some embodiments, during a procedure the E. P (electrophysiologist) can optionally change a construction of a pitch/distance of the electrodes, for example to increase or decrease an ablation depth. Alternatively, for example when using a plurality of electrodes, at least one pair of electrodes is selected, for example to reach a smaller or larger ablation depth.

According to some embodiments, the ablation device comprises at least one pacemaker electrode. In some embodiments, the pacemaker electrode is an additional distal electrode that can potentially replace the need of pacemaker catheter during an ablation procedure. In some embodiments, the electrode is placed inside the PV to stimulate the heart to create specific heart beat signals that can be received by a mapping catheter.

According to some embodiments, the two or more elongated electrodes are twisted around an expandable therapeutic portion, for example an ablation portion, of the ablation device. A potential advantage of using twisted electrodes may be to allow flexibility to use fewer components (number of electrodes) and still get an ablation region with a target size and/or area. In some embodiments, the ablation device can be designed such that the electrodes has a specific pitch relative to each other, and optionally bring the electrodes closer or moving further from each other while still remaining in the same effective area without adding another electrode or changing a length of the ablation device.

According to some embodiments, the ablation device comprise one or more additional electrodes at a distal tip of the device. In some embodiments, the one or more additional electrodes are used for point ablation of the tissue.

An aspect of some embodiments relates to an ablation device having an ablation portion including two or more elongated electrodes located on a lasso-like ring structure or on a stent structure in a straight parallel conformation, or twisted around a flexible shaft, for example a flexible wire expandable to form the lasso-like ring structure.

According to some embodiments, the two or more elongated electrodes are linear continues electrodes, which allow for example to generate a linear ablation region with a substantially uniform depth. The two or more elongated electrodes may include a spline scaffolding having wrapped therearound two elongated electrodes which are sufficient, for example, to provide PFA to a body tissue, thereby potentially reducing time and complexity of assembly of the ablation device which may reduce manufacturing costs.

In some embodiments, since a distance between the electrodes is in a similar direction to a planned insulation line in the heart tissue, the same electrodes can optionally be used to assess a completion of the ablation line by delivery of stimulation via one electrode and receive (or not receive) a signal in the other electrode. In some embodiments, by demonstrating that the other electrode is not receiving the stimulated signal it is possible to determine a completion of the insulation line. This will optionally save the need to use an additional catheter to induce the stimulation signal inside the pulmonary vein.

According to some embodiments, the two or more elongated electrodes have a circular or oval cross section with a small diameter in a range of from about 0.05 to 1.5 mm, for example, from about 0.05-0.06 mm, from about 0.06-0.08 mm, from about 0.08-0.1 mm, from about 0.1 mm - 0.5 mm, from about 0.4 mm-1 mm, from about 0.7 mm-1.5 mm or any intermediate, smaller or larger range of values. Alternatively, the elongated electrodes may each have a generally rectangular or square cross-sectional profile with rounded corners. A potential advantage of using electrodes with a small diameter or width may be to allow a safer interface with the heart tissues without the need to remove sharp edges with glue or other material. Elongated electrodes as discussed herein may be employed in any of the embodiments discussed herein that include elongated electrode wires.

According to some embodiments, the ablation device and system allows a diastole ablation which is shorter than a diastole time (Diastole >0.5 sec.). In some embodiments, the pulse field ablation time is smaller than the diastole period, for example to allow performing the ablation in a stable heart during relaxation. In some embodiments, the PFA ablation pulse is synchronized with an electrocardiogram (ECG) signal and is optionally shorter than 500 milliseconds, and is repeated during the next diastole per the clinical protocol, that can be acquired for the ablation catheter or other catheters used during the procedure.

A potential advantage of performing all the ablation cycles simultaneously may be to improve the efficacy of the ablation procedure and the overall patient outcome and to shorten the ablation process.

A potential advantage of performing PFA ablation may be a reduced risk for generating microbubble due to ablation technology that avoid heating the tissue and the blood in the area of ablation. Since the ablation device contains continues linear electrodes and requires less ablation cycles it may reduce a future risk for generation of microbubbles and therefore increases ablation procedure efficacy and safety.

According to some embodiments, a diameter of the ablation device, for example the ablation catheter is similar to a size of other diagnostic catheters for example 8.5 F catheters, and can be used with a conventional 8.5 F sheath without removing and inserting larger sheath during procedure. Entering the left atrium (LA) with a bigger sheath, might bear a risk for air embolism, which might have been the cause of observed transient ST-elevation and concomitant AV-block. According to some embodiments, a distance between the elongated electrodes is very small e.g., 1 mm between electrodes, allow us to use lower ablation electrical field that allow better control on the overall ablation tissue volume (ablation width and ablation depth control). Lower ablation volume while keeping the required tissue ablation depth, minimize the risk for stenosis increment.

According to some embodiments, the ablation device comprises elongated electrodes with different diameters. Since a wall thickness can vary between different sections of the LA, for example, the wall thickness inside the PV could be thinner than a wall thickness of the PV ostium, it is possible to use electrodes with a smaller diameter on a distal end of the ablation portion, and electrodes with a larger diameter on the proximal portion of the ablation portion. This will allow or example to ensure that the ablation with the smaller electrode will not exceed the necessary depth and will prevent unnecessary damage to the inside part of the PV. For example, a distal portion: ablation <2 [mm], proximal portion: ablation >2 [mm].

According to some exemplary embodiments, a width or a diameter of an elongated electrode is between about 0.1 mm-1.5 mm, for example about 0.1 mm-0.5 mm, about 0.4 mm - 1 mm, about 0.7 mm-1.5 mm or any intermediate, smaller or larger range of values. In some embodiments, a pitch, for example a distance, between adjacent electrodes is between about 0.5 mm-1.5 mm, for example between about 0.5 mm and 1 mm, between about 0.7 mm and 1.2 mm, between about 1 mm and 1.5 mm, or any intermediate, smaller or larger range of values. In some embodiments, a pitch between electrodes affect an ablation depth. For example a pitch between electrodes of about 1 mm, is used to reach about 3 mm deep ablation at 2,000 volts. Alternatively, when using the same electrodes at about 500 volts and about a 2 mm pitch, the ablation depth is about 1 mm, and when increasing the pitch to about 5 mm, optionally by selecting at least one different electrode, the ablation depth can reach about 2.5 mm. In some embodiments, a thickness, for example diameter or width, of the electrodes allows optimization of the electrode's pitch such that the electrical filed will be drastically reduced beyond the depth of ablation

According to some embodiments, the ablation device uses a universal connector with adaptors to different types of generators.

An aspect of some embodiments relates to manufacturing of a therapeutic portion, for example an ablation portion, of an ablation device by twisting at least two elongated wire electrodes around and along a base structure, for example an elongated base structure. In some embodiments, a distance, also termed herein as a “pitch”, between the at least two elongated electrode wires is determined prior to the twisting. Optionally, the determined distance is modified during the twisting. In some embodiments, the at least two elongated electrode wires are twisted around and along the base structure during a rotation of the base structure around a central long axis of the base structure, optionally while a source of the at least two elongated electrode wires is stationary. Alternatively, the at least two elongated electrode wires are twisted around and along the base structure while moving around the base structure. Optionally, the base structure is stationary during the twisting of the electrode wires.

According to some embodiments, the base structure comprises at least one non-electrically conducting wire, for example a flexible wire. In some embodiments, the formed therapeutic portion comprises the at least one electrically insulating wire, and the at least two elongated electrode wires. According to some embodiments, at least one electrically insulating wire, for example a flexible wire, is twisted around and along the base structure, in addition to the at least two elongated electrode wires. In some embodiments, the at least one electrically insulating wire is positioned between the at least two elongated electrode wires, and optionally in a fixed distance from each of the at least two elongated electrode wires. In some embodiments, the at least two elongated electrode wires and at least one electrically insulating wire are twisted around and along the base structure in a first direction, and at least one additional electrically insulating wire is twisted around and along the base structure in a second direction which is opposite to the first direction.

According to some embodiments, an elongated electrode, for example a wire electrode has at least one exposed non-insulated electrically conductive region having a length of at least 10 mm, for example at least 15 mm, at least 20 mm, at least 30 mm, at least 40 mm, at least 50 mm, at least 80 mm or any intermediate, smaller or larger value. In some embodiments, the exposed non-insulated electrically conductive region is configured to be placed at least partly or entirely in contact with a tissue surface, for example to deliver an electric field to the tissue for generating an ablation region in the tissue.

An aspect of some embodiments relates to an ablation device having a flexible ablation portion that, in an expanded state, has a spiral structure. At least two electrode wires, including a first electrode wire having a first polarity and a second electrode wire having a second polarity, may be wound spirally around the ablation portion. The electrode wires may be maintained in position wound around the spiral ablation portion by gluing them to the spiral ablation portion or by partially embedding them in the spiral ablation portion by heating or applying an electric pulse thereto.

The ablation portion, in a collapsed state, may be introduced into a body portion to be ablated such as, for example, a pulmonary vein funnel; the ablation portion may be expanded, as discussed herein; and a pulse field may be produced between the first and second electrode wires to ablate tissue in contact with the ablation portion.

The ablation device having a flexible, spirally-shaped ablation portion has a potential advantage in that it may facilitate ablation of a pulmonary vein funnel, and optionally of a large portion of the funnel, due to its spiral shape, including narrower coils at the distal end of the spirally-shaped ablation portion, and due to the ability of the flexible spirally-shaped ablation portion to conform well to the shape of the funnel, even in an irregularly-shaped funnel.

Additionally, the ablation portion may be provided with at least three electrode wires spirally wound around the spiral structure, thereby providing the ablation device with a plurality of ablation modes, each having a corresponding PFA (pulse field ablation) intensity. The plurality of ablation modes may be preselected, depending on preselected distances between the electrodes that are activated during an ablation procedure.

It should be understood that the term wire or wires may also mean a strand of several wires twisted together, or a strip.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Ablation Process

According to some exemplary embodiments, pulse field ablation (PFA) is performed by delivery of an electric field between two electrodes in a vicinity or contacting tissue surface. In some embodiments, the PFA is a non-thermal ablation, which generates perforations in a cell membrane. In some embodiments, the delivered electric field causes non-thermal, optionally irreversible, electroporation of the cells, for example electroporation of the cell membrane, which leads to death of the cell.

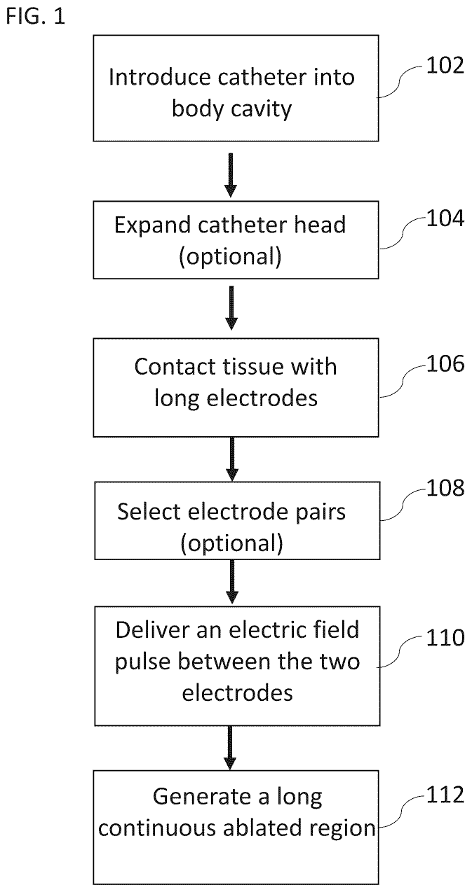

Reference is now made to FIG. 1 depicting a general PFA process, according to an aspect of some exemplary embodiments.

According to some exemplary embodiments, a catheter is introduced into a body cavity, for example a blood vessel, at block 102. In some embodiments, the body cavity is a body lumen. In some embodiments, the body cavity comprises an artery, a vein, a heart ventricle and/or a heart atrium. In some embodiments, the catheter comprises a catheter body having a catheter head, for example an ablation head. In some embodiments, the catheter head is introduced into the body cavity at block 102. Additionally, the catheter head is positioned at a target location within the body cavity, for example a previously selected target for performing PFA. Optionally, the catheter head is an expandable catheter head, configured to move between a collapsed state, for example when the catheter head is introduced into the body cavity, and an expanded state when the catheter head expands to contact walls of the body cavity and/or to anchor the catheter head within the body cavity.

According to some exemplary embodiments, the catheter head is optionally expanded, at block 104. In some embodiments, the catheter head comprises at least one long electrode, for example at least two electrodes, for example at least one electrode pair. In some embodiments, the catheter head is optionally expanded when the at least one electrode is positioned at a target location for performing the PFA. In some embodiments, the catheter head is expanded in response to a force applied on the expandable catheter head from outside the body of the subject, optionally via the catheter body. In some embodiments, the force is a mechanical force, a magnetic force, or an electric force.

According to some exemplary embodiments, tissue is contacted by the expanded catheter head, for example by at the least one long electrode of the catheter head, at block 106. In some embodiments, the at least one electrode, for example the at least one electrode pair, contacts a tissue, for example a surface of the tissue, at block 106. In some embodiments, the tissue is contacted with a force which is sufficient to anchor and/or to stabilize a position and/or an orientation of the catheter head within the body cavity.

According to some exemplary embodiments, one or more electrode pairs is optionally selected at block 108. In some embodiments, one or more electrode pairs is selected according to at least one of, size, length, width, area, and/or volume of a desired, for example a target ablated region. In some embodiments, the one or more electrode pairs are in contact with the tissue, for example with a surface of the tissue.

According to some exemplary embodiments, an electric field is delivered between the at least two electrodes, at block 110. In some embodiments, the at least two electrodes are electrodes of the at least one electrode pair optionally selected at block 108. In some embodiments, the electric field is delivered between the at least two electrodes and into the tissue. In some embodiments, the electric field is delivered with parameter values that are sufficient to generate a target ablation region in the tissue having at least one of, a desired length, a desired depth and/or a desired width. In some embodiments, the electric field parameters comprise at least one of, intensity, frequency, pulse duration, interval between pulses, and overall ablation time of a plurality of pulses delivered to the tissue at the target region.

According to some exemplary embodiments, a long continuous ablated region is generated at block 112. In some embodiments, the long continuous ablated region is generated by the electric field delivered at block 110 between the at least two electrodes.

### Exemplary Ablation Region

Reference is now made to FIG. 2A depicting delivery of an electric field between two points, for example ring, electrodes, as described in the prior-art. As shown in FIG. 2A, two ring electrodes 202 and 204 contact a tissue surface 206 at a distance 208 therebetween. An electric field delivered between the two ring electrodes generates an ablation region 210 with uneven depth 212. For example, as shown in FIG. 2A, a depth of the ablation region 210 is reduced at locations located at a distance from each of the ring electrodes 202 and 204, where the maximal ablation depth is reached closer to the electrodes contact points with the tissue surface 206. The difference in ablation depth along the ablation region generates a void 214 in the ablation region 212 at an axial location between the two electrodes 202 and 204.

Reference is now made to FIG. 2B, depicting delivery of an electric field between two long electrodes, according to some exemplary embodiments.

According to some exemplary embodiments, two long electrodes 216 and 218 are placed in contact with tissue surface 220 and at a distance 222 therebetween. In some embodiments, an electric field delivered between the two long electrodes 216 and 218 generates a continuous long ablation region 224. In some embodiments, a length 226 of the ablation region 224 is at least 20 mm, for example at least 25 mm, at least 40 mm, at least 50 mm, at least 80 mm, at least 100 mm, or any intermediate, smaller or larger value. In some embodiments, a length 228 of each long electrode is at least 10 mm, for example at least 15 mm, at least 20 mm, at least 30 mm, at least 40 mm, at least 50 mm, at least 80 mm or any intermediate, smaller or larger value. In some embodiments, a distance 222 between two adjacent electrodes, for example electrodes 216 and 218 is up to 30 mm, for example up to 20 mm, up to 10 mm, up to 5 mm, up to 1 mm, or any intermediate, smaller or larger value. Optionally, the electrodes contact each other at one or more contact points along their length.

Having a continuous long ablation region allows a faster ablation procedure by reducing the number of ablation repetitions needed when using point electrodes as shown in FIG. 2A, to achieve a target ablation region having a target size and/or depth.

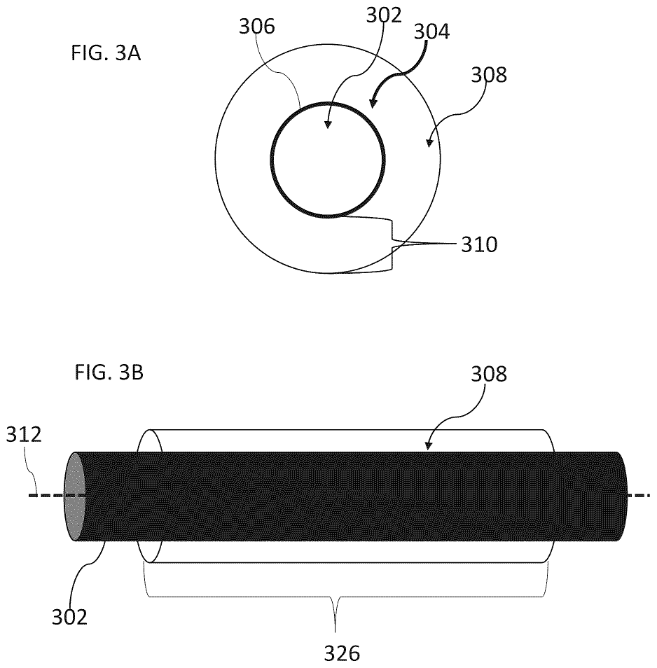

Reference is now made to FIGS. 3A and 3B, depicting a shape and/or size of an ablated region in a tissue surrounding a body cavity, for example a blood vessel, according to some exemplary embodiments.

According to some exemplary embodiments, a PFA ablator is introduced into a lumen 302 of a blood vessel 304. In some embodiments, an electric field is delivered via electrodes of the PFA ablator contacting a wall 306 of the blood vessel 304. In some embodiments, the delivered electric field generates an ablated region 308 in the tissue that surrounds at least partly or entirely the blood vessel 304. In some embodiments, the ablated region 308 is shaped as an arc subtended by an angle having a degree between about 1 degree and about 360 degrees, for example, an angle between about 45 degrees and about 360 degrees, an angle between about 90 degrees and about 360 degrees, an angle between about 150 degrees and about 210 degrees, an angle between about 180 degrees and about 360 degrees or an angle in any intermediate, smaller or larger range of degrees. Alternatively, the ablated region is shaped as a ring, for example, along wall 306 in FIG. 3A.

According to some exemplary embodiments, a thickness 310 of the ablated region 308, for example a distance between the blood vessel wall and an end of the ablated region, is within a range between about 0 mm and about 10 mm, for example in a range between about 0.1 mm and about 3 mm, in a range between about 0.5 mm and about 5 mm, in a range between about 1 mm and about 7 mm, in a range between about 2 mm and about 10 mm, or any intermediate, smaller or larger range of values. In some embodiments, the ablated region 308 starts from a surface of the tissue and reaches a depth of up to 10 mm from the surface of the tissue.

Additionally, for example as shown in FIG. 3B, the ablated region extends along a long axis 312 of the blood vessel 304. In some embodiments, the ablated region 308 extends along a length of at least 0.1 mm, for example along a length of at least 0.5 mm, at least 1 mm, at least 1.5 mm, at least 3 mm, or any intermediate, smaller or larger value, of the long axis of the blood vessel. In some embodiments, an axial length 326 of the ablated region which is optionally parallel to the long axis 312, is at least 0.1 mm, for example at least 0.5 mm, at least 1 mm, at least 3 mm, at least 5 mm, or any intermediate, smaller or larger value. In some embodiments, the ablation region is a complete, closed and substantially uniform shape along an axial length of the ablated region.

According to some exemplary embodiments, a change in thickness 310 of the ablated region 308 along length 326 is smaller than 25%, for example smaller than 20%, smaller than 15%, smaller than 10%, smaller than 5%, smaller than 1%, smaller than 0.5%, or any intermediate, smaller or larger value.

### Exemplary Ablator Device and System

According to some exemplary embodiments, a system for delivery of PFA comprises an ablator device, for example a PFA ablator, and a control unit. In some embodiments, the control unit controls at least one of, a movement of the ablator device to a target region within a body cavity, anchoring and/or deployment of an ablating portion of the ablator within the body cavity, and/or delivery of an electric field to the tissue. Optionally, the system comprises a mapping unit coupled to the control unit or part of the control unit, for mapping the electrical conductivity of the body cavity tissue before and/or following PFA.

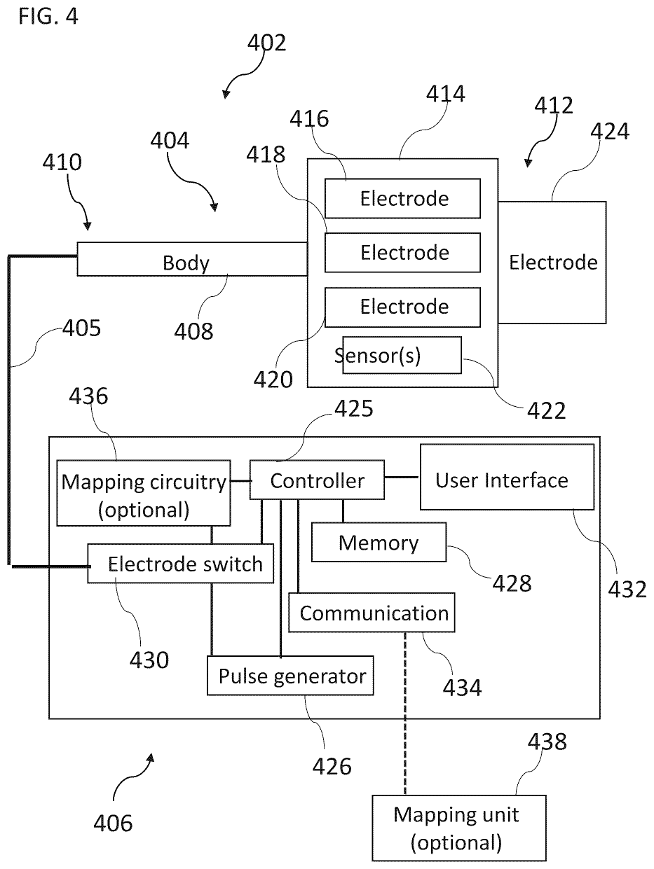

Reference is now made to FIG. 4 depicting an ablation system, for example a PFA ablation system, according to some exemplary embodiments.

According to some exemplary embodiments, a system 402 comprises an ablation device 404, for example an ablation catheter, coupled to a control unit 406. In some embodiments, the device 402 is coupled to the control unit 406 by one or more wires, cables and/or tubes, for example one or more wires 405.

According to some exemplary embodiments, the device 404 comprises a body 408, which is optionally a flexible and/or bendable body, configured to bend and pass within one or more blood vessels towards a target region, for example an ablation target region, within a body cavity. Alternatively or additionally, the body is shaped and sized to pass within a sheath or a tubular body, forming a channel within the body of the subject. In some embodiments, the body 408 is an elongated body having a proximal section 410 located closer to an entrance location into the subject body, and a distal section 412 located closer to an ablation target region within the subject body.

According to some exemplary embodiments, the device 404 comprises an ablation portion 414, for example an ablation head. In some embodiments, the ablation portion 414 is located between the proximal section 410 and the distal section 412 of the device body 408. In some embodiments, the ablation portion 414 is located closer to the distal end of the body 408 or at the distal end of the body 408.

According to some exemplary embodiments, the ablation portion 414 comprises two or more electrodes, for example electrodes 416, 418 and 420. In some embodiments, at least one electrode of the electrodes 416, 418 and 420 is a long electrode surrounding at least partly or entirely the ablation portion 414. Additionally or alternatively, the electrodes extend along the ablation portion 414, for example along a long axis of the ablation portion 414.

According to some exemplary embodiments, each electrode of electrodes 416, 418 and 420 is formed from at least one electric wire. In some embodiments, the wires are located on or at an outer surface of the ablation portion 414, for example to allow contact between one or more of the electrodes with a tissue. In some embodiments, the electric wires at least partly or entirely surround the ablation portion 414, and/or extend along a long axis of the ablation portion 414. In some embodiments, a length of at least one electrode of electrodes 416, 418 and 420 is at least 0.5 mm, for example at least 1 mm, at least 1.5 mm, at least 2 mm, at least 3 mm, at least 4 mm, at least 5 mm, at least 8 mm, at least 10 mm, at least 12 mm, at least 15 mm, at least 20 mm, or any intermediate, smaller or larger value.

According to some exemplary embodiments, the ablation portion 414 is expandable, and configured to move between a collapsed state, for example when the device 404 passes within a blood vessel, a sheet or a tube, towards an ablation target region, and an expanded state, for example when the ablation portion 414 is expanded to place one or more of the electrodes 416, 418 and 420 in contact with a surface of a tissue in a target ablation region. In some embodiments, the ablation portion 414 is configured to move between a collapsed state and an expanded state and/or between an expanded state and a collapsed state, in response to a signal received from outside the subject body, optionally a signal received from the control unit 406. In some embodiments, the signal is a mechanical signal, an electric signal, a hydraulic signal and/or a pneumatic signal. In some embodiments, the ablation portion 414 is configured to reversibly expand and/or to collapse, optionally when receiving the signal.

According to some exemplary embodiments, the ablation portion 414 comprises a mesh structure, configured to move between a collapsed state and an expanded state. Optionally, the mesh structure comprises an inner lumen. In some embodiments, the mesh structure is formed from electrically insulated wires or electrically insulated stripes. In some embodiments, at least one electrode of electrodes 416, 518 and 420 pass within the mesh structure, and surround at least partly the mesh structure. Optionally, the at least one electrode pass within and is optionally interlaced with the electrically insulated wires or electrically insulated stripes forming the mesh structure.

According to some exemplary embodiments, the ablation portion 414, which optionally comprises the mesh structure, includes a central inflatable portion, for example a balloon. Optionally, the balloon is located in the inner lumen of the mesh structure. In some embodiments, when the device 404 passes within a blood vessel or within a sheet or a tube, towards a target region in a body cavity, the balloon is deflated, for example to maintain the ablation portion 414 in a collapsed state. In some embodiments, when reaching a target region in the body cavity, the balloon is inflated, for example via a signal received from the control unit 406, to expand the ablation portion 414 and to push at least one electrode of electrodes 416, 418 and 420 against the tissue surface. In some embodiments, expansion of the ablation portion 414 within the body cavity, for example by inflating the balloon, is used to anchor the ablation portion 414 or the device 404 with the subject body.

According to some exemplary embodiments, the ablation portion 414 comprises one or more sensors, for example sensor 422. Alternatively, the sensor 422 is coupled to other parts of the body 208. In some embodiments, the sensor 422, is configured to measure at least one of, pressure applied on the tissue by the ablation portion 414 and/or electrical conductivity of the tissue. In some embodiments, measuring the electrical conductivity of the tissue by the sensor 422 allows, for example, to determine results and/or efficacy of a pulse field ablation delivered to the tissue. The delivered PFA is used to reduce or stop electrical conductivity by the ablated tissue. Sensors as discussed herein may also be employed in any of the other embodiments discussed herein which include balloons.

According to some exemplary embodiments, the device 404 further comprises at least one additional electrode, for example a point electrode, a ring electrode or a long electrode, for example electrode 424. In some embodiments, the at least one additional electrode 424 is coupled to the ablation region 414, for example to a distal end of the ablation region 414, or is located distally to the ablation region 414. In some embodiments, the at least one additional electrode 424 is used to deliver an electric field to the tissue, optionally in combination with one or more of the electrodes 416, 418 and 420. Alternatively or additionally, the at least one additional electrode 424 is used to measure electrical properties of the tissue contacting the electrode, for example skin conductivity of the tissue. In some embodiments, measuring the electrical properties of the tissue is optionally performed in combination with the at least one sensor 422. In some embodiments, measuring of the electrical properties of the tissue, for example the ablated tissue, using the at least one additional electrode 424 is used to determine an effect or an efficacy of the PFA delivered to the tissue.

According to some exemplary embodiments, the device 404 is optionally controlled by the control unit 406. In some embodiments, the control unit 406 comprises a control circuitry, for example controller 425 and a pulse generator 426 coupled to the controller 425. In some embodiments, the controller 425 is configured to signal the pulse generator 426 to generate an electric field and to deliver the electric field to one or more of the electrodes of the ablation portion 414, for example electrodes 416, 418 and 420. In some embodiments, the electric field is generated according to indications and/or values of electric field parameters stored in a memory of the control unit, for example memory 428. In some embodiments, the electric field parameters comprise at least one of, intensity, duration of an electric field pulse, number of pulses in a train of pulses, number of trains, and/or interval length between pulses. In some embodiments, the memory 428 also stores history of previous delivery of electric fields and/or preferred parameter values for a specific subject or a group of subjects.

According to some exemplary embodiments, the electric field is delivered via an electrode switch 430 to one or more pairs of electrodes of the ablation portion 414. In some embodiments, the one or more electrode pairs is selected by the controller 425 or by input signals from a user of the system that are received by a user interface. In some embodiments, the one or more electrode pair is selected according to a location of the ablation portion in the blood vessel. Alternatively or additionally, the one or more electrode pair is selected according to a shape and/or size of a target, for example a desired, ablated region, for example according to a length and/or width of the target ablated region. In some embodiments, the controller and/or a user determines an intensity value of the electric field according to a location of the ablation portion 414 and/or a depth of the target ablation portion. In some embodiments, controlling the size and/or shape of ablated region allows, for example, to minimize or prevent damage to untargeted tissue, such as nerves in the wall of the blood vessel.

According to some exemplary embodiments, the user interface 432 is configured to receive input from a user of the system and/or to generate and deliver at least one human detectable indication to the user. In some embodiments, the user interface 432 generates and delivers at least one human detectable indication when the electric field is delivered and/or when the delivery is stopped. In some embodiments, the at least one human detectable indication comprises an audio indication and/or a visual indication.

According to some exemplary embodiments, the control unit 406 comprises a communication circuitry 434 configured to connect the control unit 406 to one or more devices, for example remote devices, and/or units. In some embodiments, the communication circuitry 434 connects the control unit 406 to the one or more devices via wireless signals or via at least one wire. According to some exemplary embodiments, the control unit 406 is used for mapping the electrical properties, for example electrical conductivity of the tissue before, during and/or following the delivery of the electric field. In some embodiments, the mapping is performed in order to get feedback on the efficacy of the PFA treatment. In some embodiments, the control unit 406 optionally comprises an internal mapping circuitry 436 configured to deliver a stimulus to the tissue, optionally via at least one electrode of the device 404, for example electrode 424 and measure the electrical conductivity of the tissue using the same or at least one different electrode of the device 404. Alternatively or additionally, the mapping circuitry 436 measures the electrical conductivity of the tissue using one or more sensors of the device, for example sensor 422. In some embodiments, the mapping circuitry is an external mapping unit, for example unit 438, in communication with the control unit via the communication circuitry 434.

### Exemplary Ablation Adjustments

According to some exemplary embodiments, a user of the ablation device or the ablation system can set the ablation parameters to generate an ablated region having a desired size, shape and/or location. In some embodiments, the ablation parameters are adjusted automatically by the system, for example according to a location, shape and/or size of a planned ablated region. Alternatively, a user of the system adjusts the ablation parameters manually, using a user interface of the system, for example user interface 432 shown in FIG. 4, and optionally based on suggestions presented by the system.

According to some exemplary embodiments, the ablation parameters comprise parameters of the electric field delivered to the tissue. Alternatively or additionally, the ablation parameters comprise at least one of, number of electrodes to be used for delivery of the electric field, the at least one specific set of electrodes to be used for delivery of the electric field, location of the electrodes on the ablation portion of the device, timing of electric field delivery when optionally using two or more pairs of electrodes simultaneously or intermittently and/or electric field parameters per each electrode pair when using two or more electrode pairs.

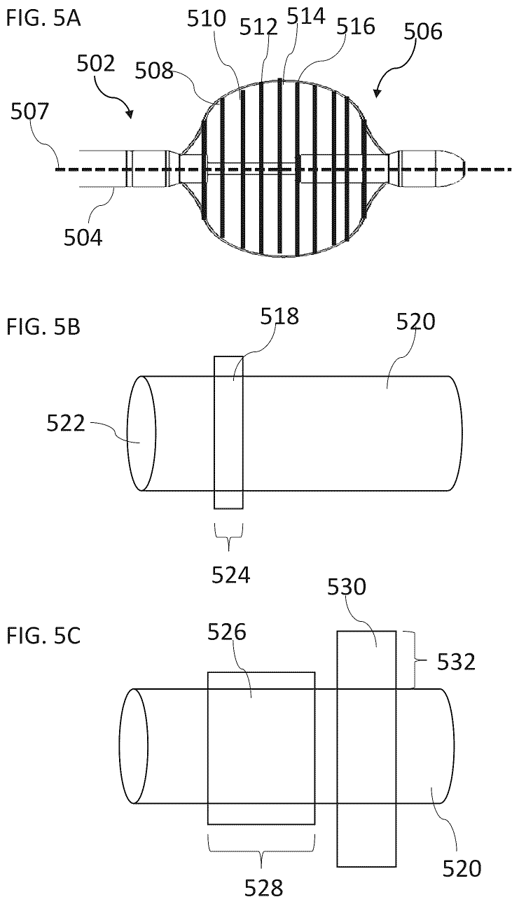

Reference is now made to FIGS. 5A-5C depicting selection of electrode pairs, for example to generate a specific ablated region, according to some exemplary embodiments.

According to some exemplary embodiments, an ablation device 502 comprises a body 504 having an expandable ablation portion 506. In some embodiments, the ablation portion comprises a plurality of electrodes, for example electrodes 508, 510, 512, 514 and 516, surrounding the ablation portion 506, the electrodes optionally configured for use in a PFA procedure. In some embodiments, each of the electrodes has a different axial location along a long axis 507 of the body 504. In some embodiments, each or at least some of the electrodes are separately connected to a control unit, for example to allow selection of electrodes or electrode pairs, for delivery of an electric field to the tissue. In some embodiments, each or at least some of the electrodes are electrically insulated from other electrodes between the ablation portion 506 and the control unit, for example to allow separate electrification of the electrically insulated electrodes from the rest of the electrodes of the ablation portion.

According to some exemplary embodiments, for example as shown in FIG. 5B, a user of the device 502 plans to generate an ablated region 518 in a tissue surrounding a body cavity, for example blood vessel 520. In some embodiments, in order to generate the planned ablated region 518, the ablation portion is placed within a lumen 522 of the blood vessel while the electrodes contact the wall of the blood surface at an axial position of the planned ablated region. Optionally, the ablation portion is expanded in order to push the electrodes against the blood vessel wall, and ensure the contact between the electrodes and the blood vessel wall.

According to some exemplary embodiments, once the ablation portion 506 with the electrodes expands and contacts the blood vessel wall, an electric field is delivered to the tissue via a selected set of electrodes. In some embodiments, the set of electrodes is selected according to an axial length 524 of the planned ablated region 518. In some embodiments, in order to generate the ablated region 518, the electric field is delivered via electrodes 510 and 512 of the ablation portion 506. In some embodiments, for example as shown on FIG. 5C, when the planned ablated region, for example region 526 has a larger length 528, the electric field is delivered between additional adjacent electrode pairs and/or between a pair of distant electrodes, for example electrodes 508 and 516.

According to some exemplary embodiments, a user plans to generate two or more spaced apart ablated regions, for example regions 526 and 530. In some embodiments, the two or more spaced apart ablated regions 526 and 530 are generated by selecting pairs of electrodes that are separated by one or more electrodes or electrode pairs that are not electrified.

According to some exemplary embodiments, an ablated region 530 having depth 532 is generated by adjusting an intensity of the electric field delivered by the at least one electrode pair used for ablation. In some embodiments, when two ablation regions are generated simultaneously, a user or a system adjusts the electric field intensity such that an electric field delivered by a first set of electrodes for generating the ablated region 530 has a higher intensity relative to the intensity of the electric field delivered by a second set of electrodes for generating the ablated region 526.

### Exemplary Ablation Process

According to some exemplary embodiments, the ablation device is used for non-thermal pulse field ablation of tissue, for example heart tissue. In some embodiments, the ablation device is used to perform PFA in a subject diagnosed with an arrhythmia caused by abnormal electrical conductivity through heart tissue. In some embodiments, the PFA ablates heart tissue that is involved in conductance of electricity.

According to some exemplary embodiments, before and/or after the delivery of an electric field to the heart tissue, the heart tissue is paced and the electrical conductivity through the heart tissue is mapped. In some embodiments, the ablation device comprises at least one pacing electrode for pacing the heart tissue. Optionally, the ablation device comprises one or more electrodes for mapping electrical conductivity following the pacing. Alternatively, a mapping catheter is introduced together with the ablation device, and the ablation device is used for ablation and for pacing. Alternatively, the ablation device is used only for ablation, and the mapping catheter or an additional device is used for pacing.

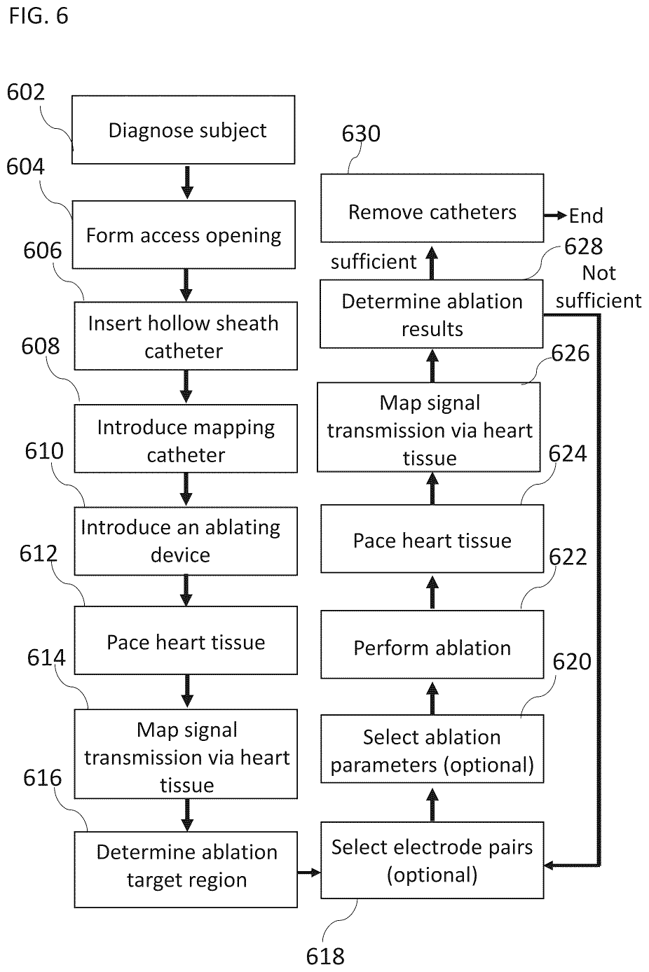

Reference is now made to FIG. 6, depicting an ablation process of heart tissue, according to some exemplary embodiments.

According to some exemplary embodiments, a subject is diagnosed at block 602. In some embodiments, the subject is diagnosed with an arrhythmia, with an irregular heart pace. In some embodiments, the subject is diagnosed with a ventricle arrhythmia or with an atrial arrhythmia, for example with atrial fibrillation.

According to some exemplary embodiments, an access opening is formed in the body of the subject, for example the diagnosed subject, at block 604. In some embodiments, the access opening is formed in the subject femoral vein.

According to some exemplary embodiments, a hollow sheath catheter is inserted through the opening, at block 606. In some embodiments, the distal end of the hollow sheath catheter is advanced, optionally via the vein, into the left atrium of the heart.

According to some exemplary embodiments, a mapping catheter is introduced into the heart, at block 608. In some embodiments, the mapping catheter is introduced via the sheath into the heart, for example into the left atrium.

According to some exemplary embodiments, an ablation device, for example ablation device 404 shown in FIG. 4, is introduced into the heart, for example into the left atrium, at block 610. In some embodiments, the ablation device is introduced into the left atrium via the hollow sheath. In some embodiments, the ablation device 404 is an ablation catheter.

According to some exemplary embodiments, the heart is paced, at block 612. In some embodiments, the heart is paced using one or more electrodes of the ablation device, for example electrode 424 shown in FIG. 4. Alternatively, a separate pacing catheter is inserted into the heart at block 610 in addition to the ablation device, and is used for the pacing at block 612. In some embodiments, the ablation device or the separate pacing catheter stimulate the heart to create specific heart beat signals that can be received by the mapping catheter.

According to some exemplary embodiments, signals transmission through the heart tissue is mapped, at block 614. In some embodiments, the mapping catheter is used to map the signals transmission, in response to the pacing delivered at block 612. In some embodiments, the mapping is used to determine an anatomy of the heart, for example the anatomy of the left atrium. In some embodiments, the mapping is used to identify abnormal signal transmission paths in the heart tissue, for example in the left atrium.

According to some exemplary embodiments, an ablation target region is determined, at block 616. In some embodiments, the ablation target region is determined based on the mapping performed at block 614. In some embodiments, the ablation target region is determined based on the identified signal transmission paths and/or based on anatomical and physiological properties of the heart, for example the left atrium. In some embodiments, the ablation target region is a region along the identified abnormal signal transmission path that does not include or is remote from tissue that should not be ablated, for example nerve tissue. Optionally, determining an ablation target comprises determining or planning a shape and/or size of an ablated region to be formed in the ablation target region.

According to some exemplary embodiments, electrode pairs are optionally selected at block 618. In some embodiments, the electrode pairs are selected according to a shape and/or size of the ablation region determined at block 616. In some embodiments, one or more electrode pairs are selected, for example according to a target length, optionally an axial target length of the determined ablation region.

According to some exemplary embodiments, at least one ablation parameter is optionally selected at block 620. In some embodiments, values of the at least one ablation parameters are selected. In some embodiments, the ablation parameter comprises at least one of, intensity of an electric field, frequency of the electric field, duration of a pulse of the electric field, number of pulses of the electric field to be delivered to the tissue in a train of pulses or overall in a PFA treatment, number of trains of pulses needed in order to achieve the shape and/or size of the determined ablation region, and/or location of the ablation device within the body cavity.

According to some exemplary embodiments, ablation is performed at block 622. In some embodiments, during ablation an electric field is delivered to the tissue via one or more pairs of electrodes of the ablation device, for example electrode pairs of the ablation portion of the device. In some embodiments, the electric field is delivered via the electrode pairs optionally selected at block 618, and/or with the ablation parameters and values thereof optionally selected at block 620. In some embodiments, the electric field is delivered to the tissue with parameter values that generate electroporation and optionally irreversible electroporation of the tissue at the ablation region. In some embodiments, the ablation is a non-thermal ablation, optionally causing a change in temperature of the tissue at the target ablation region which is smaller than 10 degrees Celsius, for example a change which is smaller than 5 degrees Celsius, a change which is smaller than 2 degrees Celsius, or any intermediate, smaller or larger value. Alternatively, the ablation does not change the temperature of the tissue at the target ablation region.

According to some exemplary embodiments, the ablation is performed by placing at least one electrode pair of the ablation device in contact with a surface of heart tissue and delivery of the electric field through the tissue contacting electrode pair. In some embodiments, the at least one electrode pair is placed in contact with the tissue by expanding an ablation portion of the ablation device such that the expanded ablation portion pushes the electrode pair against the tissue.

According to some exemplary embodiments, the electric field is delivered at block 622 with an intensity value, for example voltage, in a range between about 300 volts and about 3000 volts, for example between about 300 volts and about 1000 volts, between about 500 volts and about 1500 volts, between about 1000 volts and about 3000 volts, or any intermediate, smaller or larger range of values. In some embodiments, the electric field is delivered with a frequency value in a range between about 15 and about 1000 kHz, for example in a range between about 15 and about 250 kHz, in a range between about 50 and about 500 kHz, in a range between about 100 and about 1000 kHz, or any intermediate, smaller or larger range of values. In some embodiments, the electric field is delivered during a time period in a range between about 0.05 and about 500 milliseconds (ms), for example during a time period in a range between about 0 and about 100 ms, during a time period between about 50 and about 200 ms, during a time period between about 100 and about 500 ms, or any intermediate, smaller or larger range of values.

According to some exemplary embodiments, the heart tissue is paced at block 624. In some embodiments, the heart tissue is paced following the ablation, by at least one electrode of the ablation device. Alternatively, the heart tissue is paced by a pacing catheter for example by at least one electrode of the pacing catheter.

According to some exemplary embodiments, signals transmission through the heart tissue is mapped, at block 626. In some embodiments, the signals transmission is mapped following the ablation. In some embodiments, the mapping is performed using a mapping catheter. Alternatively, the mapping is performed by one or more electrodes of the ablation device.

According to some exemplary embodiments, results of the ablation procedure are determined at block 628. In some embodiments, the ablation results are determined based on the mapping performed at block 626. In some embodiments, the results of the ablation procedure are determined by the system 402. In some embodiments, the results of the ablation are determined by comparing the signals transmission mapped at block 614 and the signals transmission mapped at block 626, or by determining a relation therebetween. In some embodiments, the results of the ablation determined at block 628 indicate whether or not the ablation procedure succeeded to reach a target physiological goal, for example ablation of an unwanted signals transmission path in the heart and/or ablation of an unwanted pacing source in the heart tissue.

According to some exemplary embodiments, if the ablation is sufficient to reach the target physiological goal, the devices and catheter introduced into the body are removed, at block 630. Alternatively, if the ablation is not sufficient to reach the target physiological goal, blocks 618, 620, 622, 624, 626 and 628 are repeated. Optionally, the ablation device is repositioned inside the heart prior to repeating the blocks.

Exemplary Ablation Device with Twisted Electrode Wires

According to some exemplary embodiments, an ablation catheter, for example an ablation device comprises an elongated body with an ablation portion which is configured to move between a collapsed state and an expanded ring-shaped, lasso-like structure. In some embodiments, the ablation portion comprises two or more electrodes twisted around an expandable scaffold forming the ablation portion that is configured to form the ring-shaped structure when expanded. In some embodiments, the electrodes for example wire electrodes are twisted around the scaffold.

Ablation Device Including an Ablation Portion having Axially Extending Electrode Wires

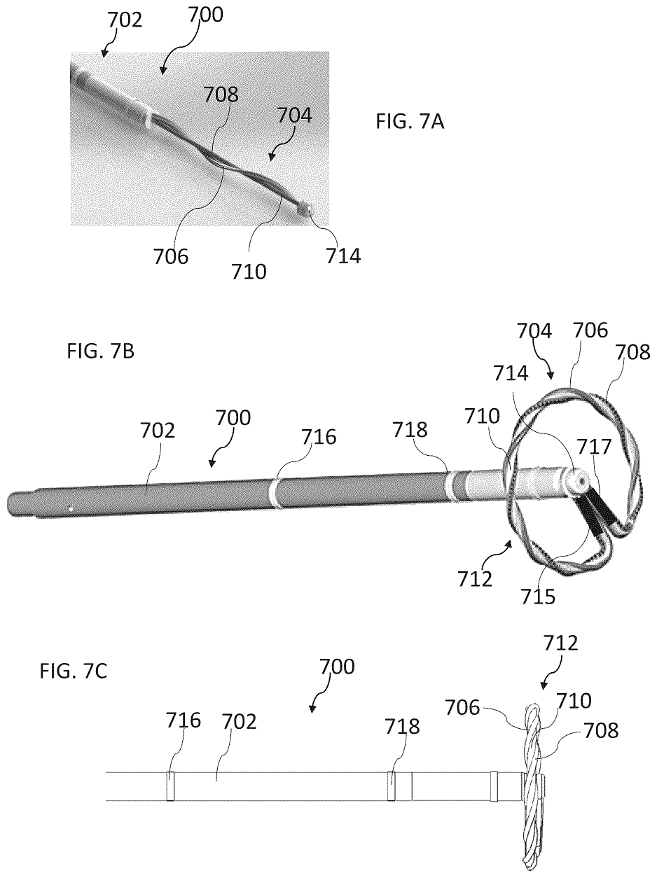

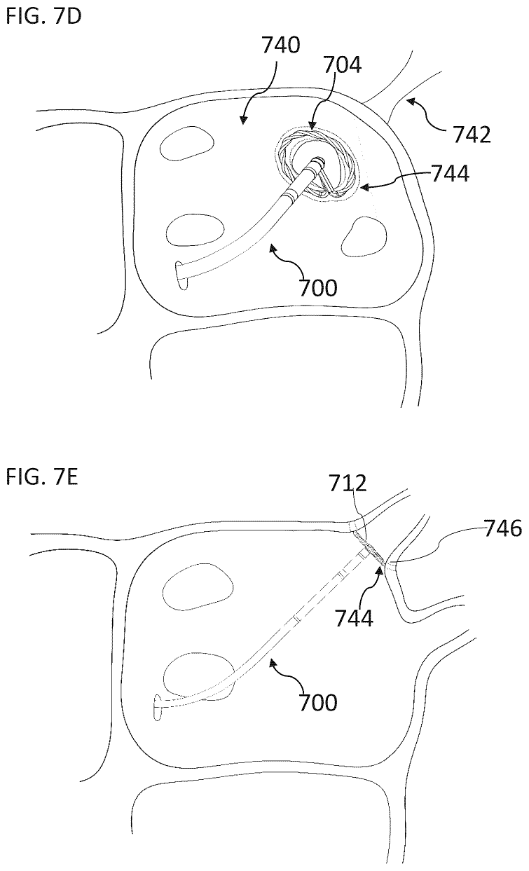

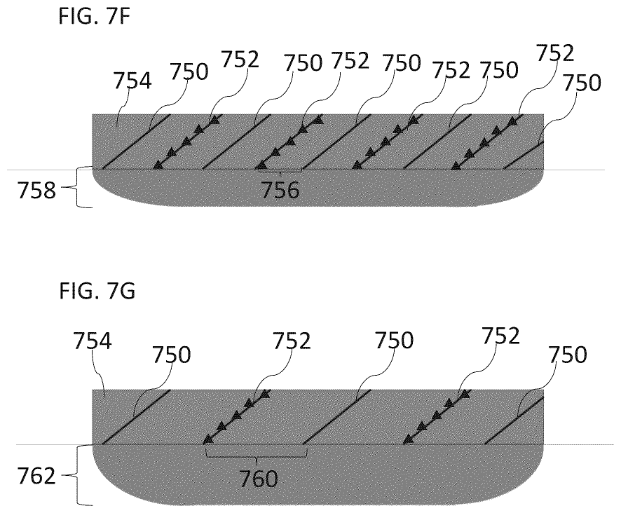

Reference is now made to FIGS. 7A-7C, wherein there is depicted an ablation device having an ablation portion configured to expand into a ring-shaped structure, according to some exemplary embodiments.

According to some exemplary embodiments, an ablation device 700, for example an ablation catheter, comprises an elongated body 702 and an expandable ablation portion 704. In some embodiments, the expandable ablation portion 704 is positioned at a distal end of the body 702. In some embodiments, the expandable ablation portion 704 is configured to move between a collapsed state, for example as shown in FIG. 7A, and an expanded state, for example as shown in FIG. 7B.

According to some exemplary embodiments, the ablation portion 704 is formed from an expandable and optionally reversibly expandable scaffold. In some embodiments, the scaffold is formed from a shape memory alloy material, for example Nitinol, optionally configured to expand into an expanded state when the scaffold is in a relaxed state. In some embodiments, the scaffold, optionally formed from one or more wires is configured to form a ring-shaped structure, when the scaffold is expanded.

According to some exemplary embodiments, the device 700 comprises at least two electrodes, for example wire electrodes 706 and 708 twisted around the scaffold forming the ablation portion 704, the electrodes optionally configured for use in a PFA procedure. In some embodiments, the wire electrodes are twisted around a support structure, for example the at least one wire forming the scaffold, for example wire 710. In some embodiments, the scaffold, for example the wire is an electrically insulated wire or is formed from a non-conductive material. In some embodiments, the electrodes wires are also twisted around each other each, for example as shown in FIG. 7B. In some embodiments, the wire electrodes 706 and 708 extend from the ablation portion towards a proximal end of the body 702 optionally configured to be located outside the patient body. In some embodiments, the support structure comprises one or more spline elements, for example wire 710. In some embodiments, two or more electrodes, for example electrode wires 706 and 708 are wrapped around the spline elements, optionally, at a specific distance therebetween, along at least 50%, for example at least 60%, at least 70%, at least 80%, at least 90% or any intermediate, smaller or larger length of the spline element.

According to some exemplary embodiments, in an expanded state, for example as shown in FIGS. 7B and 7C, the expandable scaffold of the ablation portion 704 forms a circular ring shape, with the wire electrodes 706 and 708 located on the circumference of the ring. In some embodiments, the body 702 is mechanically coupled to the center of the ring 712.

According to some exemplary embodiments, the body 702 comprises a flexible shaft configured to bend within a sheet when navigated from outside the subject body into a body cavity, for example a blood vessel or into a heart of the subject. In some embodiments, the body 702 comprises one or more distal electrodes 714 located at a distal tip of the body 702, and optionally distally to the expandable ablation portion 704. Additionally or alternatively, the body 702 comprises at least one body electrode optionally a ring electrode, for example electrodes 716 and 718 surrounding the body 702. In some embodiments, the at least one distal electrode 714 and/or the at least one body electrode is used for pacing heart tissue and/or for mapping electrical transmission through the heart tissue, and optionally using one or more of the wire electrodes 706 and 708. Alternatively or additionally, the at least one distal electrode 714 and/or the at least one body electrode is used together with the wire electrodes 706 and 708 for delivery of an electric field to the tissue for ablating the tissue.

According to some exemplary embodiments, when delivering an electric field via the wire electrodes 706 and 708, ablation occurs in a tissue region contacting the ablation portion between the wire electrodes. In some embodiments, the wires forming the electrodes are formed from any material with electrical properties that allow the passage of high Voltage of up to 3000 volts. In some embodiments, a width, for example diameter, of a wire electrode is within a range between about 0.05 mm and about 1 mm, for in a range between about 0.05 mm and about 0.2 mm, in a range between about 0.1 mm and about 0.5 mm, in a range between about 0.4 mm and about 1 mm, or any intermediate, smaller or larger range of values. In some embodiments, a distance between the two wire electrodes, for example electrodes 706 and 708 is up to about 4 mm, for example up to about 3 mm, up to about 2 mm, up to about 1 mm, up to about 0.5 mm, or any intermediate, smaller or larger value. In some embodiments, a cross section of the wire electrodes is circular, elliptical, oval, or a polygon.

Optionally, the ablation portion is at least partly coated, for example to prevent damage to tissue contacting the ablation portion. In some embodiments, the coating is a dielectric coating, or a dielectric cover.

According to some exemplary embodiments, at least some portions of the wire electrodes are electrically insulated, for example intersection regions and/or regions of the wire electrodes located proximally to the ablation portion 704. In some embodiments, connecting regions between the wire electrodes and/or the wire scaffold 710, and the body 702 are covered with an electric insulating material, for example an insulating sheet or sleeve on each electrode, for example insulating sleeves 715 and 717.

According to some exemplary embodiments, for example as shown in FIG. 7D, and 7E, during a PFA procedure, optionally to treat atrial fibrillation the device 700 is introduced into the left atrium (LA) 740, and the ablation portion 704 is expanded inside the pulmonary vein 742. In some embodiments, the ablation portion 704 is expanded within an opening 744 of the pulmonary vein 742 in the LA 740, for example at a distance of up to 7 mm, up to 5 mm, up to 3 mm, up to 2 mm, up to 1 mm, or any intermediate, smaller or larger distance from the LA into the pulmonary vein.

According to some exemplary embodiments, for example as shown in FIG. 7E, expansion of the ablation portion inside the pulmonary vein 742 pushes the ring structure 712 of the expanded ablation portion against a wall 746 of the pulmonary vein. In some embodiments, expansion of the ablation portion anchors the ablation portion within the blood vessel, for example within the pulmonary vein, and places in contact the wire electrodes 706 and 708 twisted around the ring structure, with the pulmonary vein wall. In some embodiments, delivery of an electric field via the wire electrodes 706 and 708 generates a circular or an arc-shaped ablated region in the wall of the pulmonary vein surrounding at least partly the lumen of the pulmonary vein.

### Exemplary Relationship of Distance Between Electrodes and Ablation Depth

Reference is now made to FIGS. 7F and 7G depicting a relation between a distance between adjacent electrodes and an ablation depth, according to some exemplary embodiments.

According to some exemplary embodiments, one or more electrode wires, for example one or more electrode wire pairs are twisted around an electrode support structure. In some embodiments, an electric field is delivered to a tissue between two adjacent electrode wires. In some embodiments, a distance between the electrode pairs that deliver the electric field to the tissue determines a depth of an ablated region formed by the electric field.

According to some exemplary embodiments, two elongated electrodes, for example electrode wires 750 and 752 are twisted around, and along a length of a support structure 754, for example to form an ablation portion of an ablation device. In some embodiments, a distance between the twisted electrodes, for example distance 756 is maintained fixed along the length of the support structure. Alternatively, the distance 756 varies, forming regions which include a higher number of electrode wires windings compared to other regions of the ablation portion. In some embodiments, an ablation depth 758, for example a depth of an ablation region to which the electric field is delivered is determined according to the distance 756 between the electrodes that deliver the electric field.

According to some exemplary embodiments, for example as shown in FIG. 7G, increasing a distance 760 between adjacent electrodes 750 and 752, increases the ablation depth 762, compared to ablation depth 758 when using distance 756, and similar parameters of the electric field. In some embodiments, a user adjusts the distance between the electrodes prior to a procedure. Alternatively, an ablation device having an ablation portion with a specific distance between electrodes is selected prior to a procedure, for example according to a desired ablation depth at an ablation target and/or at a target location. Optionally, in case an ablation portion comprises regions with different density of electrode windings, a user positions an ablation portion region with a selected winding density in contact with tissue surface at a target location, according to a desired ablation depth in a tissue at the target location.

Exemplary Ablation Devices with an Expandable Mesh Structures

According to some exemplary embodiments, an ablation device comprises an expandable ablation portion configured to expand, for example reversibly expand, into a spherical shape. Optionally, the spherical shape is elongated, extending along a length which is larger than a diameter of a cross-section of the sphere. In some embodiments, the ablation portion comprises two or more long electrodes surrounding the ablation portion, and optionally extending along at least part of the length of the ablation portion. In some embodiments, the expandable ablation portion is formed from an expandable mesh structure comprises the two or more long electrodes.

A potential advantage of using an expandable mesh structure which expands into a spherical shape is that this may allow formation of elongated ablation regions in a wall of a body cavity, which are optionally longer than a ring-shaped ablation portion formed by the device 700.

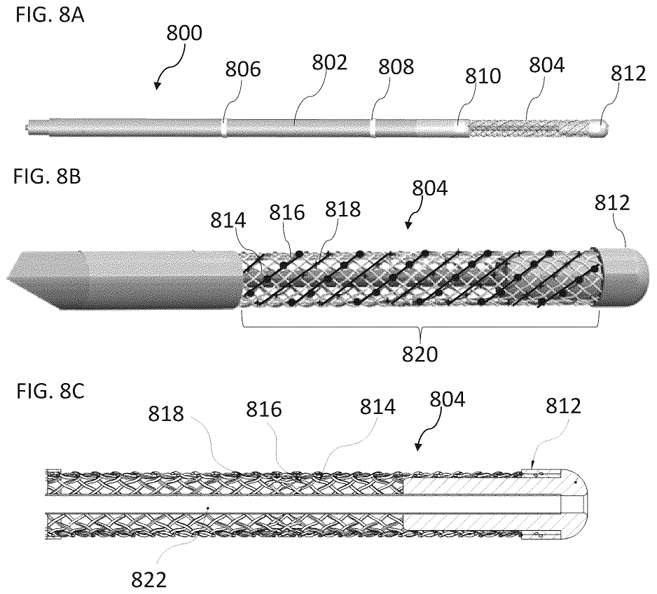

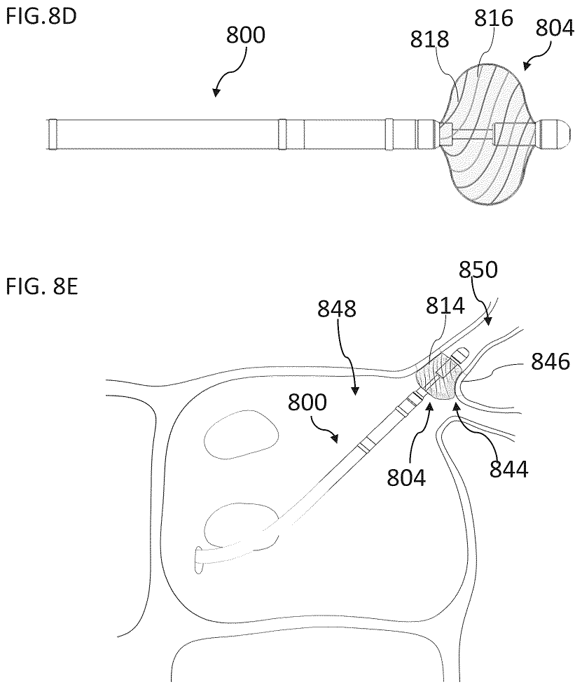

FIGS. 8A-8D relate to an aspect of some embodiments, wherein there is depicted an ablation device with an expandable ablation portion having long electrodes surrounding the ablation portion and extending along a length of the ablation portion, the electrodes optionally configured for use in a PFA procedure, according to some exemplary embodiments.

According to some exemplary embodiments, an ablation device 800 comprises an elongated body 802 which is optionally flexible, for example to allow passage of the device 800 within blood vessels of the body, and an ablation portion 804 at a distal portion of the body 802. In some embodiments, the body 802 comprises one or more electrodes, for example ring electrodes at different locations along the body 802, for example electrodes 806, 808, 810. In some embodiments, the device 800 comprises at least one electrode, for example a ring electrode 812, at a distal location relative to the expandable ablation portion 804 or at a distal end of the ablation portion 804.

According to some exemplary embodiments, for example as shown in FIGS. 8B and 8C, the expandable ablation portion 804 comprises a non-conductive mesh structure 814 having two or more conductive electrode wires, for example electrodes 816 and 818, twisted around a circumference of the mesh structure or interwoven with the mesh structure, and extending along a length 820 of the mesh structure. In some embodiments, the electrode wires are interwoven within the mesh structure 814, while being twisted in a circumference of the mesh structure. Optionally, the mesh structure is formed from a non-conductive polymer. In some embodiments, when the mesh structure expands into a closed spherical structure, for example as shown in FIG. 8D, or into a basket shape having a front opening, the two or more twisted electrode wires surrounding the mesh structure are pushed against a tissue surface of a body cavity. In some embodiments, the two or more electrode wires 816 and 818 are interwoven within the mesh structure 814. In some embodiments, the two or more electrode wires 816 and 818 pass in between wires or stripes forming the mesh structure 814.

According to some exemplary embodiments, the electrodes wires 816 and 818 are substantially parallel to each other with a deviation in a distance between adjacent electrodes of up to 10% along the electrodes length in the ablation portion, when the ablation portion 804 is in a collapsed state and when the ablation portion 804 is in an expanded state. In some embodiments, a distance between adjacent electrodes of the ablation portion, for example electrodes 816 and 818 is up to 5 mm, for example up to 4 mm, up to 3 mm, up to 2 mm, up to 1 mm, up to 0.5 mm or any intermediate, smaller or larger distance between the electrodes. In some embodiments, the electrodes are spaced apart from each other by an electrically insulating region of the ablation portion, for example of the mesh structure.

According to some exemplary embodiments, the mesh structure 814 comprises a central shaft 822, for example a hollow central shaft. In some embodiments, the central shaft 822 is coupled to the body 802 of the device 800. In some embodiments, the central shaft 822 is configured to be pushed, pulled or rolled, for example turned, in order to move the ablation portion between a collapsed state, for example as shown in FIGS. 8A-8C, and an expanded state, for example as shown in FIG. 8D. Alternatively, the ablation portion 804 comprises at least one balloon instead of the central shaft, coupled to the central shaft or within the central shaft. In some embodiments, inflation of the at least one balloon expands the mesh structure 814 into an expanded state, and optionally deflation of the balloon collapses the mesh structure to a collapsed state, for example a collapsed state shown in FIGS. 8A-8C.

According to some embodiments, a diameter of an expanded ablation portion 804 may be in a range of from about 20-35 mm such as, for example from about 20-25 mm, from about 25-30 mm, or from about 30-35 mm, which may be suitable for partial insertion into a pulmonary vein funnel, with about 10 mm or more of the balloon remaining within the left atrium, outside the pulmonary vein.

According to some exemplary embodiments, the at least one electrode 812 or any other electrode optionally located distally to the mesh structure 814 is used for pacing, for example pacing of heart tissue. In some embodiments, one or more of the electrodes, for example ring electrodes 806, 808 and 810 is used for at least one of, delivery of the PFA, pacing and/or mapping of signals, for example electric signals, through heart tissue.

According to some exemplary embodiments, for example as shown in FIG. 8E, the device 800 is used for delivery of PFA to heart tissue. In some embodiments, the ablation portion 804, comprising the mesh structure 814, is expanded within the pulmonary vein opening 844, pushing the electrodes 816 and 818 against the wall 846 of the pulmonary vein. In some embodiments, at least part of the expanded mesh structure 814 is located in the heart, for example in the left atrium 848. Alternatively, the entire mesh structure 814 is located within the pulmonary vein. In some embodiments, an electric field is delivered through the electrodes of the ablation portion to tissue contacting the electrodes, when the ablation portion is expanded within the body cavity and is in contact at least partly with a wall of the body cavity, for example with the wall 846 of the pulmonary vein 850.

According to some exemplary embodiments, by pulling, pushing or rotating the center shaft 822, the mesh structure which is a braided mesh structure, can expend to a balloon-like shape that is very flexible, without sharp edges, that can be positioned at the entrance of the left atrium pulmonary veins (PV) or inside the PV. In some embodiments, the expanded mesh structure is flexible enough to adapt to anatomy of the veins, whether the veins have complex anatomy, stenosis, unsmooth surfaces or a smaller than usual opening. In some embodiments, the mesh structure is small enough to enter any PV and expand to an exact size and create contact between the electrodes and the PV circumference, at the contact region between the expanded mesh structure and the PV wall.

Optionally, at least one electrode of the mesh structure 814 may each have its own wire so that the electrodes may be controlled separately. For example, there may be six electrodes interwoven in the braid of the mesh structure 814, each electrode having its own wire, so that any pair of electrodes from the six electrodes may be selected to provide ablation, depending on the area to be ablated, according to some embodiments. Similarly, any electrode or electrode portion of any device discussed herein such as, for example, shown in any of FIGS. 5A, 8A-10E, 13A-20C may be provided its own wide so that individual electrodes or electrode portions may be controlled separately.

Further optionally, preselected portions of any of electrodes or electrode portions of the structure of any device discussed herein such as, for example, shown in any of FIGS. 5A, 8A-10E, 13A-20C may be provided with an electrically isolating material such as, for example, an enamel, ePTFE (polytetrafluoroethylene), polyimide, FEP (fluorinated ethylene propylene), PFA (perfluoroalkoxy alkanes), or any other insulative/isolating material discussed herein, or any other suitable material that may insulate one or more portions of the electrodes. This may allow ablation of a preselected portion of tissue, depending on the portion(s) of the electrodes not provided with isolating material, according to some embodiments.

A potential advantage of maintaining the electrodes substantially parallel to each other may be to ensure that the electric field depth and the ablation region remains consistent or substantially consistent with deviation in an average distance between adjacent electrodes in an expanded state of up to 20% of the distance between adjacent electrodes, along the electrode lengths in the ablation portion when in the collapsed state and when the ablation portion is in an expanded state. Optionally, the average deviation distance between adjacent electrodes in an expanded state may be from 30-40%, up to 8%, up to 5%, or up to 3%, up to 5%, up to 3%, up to 1% or any intermediate, smaller or larger deviation percentage in average deviation distance between adjacent electrodes in the ablation depth along the ablation line, according to some embodiments.

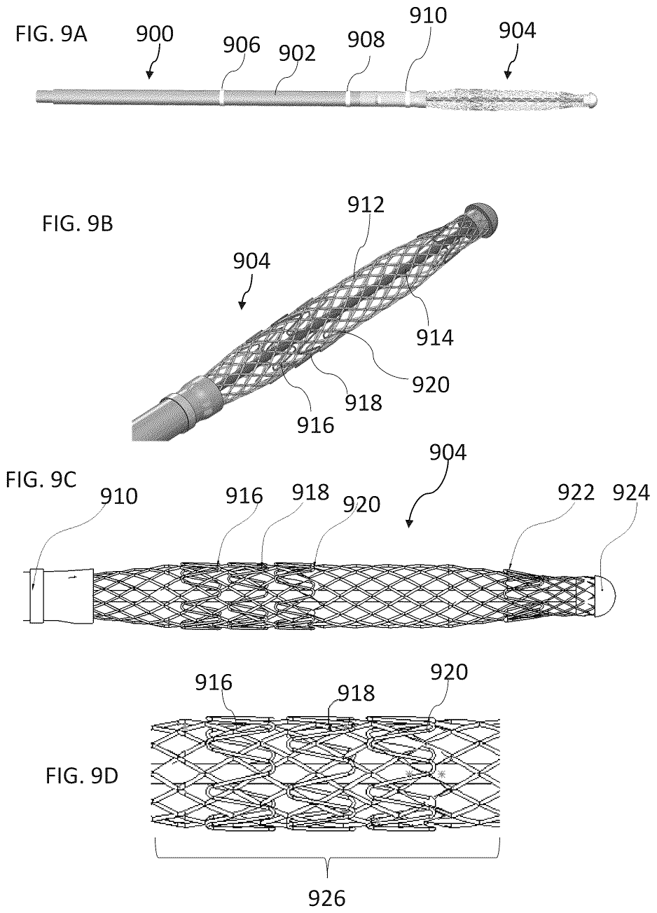

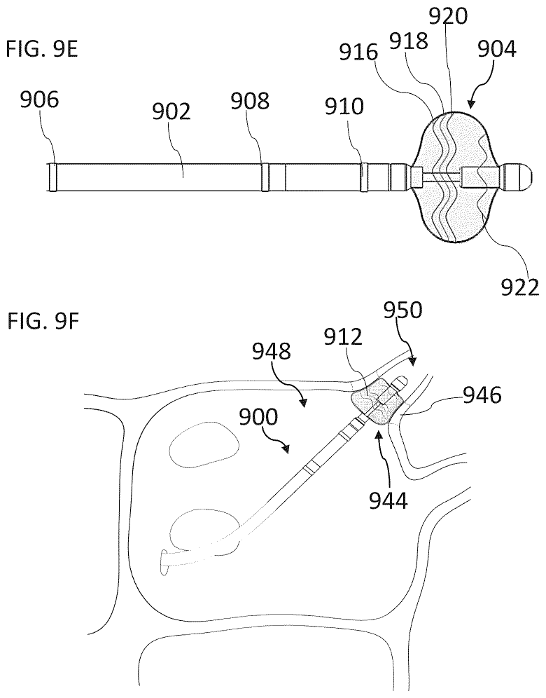

Reference is now made to FIGS. 9A-9E, depicting an ablation device having an expandable ablation portion with electrodes in a zigzag or helical pattern, according to some exemplary embodiments.

According to some exemplary embodiments, an ablation device 900 comprises an elongated body 902 which is similar to body 802, and an ablation portion 904 which is part of the body 902 or is coupled to the body 902. In some embodiments, the body is hollow and/or is flexible, optionally comprising a hollow and/or a flexible shaft. In some embodiments, the ablation portion 904 is an expandable ablation portion, configured to move between a collapsed state, for example when the ablation device travels within a tube or a sheet catheter into a body cavity, and an expanded state, when the ablation portion expands and contacts a wall of the body cavity, for example a wall of a blood vessel.

According to some exemplary embodiments, the device 900 comprises one or more electrodes which are optionally configured for use in a PFA procedure, for example ring electrodes located proximally to the ablation portion 904, for example closer to an end of the device 900 configured to be positioned outside the body or outside the body cavity. In some embodiments, the proximal electrodes comprise electrodes 906, 908 and 910 which are spaced apart from each other and positioned at different axial positions along the length of the body 902. In some embodiments, electrodes 906, 908 and 910 are arc electrodes, surrounding at least partly the body 902.

According to some exemplary embodiments, for example as shown in FIGS. 9B and 9C, the ablation portion comprises a mesh structure 912, for example a braided mesh structure, surrounding a central shaft 914, which is optionally hollow, for example a shaft similar to shaft 822. In some embodiments, the central shaft 914 is configured to be pushed, pulled or rolled, for example turned, in order to move the ablation portion between a collapsed state, for example as shown in FIGS. 9A-9D, and an expanded state, for example as shown in FIG. 9E. Alternatively, the ablation portion 904 comprises at least one balloon instead of the central shaft, coupled to the central shaft or within the central shaft. In some embodiments, inflation of the at least one balloon expands the mesh structure 912 into the expanded state shown in FIG. 9E, and optionally deflation of the balloon collapses the mesh structure 912 to a collapsed state, for example a collapsed state shown in FIGS. 9A-9D.

According to some embodiments, a diameter of an expanded ablation portion 904 may be in a range of from about 20-35 mm such as, for example, from about 20-25 mm, from about 25-30 mm, or from about 30-35 mm, which may be suitable for partial insertion into a pulmonary vein funnel, with about 10 mm or more of the balloon remaining within the left atrium, outside the pulmonary vein.

According to some exemplary embodiments, for example as shown in FIGS. 9B and 9C, the mesh structure 912 is a non-conductive mesh structure having two or more conductive electrodes, for example electrode wires, surrounding the mesh structure, for example electrodes 916, 918 and 920. In some embodiments, each of the electrodes 916, 918 and 920 is located at a different axial position along a length 926 of the body 902, for example as shown in FIG. 9D. In some embodiments, each of the electrodes or at least one of the electrodes 916, 928 and/or 920, has a zigzag or a helical shape surrounding at least partly the ablation portion 904. Alternatively at least one of the electrodes has a straight linear shape surrounding the ablation portion. In some embodiments, the electrodes are spaced apart from each other by an electrically insulating region of the ablation portion, for example of the mesh structure.

According to some exemplary embodiments, the mesh structure is formed from a non-conductive polymer. In some embodiments, when the mesh structure expands into a closed spherical structure, for example as shown in FIG. 9E, or into a basket shape having a front opening, the two or more twisted electrode wires surrounding the mesh structure are pushed against a tissue surface of a body cavity. In some embodiments, the two or more electrodes 916, 918 and/or 920 are interwoven within the mesh structure 912. In some embodiments, the two or more electrodes 916, 918 and/or 920 pass in between wires or stripes forming the mesh structure 912.

According to some exemplary embodiments, at least some of the electrodes, for example electrodes 916, 918 and/or 920 are substantially parallel to each other with a deviation in a distance between adjacent electrodes of up to 10% along the electrodes length in the ablation portion. In some embodiments, a distance between adjacent electrodes of the ablation portion, for example electrodes 916, 918 and 920 is up to 5 mm, for example up to 4 mm, up to 3 mm, up to 2 mm, up to 1 mm, up to 0.5 mm or any intermediate, smaller or larger distance between the electrodes.

According to some exemplary embodiments, the ablation portion comprises at least one distal electrode, for example electrode 922, close to a distal end or a distal tip 924 of the ablation portion. In some embodiments, the electrode 922 is similar to electrodes 916, 918 and 920. In some embodiments, the electrode has a zigzag or a helical shape, surrounding at least partly the ablation portion 904. Alternatively, the electrode 922 has a straight linear shape surrounding the ablation portion 904. In some embodiments, the at least one electrode 922 is used for pacing of the tissue, for example by delivery of an electric field that is sufficient to pace the tissue. Alternatively, or additionally the at least one electrode is used for mapping signal transmission through the tissue. In some embodiments, at least one of the electrodes 906, 908 and 910 is used for pacing of the tissue and/or for mapping signal transmission, optionally in combination with one or more of the electrodes 916, 918, 920 and/or 922. Optionally, the distal tip 924 of the ablation portion 904, for example of the mesh structure 912 forming the ablation portion 904 is used for at least one of delivery of PFA, pacing a tissue and/or mapping signal transmission through the tissue.

According to some exemplary embodiments, for example as shown in FIG. 9F, the device 900 is used for delivery of PFA to heart tissue. In some embodiments, the ablation portion 904, comprising the mesh structure 912, is expanded within the pulmonary vein opening 944, pushing the electrodes 916, 918 and 920 against the wall 946 of the pulmonary vein. In some embodiments, at least part of the expanded mesh structure 912 is located in the heart, for example in the left atrium 948. Alternatively, the entire mesh structure 912 is located within the pulmonary vein 950. In some embodiments, an electric field is delivered through the electrodes of the ablation portion to tissue contacting the electrodes, for example electrodes 916, 928 and 920. In some embodiments, the electric field is delivered when the ablation portion is expanded within the body cavity and is in contact at least partly with a wall of the body cavity, for example with the wall 946 of the pulmonary vein 950.

Exemplary Ablation Device with an Inflatable Scaffold Structure

According to some exemplary embodiments, an ablation device comprises an elongated body having an expandable portion which includes an arrangement of electrodes. In some embodiments, the expandable portion is an inflatable portion, configured to move between a deflated state, for example when traveling within a sheath, a catheter or a working channel, of a delivery device into a body lumen, and an inflated expanded state, used to anchor or place the inflatable portion or electrodes thereof in contact with walls of the body lumen.

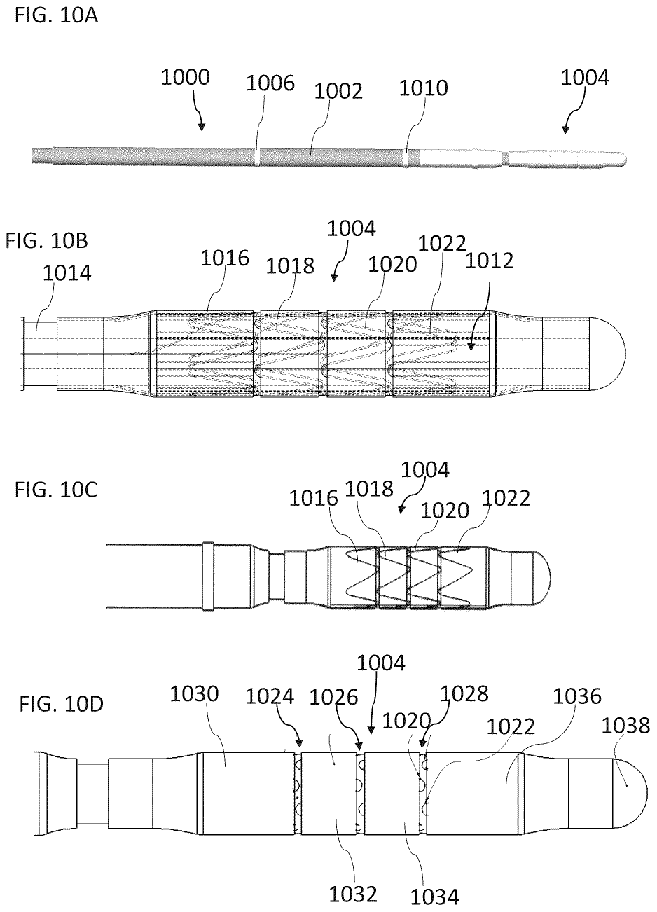

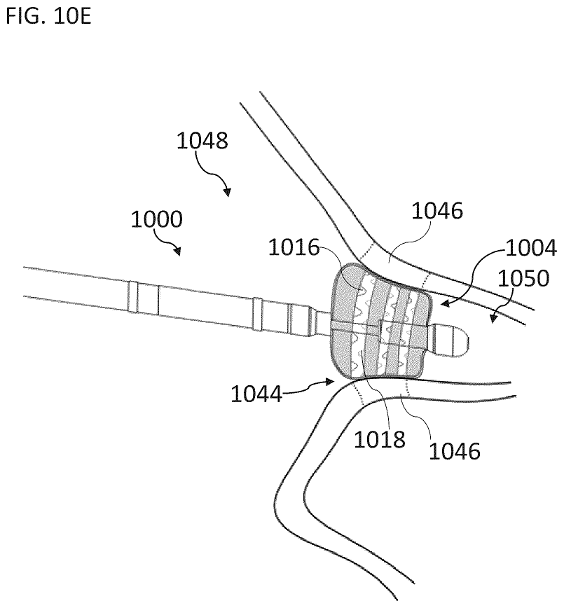

Reference is now made to FIGS. 10A-10D depicting a structure of an ablation device having an inflatable electrode carrying portion, according to some exemplary embodiments.

According to some exemplary embodiments, an ablation device 1000 comprises an elongated body 1002 which is optionally similar to body 902, and an ablation portion 1004 which is part of the body 1002 or is coupled to the body 1002. In some embodiments, the body 1002 is hollow and/or is flexible, optionally comprising a hollow and/or a flexible shaft. In some embodiments, the ablation portion 1004 is an expandable inflatable ablation portion, configured to move between a deflated state, for example when the ablation device travels within a tube or a sheet catheter into a body cavity, and an expanded inflated state, when the ablation portion is inflated and contacts a wall of the body cavity, for example a wall of a blood vessel.

According to some exemplary embodiments, the device 1000 comprises one or more electrodes optionally configured for use in a PFA procedure, for example ring electrodes located proximally to the ablation portion 1004, for example closer to an end of the device 1000 configured to be positioned outside the body or outside the body cavity. In some embodiments, the proximal electrodes comprise electrodes 1006, 1008 and 1010 which are spaced apart from each other and positioned at different axial positions along the length of the body 1002. In some embodiments, electrodes 1006 and 1010 are arc electrodes, surrounding at least partly the body 1002. Alternatively, the electrodes 1006, 1008 and 1010 are circumferential electrodes, surrounding the body 1002.

According to some exemplary embodiments, for example as shown in FIG. 10B, the ablation portion 1004 comprises an inflatable portion 1012, for example a balloon, surrounding a central shaft 1014, which is optionally hollow, for example a shaft similar to shaft 822 or shaft 914. In some embodiments, the central shaft 1014 comprises one or more tubes configured to deliver and remove fluid, for example gas, air or liquid, to and from the inflatable portion 1012. In some embodiments, delivery of fluids into the inflatable portion 1012 inflates the inflatable portion 1012 to an inflated state, optionally pushing the inflatable portion 1012 against a wall of the body lumen, for example body cavity. In some embodiments, removal of fluids from the inflatable portion 1012 deflates the inflatable portion 1012 to a deflated state. In some embodiments, the inflatable portion 1012, for example a balloon, is made from a polymeric material.

According to some exemplary embodiments, the inflatable portion comprises two or more electrodes, surrounding at least partly or completely the inflatable portion 1012. In some embodiments, the electrodes, for example electrodes 1016, 1018, 1020, and 1022 have a zigzag formation, for example zigzag shaped electrodes. Alternatively, the electrodes are straight linear electrodes.

According to some exemplary embodiments, for example as shown in FIG. 10C, the electrodes are mounted on top of, or are attached to, an outer surface of the inflatable portion 1012 or a coating thereof. In some embodiments, the electrodes are arranged a fixed distance apart, and have a different axial position along a length of the inflatable portion 1012.

According to some exemplary embodiments, for example as shown in FIG. 10D, at least part of each electrode is covered while keeping electrode ends facing adjacent electrodes exposed, to form ablating regions in the form of stripes, for example ablating regions 1024, 1026 and 1028. The electrodes are covered by a polymeric material, optionally an elastic polymer used for forming the balloon. In some embodiments, the covers 1030, 1032, 1034 and 1036, surround at least partially or entirely the inflatable portion 1012.

According to some exemplary embodiments, the body 1002 comprises at least one distal tip electrode located distally to the inflatable portion 1012, for example tip electrode 1038. In some embodiments, the electrode 1038 is configured to deliver stimulation and/or for pacing of the heart. Alternatively or additionally, the tip electrode 1038 is used for sensing and/or for mapping, optionally in combination with one or more of the electrodes 1006 and/or 1010.

Potential advantages of covering the electrodes may be to ensure that the electrodes remain in place during the procedure, for example during the inflation of the inflatable portion, and to form an external smooth outer surface of a tissue contacting surface of the ablation portion in order to prevent or minimize damage to tissue contacting the ablation portion due to sharp edges and therefore to improve safety of the device.

Additional potential advantages of covering the electrodes may be to control the distance between adjacent electrodes to make sure that the exposed portions of the electrodes is at the same distance from each other, for example to allow better control of the ablation depth, and to allow for a more flexible structure.

According to some exemplary embodiments, for example as shown in FIG. 10E the device 1000 is used for delivery of PFA to heart tissue. In some embodiments, the ablation portion 1004, comprising the inflatable portion 1012, is expanded within the pulmonary vein opening 1044, pushing the exposed portion of electrodes 1016 and 1018, and other electrode pairs, against the wall 1046 of the pulmonary vein. In some embodiments, at least part of the inflatable structure 1012 is located in the heart, for example in the left atrium 1048. Alternatively, the entire inflatable portion 1012 is located within the pulmonary vein 1050. In some embodiments, an electric field is delivered through the electrodes of the ablation portion 1004 to tissue contacting the exposed electrodes. In some embodiments, the electric field is delivered when the ablation portion is expanded within the body cavity and is in contact at least partly with a wall of the body cavity, for example with the wall 1046 of the pulmonary vein 1050.

According to some embodiments, a diameter of an expanded ablation portion 1004 may be in a range of from about 20-35 mm such as, for example, from about 20-25 mm, from about 25-30 mm, or from about 30-35 mm, which may be suitable for partial insertion into a pulmonary vein funnel, with about 10 mm or more of the balloon remaining within the left atrium, outside the pulmonary vein.

With regard to embodiments of FIGS. 8A-10E, it should be noted that, while these are described as having electrodes positioned on an expandable ablation portion, optionally, any of these embodiments may include electrodes, at least one of which may be partly coated with an insulative material and may have one or more windows through which the electrode is exposed. Such a configuration may potentially reduce manufacturing costs, according to some embodiments.

### Exemplary Electric Field Distribution Simulation

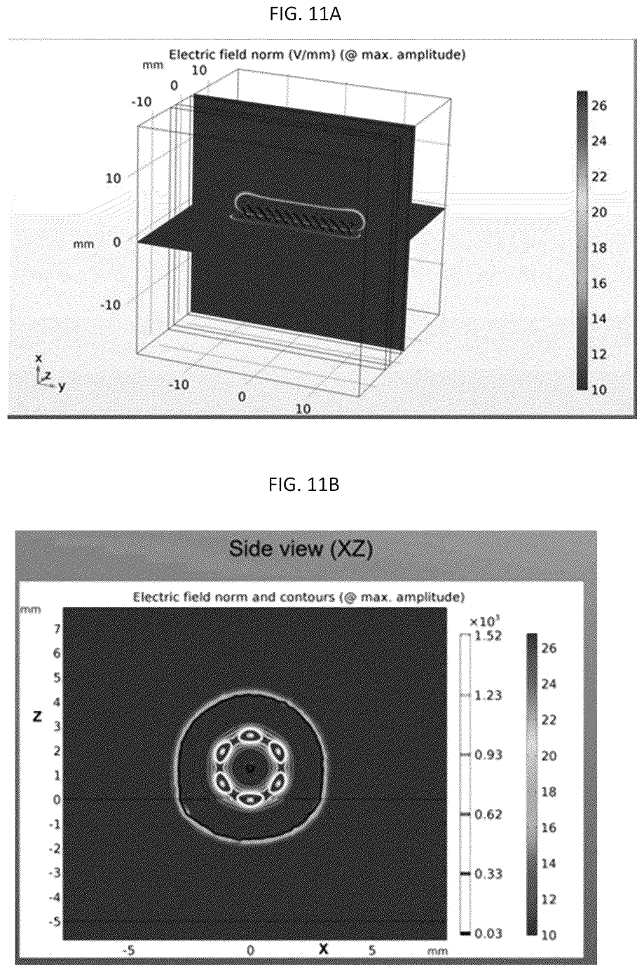

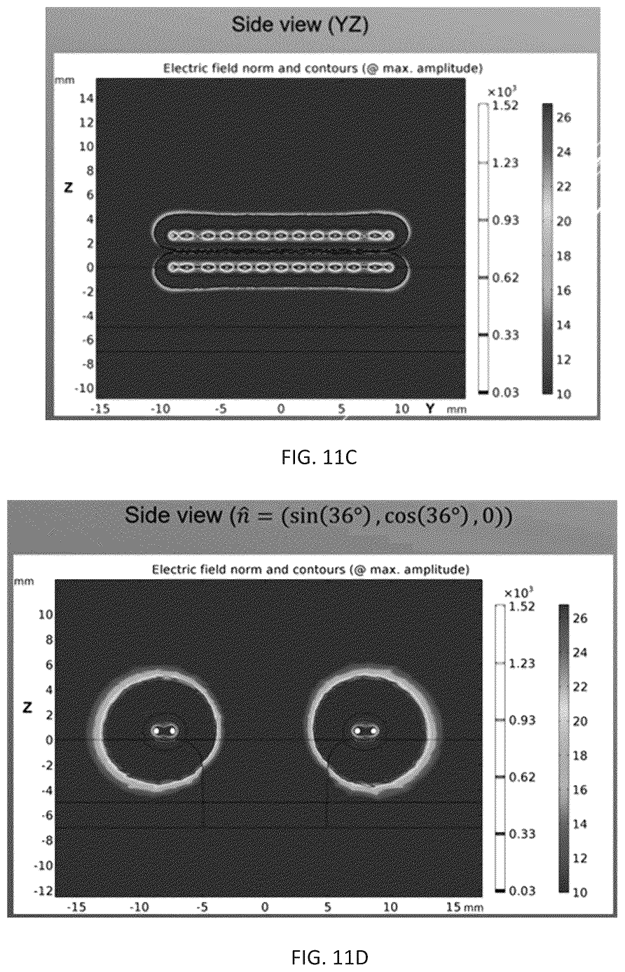

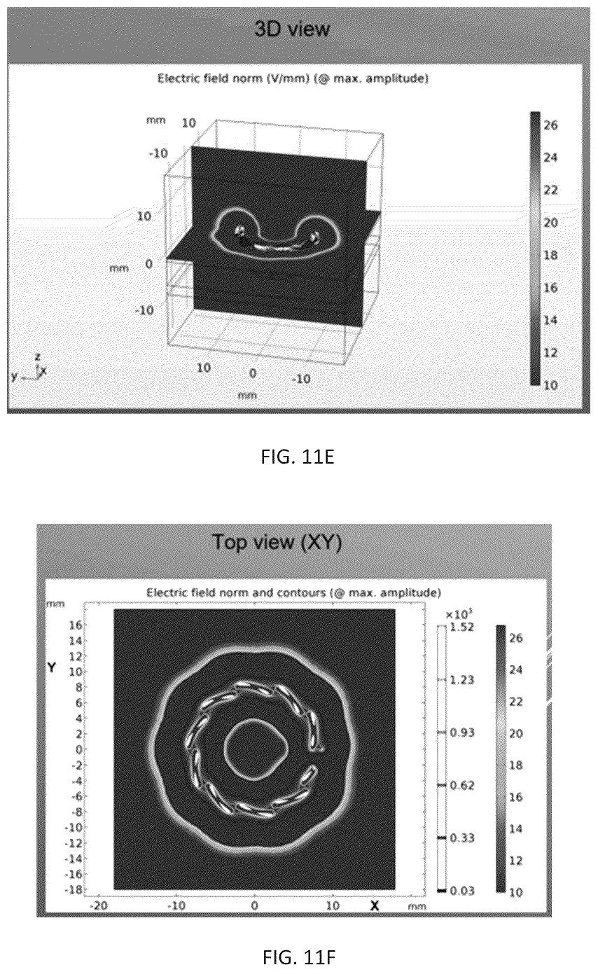

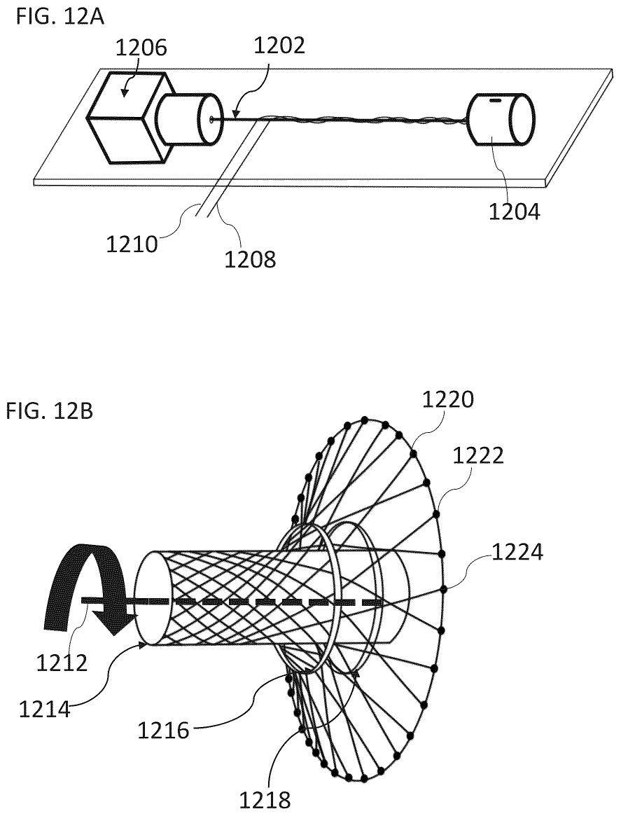

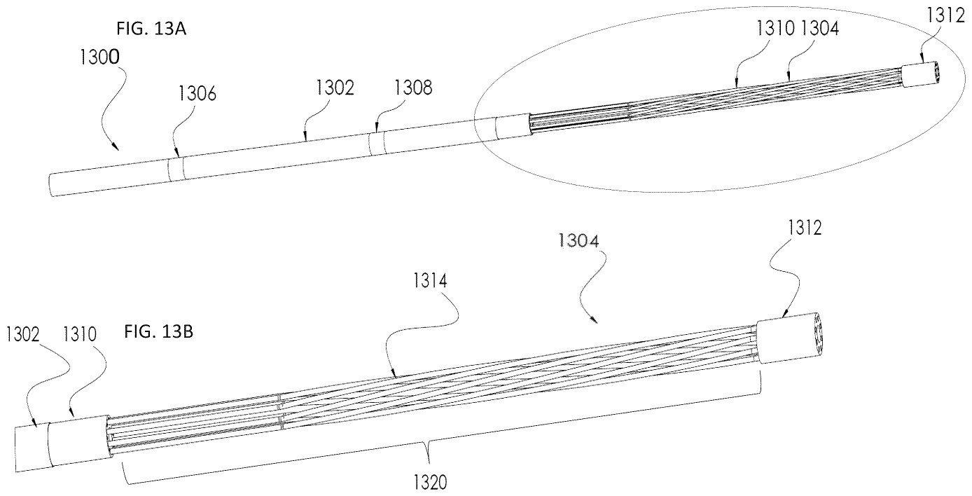

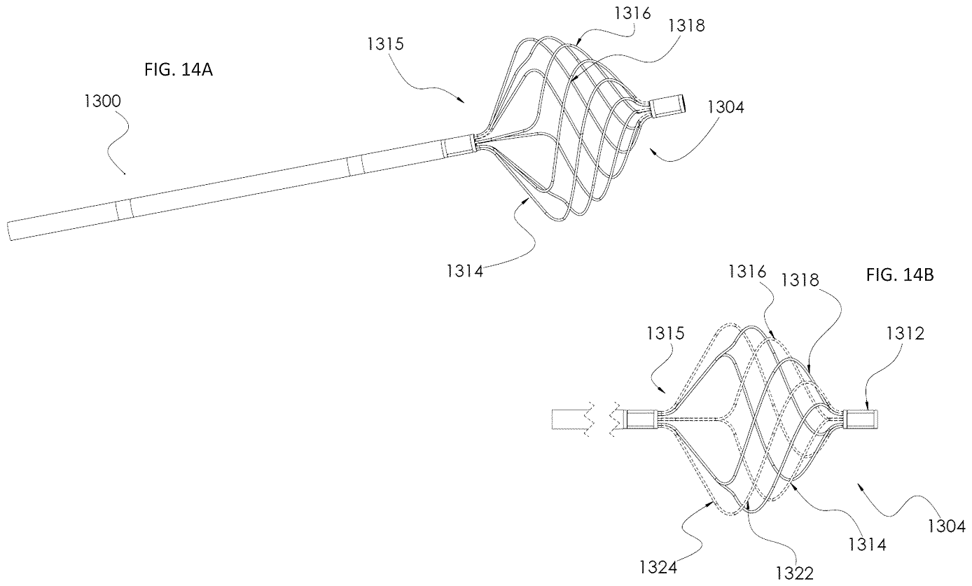

Reference is now made to FIGS. 11A-11F depicting heat maps simulating distribution of an electric field generated for example, by the ablation device of FIG. 8B, in a target tissue, according to some exemplary embodiments.

According to some exemplary embodiments, for example as shown in FIGS. 11A and 11C, when delivering an electric field to the tissue by long electrodes surrounding an ablation portion and extending along an axial length of the ablation portion or distributed along the axial length of the ablation, a penetration depth of the delivered electric field into the tissue is substantially uniform, for example deviates less than 5%, less than 3%, less than 1% or any intermediate, smaller or larger percentage value, along an axial length of the ablation portion of the device.

According to some exemplary embodiments, for example as shown in FIGS. 11B, 11C, 11D-11F, when delivering an electric field to the tissue by long electrodes surrounding an ablation portion, a penetration depth of the delivered electric field into the tissue is substantially uniform, for example deviates less than 5%, less than 3%, less than 1%, or any intermediate, smaller or larger percentage value, around the ablation portion of the device.

The simulations shown in FIGS. 11A-11C were performed for the device 800 shown in FIGS. 8A-8C having an ablation portion 804, with the following electric field parameters: voltage of 2000 volts (v), frequency of 500 kHz, pulse duration of 0.1 ms, a single pulse, and a distance between electrodes of 2 mm.

The simulations shown in FIGS. 11D-11F were performed for the device 700 shown in FIGS. 7A-7C having a lasso-shaped ablation portion 704, with the following electric field parameters: voltage of 2000 volts (v), frequency of 500 kHz, pulse duration of 0.1 ms, a single pulse, a distance between electrodes of 1 mm, and a pitch of 5 mm from adjacent long electrodes.

### Exemplary Manufacturing of Therapeutic Portion

Reference is now made to FIG. 12A depicting manufacturing of a lasso-shaped ablation portion, for example ablation portion 704 shown in FIG. 7A, according to some exemplary embodiments.

According to some exemplary embodiments, a spline 1202 is held between an anchor 1204 and a twisting device 1206. Alternatively, the spline 1202 is held by at least two twisting devices. In some embodiments, two electrode wires 1208 and 1210 are held at a fixed distance apart, and are twisted around the spline as it is rotated by the twisting device. In some embodiments, a distance and a pitch between the two electrodes is maintained during the formation. Alternatively, the distance and/or the pitch is changed during manufacturing, for example in order to generate an ablation portion with regions where the distance and/or pitch between electrodes vary.

According to some exemplary embodiments, each electrode wire of the at least two electrode wires is twisted separately around the spline.

Reference is now made to FIG. 12B depicting manufacturing of a braided mesh ablation portion having elongated electrodes, according to some exemplary embodiments.

According to some exemplary embodiments, the braided mesh structure is formed using a weaving device, comprising a mandrel 1212 coupled to an actuator, and positioned within a base structure, for example a spool 1214. In some embodiments, the braided mesh structure is formed by rotating the mandrel 1212 located within a spool 1214 while wires are disposed and twisted over the spool 1214 to form the mesh structure. In some embodiments, the wires are stationary during the rotation of the spool 1214. In some embodiments, at least one wire of the twisted wires is an electrically non-conducting wire 1222, configured to provide support for electrically conducting wires, for example for electrode wires 1220 and 1224, disposed and twisted over the spool 1214 during the spool rotation. In some embodiments, the wire 1222 is formed from a polymer material. Alternatively, the wire 1222 is formed from an electrically conducting material, for example a shape memory alloy, coated with an insulation material. In some embodiments, the electrode wires are formed from a metallic material or from a metallic alloy, configured to conduct electricity. Optionally, the electrode wires are coated with a dielectric coating.

According to some exemplary embodiments, the weaving device comprises one or more guides 1216 and 1218, for example a guide for each electrode wire, configured to direct the electrode wire to a specific location over the spool 1214. In some embodiments, a distance between the guides is predetermined, for example to maintain a specific distance between the electrode wires 1220 and 1224, as they are twisted over the spool. Optionally, the guides are shaped as rings surrounding the spool. Optionally, a spaced, for example an adjustable spacer, is coupled to the two guides 1216 and 1218, for example to maintain a target distance between the electrode wires.

According to some exemplary embodiments, the spool 1214 is rotated in a first direction, for example to twist wires in a first direction over the spool 1214. Additionally, the spool is rotated in a second, opposite direction, for example to dispose and twist additional wires of the support structure, so the wires disposed during rotation of the spool 1214 in the first direction cross and/or are interwoven with wires disposed and twisted during rotation of the spool in the second direction. Optionally, electrode wires are disposed only when rotating the spool 1224 in the first direction.

Alternatively and according to some exemplary embodiments, the spool 1214 is stationary, and at least one electrode wire, for example electrode wires 1220 and 1224, are rotated around a central axis of the spool 1214, while being twisted around the spool body and along a length of the spool body. In some embodiments, at least one holder for each electrode wire is rotated around the spool 1214 while releasing the electrode wire, for example to allow twisting of the released electrode wire around the spool 1214. In some embodiments, the at least one electrically non-conducting wire 1222 is rotated around a central axis of the spool 1214, while being twisted around the spool body and along a length of the spool body, in synchronization with the rotation and/or twisting of the at least one electrode wire.

Alternatively and according to some exemplary embodiments, both the spool 1214 and the at least one electrode wire 1220 and 1224, and/or the at least one electrically non-conducting wire 1222, rotate.

According to some exemplary embodiments, the therapeutic portion is formed from the at least two electrode wires 1220 and 1224, for example elongated electrode wires, and the at least one electrically non-conducting wire 1222. In some embodiments, when the formation of the therapeutic portion is completed, the therapeutic portion is released from the spool 1214.

Ablation Device Which may have an Ablation Portion Shaped as a Cage Having Generally Axially Extending Bars

FIG. 13A relates to an aspect of some embodiments, wherein there is depicted an ablation device 1300 having an expandable ablation portion in a collapsed state, according to some exemplary embodiments. An enlargement of the expandable ablation portion, in the collapsed state, is shown in FIG. 13B. According to some exemplary embodiments, an ablation device 1300 comprises an elongated body 1302, which is optionally flexible, for example, to allow passage of the device 1300 within blood vessels of the body, and an ablation portion 1304 at a distal portion of the body 1302. In some embodiments, the body 1302 comprises one or more electrodes, for example, ring electrodes 1306, 1308, and 1310, at different locations along the body 1302. In some embodiments, the device 1300 comprises at least one electrode optionally configured for use in a PFA procedure such as, for example, a ring electrode 1312, at a distal location relative to the expandable ablation portion 1304, or at a distal end of the ablation portion 1304.

Ablation portion 1304 is formed of a plurality of conductive electrode wires 1315 that extend axially and circumferentially around the longitudinal axis of body 1302. It should be noted that the wires are positioned such that they extend distally from the body 1302 without crossing over each other. The electrode wires each include a first, proximal portion having a substantially straight wire and a second, distal portion extending distally and circumferentially around a portion of ablation portion 1304, distal to the wire proximal portion. According to some embodiments, the distal portion of each electrode wire may begin at approximately one third to one half of the distance from the proximal end of the ablation portion and extend from that point the remaining two thirds to one half of the distance to the distal end of the ablation portion. For example, the distal portions of the electrode wires may begin at one third of the distance from the ablation portion proximal end, thereby providing a relatively large area of the ablation portion configured for contacting tissue for ablation, at the widest portion of the ablation portion and optionally at a distal portion of the ablation portion.

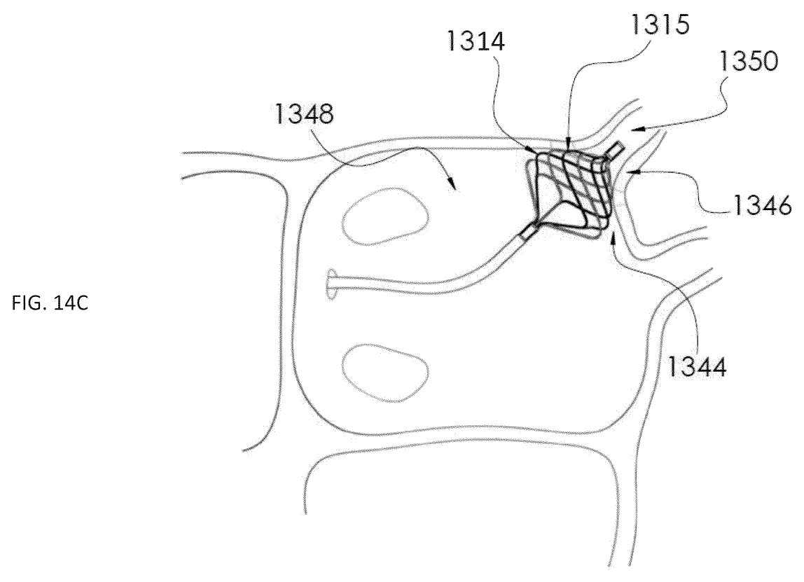

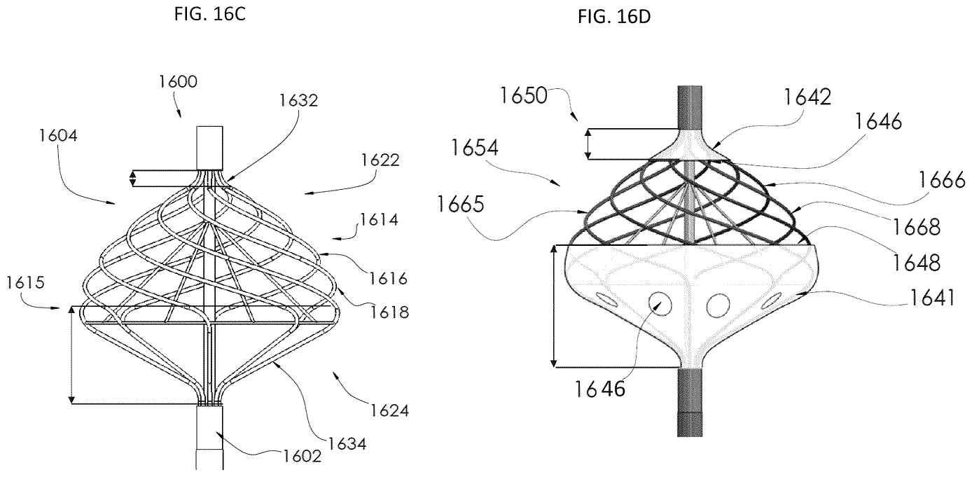

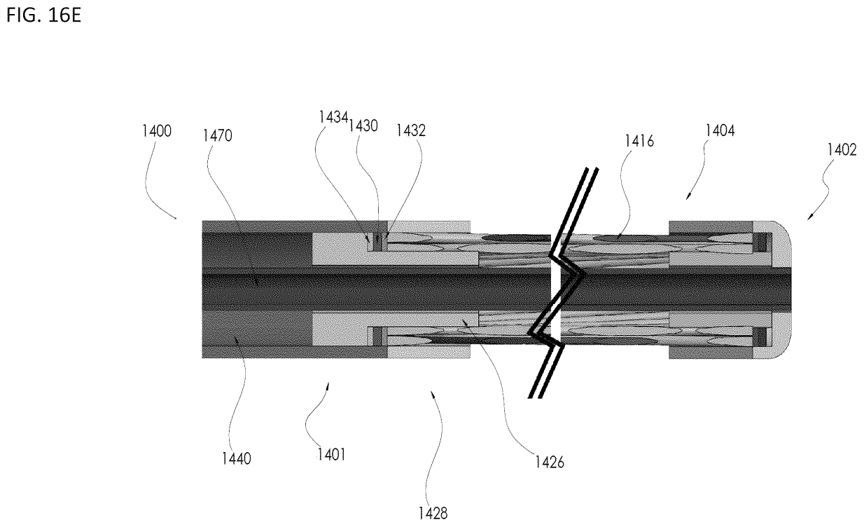

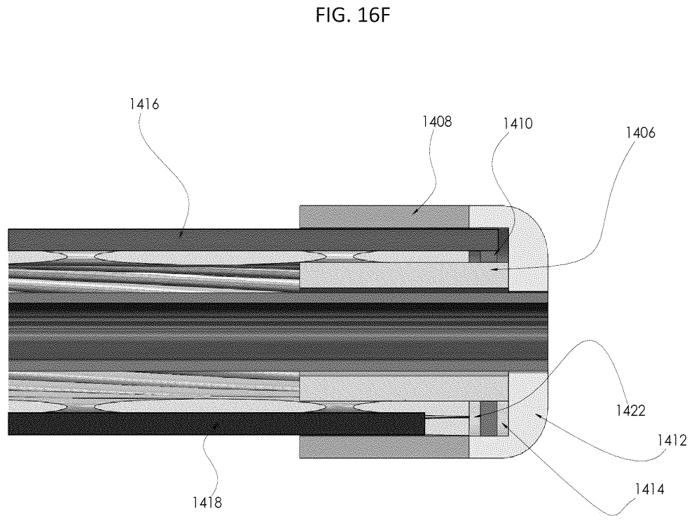

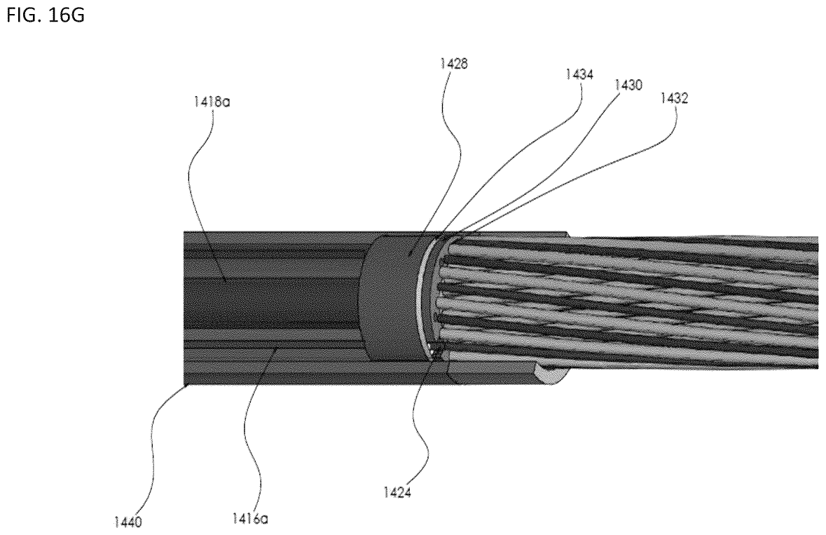

According to some exemplary embodiments, for example as shown in FIG. 13B, the expandable ablation portion 1304 has a structure 1314 configured as a cage having generally axially extending bars, formed of the plurality of conductive electrode wires 1315 such as, for example, conductive electrode wires 1316 and 1318, which extend around a circumference of the structure 1314, and extending along a length 1320 of the structure 1314. In the example shown in FIGS. 14A-B, ablation portion 1304 includes eight electrode wires (four positive electrode wires alternating in position with four negative electrode wires). It will be appreciated by persons skilled in the art that, alternatively, if desired, structure 1314 may include any preselected number of electrodes wires. For example, structure 1314 may include from four to ten electrode wires. The electrode wires 1315 are positioned at a fixed distance apart, so as to perform uniform and consistent ablation (electroporation) of tissue such as, for example, heart tissue, located between adjacent electrodes, according to some embodiments. The distance between electrode wires may be maintained by the resilience of the electrode wires and/or by the provision of tensioning structures, as discussed herein, for example, with regard to FIGS. 16A-B, according to some embodiments. The shapes of the electrodes 1315, in particular extending radially around the ablation portion 1304, facilitates the ablation of an entire circumference of a pulmonary vein, for example, if desired, according to some embodiments.

While some embodiments may be described herein as having first and second electrodes or electrode sections being positive and negative, respectfully, it should be appreciated by persons skilled in the art that this description may be for simplicity and that, alternatively, such first and second electrodes or electrode sections may be understood as having respective first and second polarities which may change during use. Also, more than two polarities may be used and/or some electrodes may be electrified at a different, possibly overlapping time, than other electrodes.

In some embodiments, when the structure 1314 is expanded, for example, as shown in FIG. 14A, the two or more electrode wires surrounding the structure are pushed against a tissue surface of a body cavity, in the same manner or in a similar manner to that discussed herein with regard to the embodiments of FIG. 8E, and as discussed further herein.

According to some exemplary embodiments, the electrodes wires 1315 may be substantially parallel to each other along most of their length, when the ablation portion 1304 is in a collapsed state, with a deviation in an average distance between adjacent electrodes of up to 10% of the resting distance between adjacent electrodes, along the electrode lengths in the ablation portion 1304, when the ablation portion is in a collapsed state and when the ablation portion is in an expanded state. Alternatively, a deviation in an average distance between adjacent electrodes may be up to 20% of the average distance between adjacent electrodes along the electrode lengths in the ablation portion when in the collapsed state and when the ablation portion is in an expanded state. Optionally, the average deviation distance between adjacent electrodes in an expanded state may be from 30-40%, up to 8%, up to 5%, up to 3%, up to 1% or any intermediate, smaller or larger deviation percentage in average deviation distance between adjacent electrodes in the ablation depth along the ablation line, according to some embodiments. In some embodiments, a distance between adjacent electrodes of the ablation portion 1304, for example electrodes 1316 and 1318, may be up to 5 mm, for example up to 4 mm, up to 3 mm, up to 2 mm, up to 1 mm, up to 0.5 mm or any intermediate, smaller or larger distance between the electrodes.

According to some embodiments, the electrode wires 1315 may each include a distal portion 1322 and a proximal portion 1324. According to some embodiments, optionally, proximal portions 1324 of wires 1315 may be coated with an insulative material. Distal portions 1322 of wires 1315 may be twisted such that a distance between adjacent wires may remain substantially consistently parallel to each other along most of their length, as discussed herein. Proximal portions 1324 of wires 1315 may be substantially straight, such that length of the ablation portion 1314 may be as short as possible when structure 1314 is in the collapsed state (FIG. 13A-B). A potential advantage of maintaining the electrode distal portions 1322 substantially parallel to each other may be to ensure that the electric field depth and the ablation line remains consistent or substantially consistent with deviation in an average electric field depth of from 30-40%, from 20-30%, from 10-20%, from 5-10%, less than 5%, less than 3%, less than 1% or any intermediate, smaller or larger deviation percentage in the ablation depth along the ablation line, according to some embodiments.

According to some exemplary embodiments, the conductive electrode wires 1315 may be formed of self-expandable material including, for example, nitinol. When in a collapsed state, such as shown in FIG. 13A, at least the ablation portion 1304 of device 1300 may be temporarily housed in a delivery sheath (not shown) for introduction into the left atrium 1348 and then into a blood vessel such as, for example, the pulmonary vein 1350, using the body 1302 as a guide. The ablation portion 1304 may be deployed from the sheath when at or near a location to be ablated, as discussed herein, according to some embodiments. Once deployed, the self-expandable wires may assume the expanded state shown in FIGS. 14A-C, wherein the ablation portion may assume a configuration having a diameter of from 20-30 mm such as, for example, approximately 25 mm, according to some embodiments. Wire electrodes may have a diameter of at most 2 mm, according to some embodiments.

According to some embodiments, each wire may have a rectangular cross-sectional profile, with a shorter side of the rectangular profile positioned radially inward relative to the body longitudinal axis, such that the wire can bend radially outward (e.g., when the ablation portion expands) but does not bend substantially in a direction toward an adjacent wire.

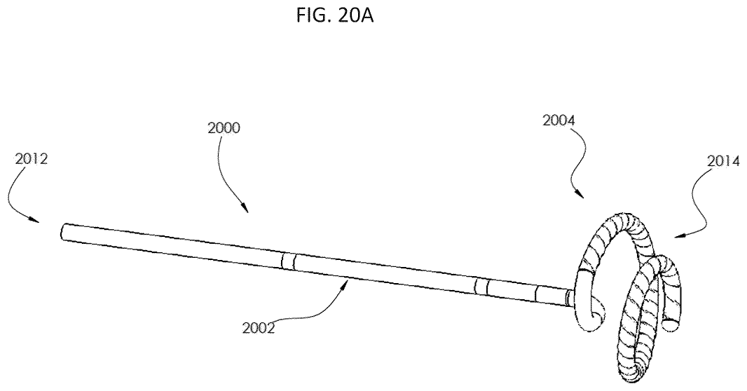

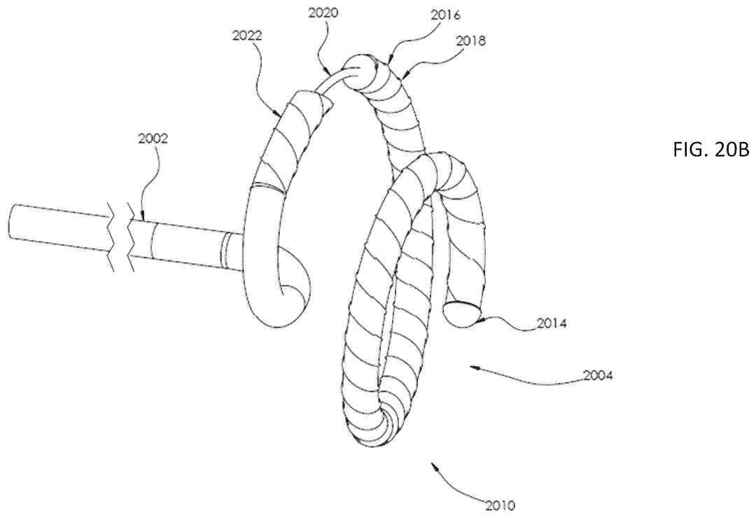

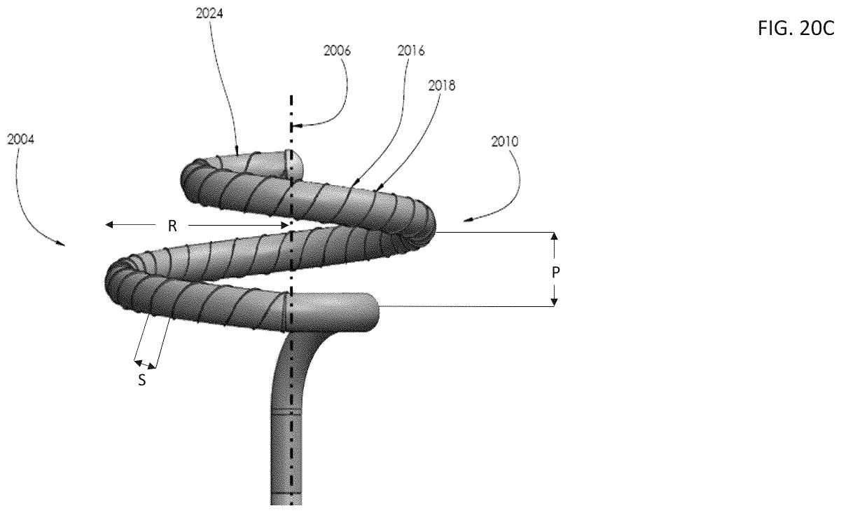

Optionally, the at least one axially and circumferentially extending electrode 1315 of the structure 1314 may include a plurality of axially extending spirally-shaped electrodes, for example, forming a structure shaped as a double helix, according to some exemplary embodiments. Optionally, at least one axially and circumferentially extending electrode 1315 of the structure 1314 may be provided with a plurality of electrode wires wound around the electrode and attached thereto, optionally in a spiral configuration, as discussed herein, for example, with regard to FIGS. 20A-C. In this manner, the at least one axially and circumferentially extending electrode may provide structure for the ablation portion, while the electrode wires would provide the ablation component, according to some exemplary embodiments.

According to some exemplary embodiments, the at least one electrode 1312 or any other electrode optionally located distally to the structure 1314 may be used for pacing such as, for example, pacing of heart tissue. In some embodiments, one or more of the electrodes, for example ring electrodes 1306, 1308 and 1310 (FIG. 13A), may optionally be used for at least one of delivery of the PFA, pacing, and mapping of signals such as, for example, electric signals, through heart tissue, according to some embodiments. Pacing may be important in checking for electrical leaks after ablation and/or to find conduction pathways before ablation and/or to overpace the atrium so that it does not contract substantially during the ablation procedure.

According to some exemplary embodiments, the device 1300 may optionally be used for delivery of PFA to heart tissue as discussed herein. In some embodiments, the ablation portion 1304, comprising the structure 1314, may be expanded within the pulmonary vein opening 1344, pushing the electrodes 1315 against the wall 1346 of the pulmonary vein 1350. In some embodiments, at least part of the expanded structure 1314 may be located in the heart, for example in the left atrium 1348. Alternatively, the entire structure 1314 may be located within the pulmonary vein 1350. In some embodiments, an electric field may be delivered through the electrode wires 1315 of the ablation portion 1304 to tissue contacting the electrodes, when the ablation portion is expanded within the body cavity and is in contact at least partly with a wall of the body cavity, for example with the wall 1346 of the pulmonary vein 1350. According to some embodiments, adjacent electrode wires such as, for example, electrode wires 1316 and 1318, may contact tissue of the wall 1346 of the pulmonary vein 1350 in order for ablation to be performed between the electrode wires. The particular twisted structure of the ablation portion 1304, according to some embodiments, may allow ablation of tissue along an inner circumference or a portion of an inner circumference of the pulmonary vein.

In some embodiments, the expanded structure 1314 may be flexible enough, for example, due to having fewer than two cross members per mm and/or due to the electrode wires not crossing over each other, to adapt to anatomy of the veins, whether the veins have a bifurcation at the PV/atrial junction, stenosis, an uneven surface, or a smaller than usual opening. In some embodiments, the structure may be small enough to enter any PV and expand to an exact size and create contact between the electrodes and the PV circumference, at the contact region between the expanded structure and the PV wall.

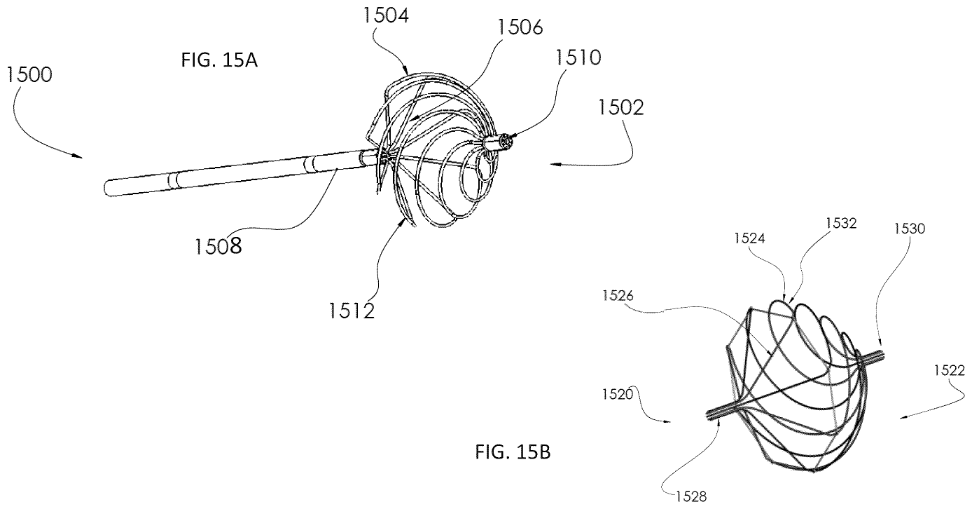

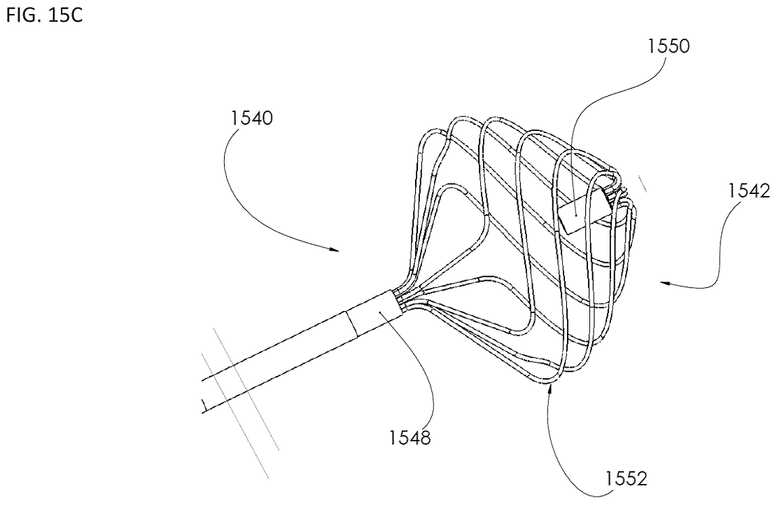

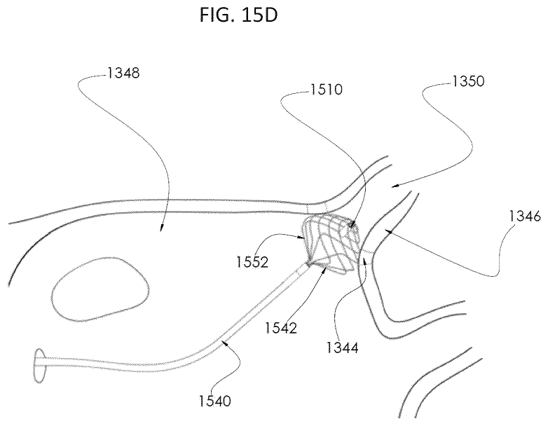

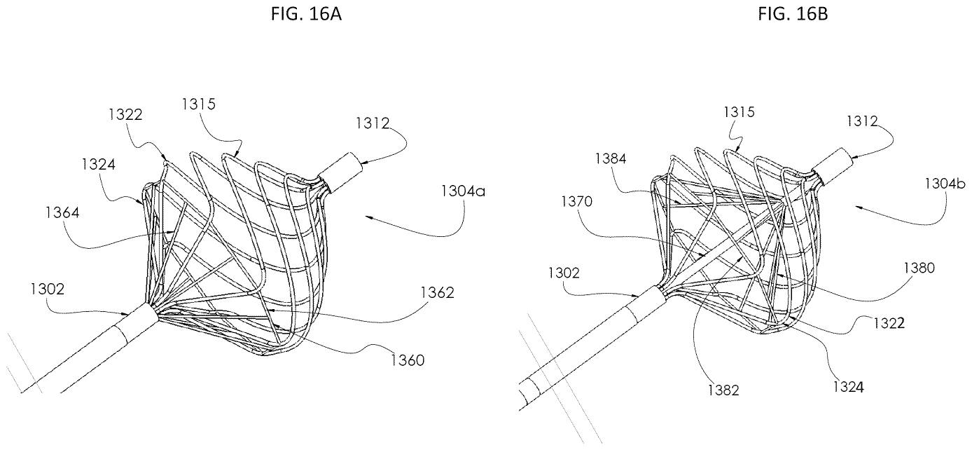

FIGS. 15A-C are schematic illustrations of expandable ablation portions 1502, 1522, and 1542 of ablation devices 1500, 1520, and 1540, respectively, according to alternative exemplary embodiments. Some components of ablation devices 1500, 1520, and 1540 may be identical or similar in structure and function to components of ablation device 1300, and may not be described again herein. However, as seen in FIG. 15A, ablation portion 1502 of device 1500 includes electrodes wires 1512 each having a distal portion 1504 and a proximal portion 1506. It may be noted that proximal portions 1506 of electrode wires 1512 each extend radially outward from body 1508, and that distal portions 1504 of electrode wires 1512 curve radially inwardly and distally toward ring electrode 1510, optionally forming a spiral shape, according to some embodiments.

As seen in FIG. 15B, ablation portion 1522 of device 1520 includes electrodes wires 1532 each having a distal portion 1524 and a proximal portion 1526. It may be noted that proximal portions 1526 of electrode wires 1532 each extend radially outward and distally from body 1528, and that distal portions 1524 of electrode wires 1532 curve radially inwardly and distally toward ring electrode 1530, optionally forming a spiral shape, according to some embodiments.

FIG. 15C is a schematic illustration of yet a further embodiment of the embodiment, wherein an ablation portion 1542 of device 1540 includes electrodes wires 1552 each having a substantially spiral shape extending from body 1548 to ring electrode 1550, optionally forming a spiral shape, according to some embodiments. Additionally, it should be noted that in this embodiment ring electrode does not extend distally relative to electrode wires 1552 (as does, for example, ring electrode 1510 in device 1500). Rather, ring electrode 1510 is formed on an inner portion of ablation portion 1542, radially inward relative to electrode wires 1552. This potentially allows for the ablation of tissue using a distal portion of the ablation portion 1542.

FIG. 15D is a schematic illustration showing the ablation device 1540 of FIG. 15C, shown in an expanded state, within an opening of a pulmonary vein ostium 1344, according to some exemplary embodiments. Some components of ablation device 1540 may be identical or similar in structure, function, and disposition within the left atrium 1348 or pulmonary vein 1350 to those of ablation device 1300 (FIG. 14C), and may not be described again herein. It can be seen, however, that in device 1540 ring electrode 1510 is disposed radially inward relative to electrode wires 1552, thereby optionally allowing contact between a distal portion of the ablation portion 1542, for ablation of tissue using the distal portion of the ablation portion 1542, as discussed herein.

According to some embodiments, an ablation portion of a device described with reference to any of FIGS. 13A-15D, when in an expanded state, may have a diameter, at its widest point, selected from the following ranges: from about 10 to about 15 mm, from about 15 to about 25 mm, and from about 25 to about 35 mm, such as, for example, 10 mm, 15 mm, 20 mm, 25 mm, 30 mm, or 35 mm or intermediate values.

FIGS. 16A-B relate to an aspect of some embodiments, wherein expandable ablation portions may include an additional tensioning wire, which will be discussed in detail herein. Expandable ablation portions 1304a and 1304b are shown in FIGS. 16A and 16B, respectively, wherein the expandable ablation portions are identical or similar to ablation portion 1304 of FIG. 14B, having identical or similar structure and function may not be discussed again herein. However, ablation portion 1304a (FIG. 16A) is provided with at least one additional tensioning wire 1360, which may be located inside or outside of ablation portion 1304a, and which may be tied or otherwise attached to each of electrode wires 1315, at proximal portions thereof. The at least one additional tensioning wire 1360 may include radially extending portions 1362 which extend between adjacent wires 1315 and/or portions 1364 extending distally, from body 1302 to wires 1315. The at least one additional wire 1360 may apply tension to the electrode wires 1315, thereby urging the electrode wires 1315 to desired positions within the ablation portion 1304a and/or relative to each other and/or preventing the electrode wires from being moved relative to each other and/or being deformed inadvertently and/or contacting each other, according to some embodiments. The at least one additional wire 1360 may allow the electrode wires 1315 to maintain their form and/or relative positions within the ablation portion 1304a, even when the ablation portion is in a collapsed state.

Ablation portion 1304b (FIG. 16B) is optionally provided with a central rod 1370 extending axially, from body 1302 to distal electrode 1312, and at least one additional wire 1380, which may be tied or otherwise attached to each of electrode wires 1315, at proximal portions thereof. The at least one additional wire 1380 may include radially extending portions 1382 which extend between adjacent wires 1315 and/or portions 1384 extending distally, from rod 1370 to wires 1315 or to portions 1382. The at least one additional wire 1380 may apply tension to the electrode wires 1315, thereby urging the electrode wires 1315 to desired positions within the ablation portion 1304b and/or relative to each other and/or preventing the electrode wires from being moved relative to each other and/or being deformed inadvertently and/or contacting each other. The at least one additional wire 1380 may allow the electrode wires 1315 to maintain their form and/or relative positions within the ablation portion 1304b, even when the ablation portion is in a collapsed state.

Optionally, any of the embodiments of FIGS. 13A-16B may include an ablation portion having at least distal portion of the electrode wires provided with undulations, zigzags, or other curved shapes, thereby increasing the area between adjacent electrodes that will be ablated, according to some embodiments.

It may be noted that, while some embodiments such as, for example, that of FIG. 8D may include non-conductive material between electrode wires, each of the embodiments of FIGS. 13A-16B may have no such non-conductive material between electrode wires, thereby providing a potential advantage of allowing the device to ablate large sections in the left atrium of the heart without being limited by the extent of expansion of material of a balloon.

Additionally, it may be noted that, in the embodiments of FIGS. 16A-B, portions of the structure, namely tensioning wires 1360/1382, portions 1362 and 1364/1382 (FIGS. 16A-B), and central rod 1370 (FIG. 16B) are positioned radially inward relative to the electrode wires 1315. They will not, therefore, contact tissue when the ablation portion is expanded. In contrast, in the embodiment of FIG. 14A, for example, optionally all wires 1315 of the structure may contact tissue when the ablation portion is expanded.

FIG. 16C relates to an aspect of some embodiments, wherein there is depicted an ablation device 1600 having an expandable ablation portion 1604 in an expanded state, according to some exemplary embodiments. Some components of ablation portion 1604 may be identical or similar in structure and function to corresponding components of ablation portion 1304b (FIG. 16B), and may not be discussed herein in detail.

According to some exemplary embodiments, ablation portion 1604 is formed of a plurality of conductive electrode wires 1615 such as, for example conductive wires 1616 and 1618, which may be identical or similar in structure and function to conductive electrode wires 1315 (e.g., as shown in FIG. 16B). In the example shown in FIG. 16C, ablation portion 1604 is shown in an expanded state. Ablation portion 1604 may include eight electrode wires (four positive electrode wires alternating in position with four negative electrode wires), according to some exemplary embodiments. It will be appreciated by persons skilled in the art that, alternatively, if desired, structure 1614 may include any preselected number of electrodes wires. For example, structure 1614 may include from four to ten electrode wires. According to some exemplary embodiments, the electrode wires 1615 are positioned at a fixed distance apart, which may be identical or similar in structure and function to electrodes wires 1315 (FIG. 16B).

According to some exemplary embodiments, the electrodes wires 1615 may be substantially parallel to each other along most of their length, when the ablation portion 1604 is in a collapsed state (not shown) and when the ablation portion is in an expanded state (FIG. 16C), in a manner identical or similar to the collapsed state and expanded state discussed herein with regard to the embodiments of FIGS. 13A-16B.

According to some embodiments, the electrode wires 1615 may each include a distal portion 1622 and a proximal portion 1624. According to some embodiments, optionally, distal portions 1622 and/or proximal portions 1624 of wires 1615 may be provided with an insulating material 1632 and 1634, respectively, in the form of a coating or plating or cover or sleeve such as, for example, a heat shrink sleeve, having an insulating property. For example, the insulating material 1632 and/or 1634 may be formed of an enamel, ePTFE (polytetrafluoroethylene), polyimide, FEP (fluorinated ethylene propylene), PFA (perfluoroalkoxy alkanes), or any other suitable material that may insulate the distal/proximal portions of the electrode wires 1615. According to some exemplary embodiments, the insulating material may be applied to the distal portions 1622 and/or to the proximal portions 1624 by dipping, spraying, electrocoating, or any other suitable method.

According to some exemplary embodiments, insulating material 1632 may cover up to 15% of the distal portions 1622 of electrode wires 1615, and insulating material 1634 may cover up to 50% of the proximal portions 1624 of electrode wires 1665.

The provision of an insulating material 1632 and/or 1634 on ablation portion 1604 may ensure that portions of electrode wires 1615 that are to contact tissue to be ablated, i.e., portion of the electrode wires 1615 that are not covered by insulating material, are maintained equidistant from each other, according to some exemplary embodiments.

It will be appreciated by persons skilled in the art that, optionally, provision of an insulating material such as discussed herein with regard to FIG. 16C may optionally be applied to any of the ablation portions discussed herein.

FIG. 16D illustrates a device 1650 having an ablation portion 1654 including electrode wires 1665, according to some exemplary embodiments. Device 1650 may be identical or similar in structure and function to device 1600 (FIG. 16C), and corresponding components may not be discussed again herein in detail.

According to some exemplary embodiments, electrode wires 1665 may be provided with a distal cover 1642 on a distal portion 1646 of electrode wires 1665 and/or a proximal cover 1644 on a proximal portion 1648 of electrode wires 1665. According to some exemplary embodiments, distal cover 1642 and/or proximal cover 1644 may be in the form of a liner or sheet formed of a polymer material such as, for example, an enamel, ePTFE (polytetrafluoroethylene, or polyimide. Optionally, the distal cover 1642 and proximal cover 1644 are biocompatible, non-thrombogenic, and/or microporous, according to some exemplary embodiments.

According to some exemplary embodiments, distal cover 1642 may cover up to 15% of the distal portions 1646 of electrode wires 1665, and proximal cover 1644 may cover up to 50% of the proximal portions 1648 of electrode wires 1665. Each of distal cover 1642 and proximal cover 1644 may be positioned radially outside the electrode wires 1665, according to some exemplary embodiments. Alternatively, according to some exemplary embodiments, distal cover 1642 and/or proximal cover 1644 may be positioned both radially outside and radially inside the electrode wires 1665.

Optionally, the distal cover 1642 and/or the proximal cover 1644 may include apertures 1646 which may be sized and shaped to allow blood to flow therethrough. For example, the apertures 1646 may be circular or oval. The apertures 1646 may each have a diameter in a range of from about 0.5-6.0 mm such as, for example, from about 0.5-1 mm, from about 1-3 mm, from 2-4 mm, or from about 4-6 mm. Any suitable number of apertures 1646 may be provided such as, for example, 20 apertures. Provision of apertures 1646 may prevent blood from coagulating and the formation of blood clots during an ablation procedure, according to some exemplary embodiments.

The provision of a distal cover 1642 and/or a proximal cover 1644 on ablation portion 1654 may ensure that portions of electrode wires 1665 that are to contact tissue to be ablated, i.e., those portions of electrode wires 1665 not covered by distal cover 1642 and proximal cover 1644, are equidistant from each other, according to some exemplary embodiments.

It will be appreciated by persons skilled in the art that, optionally, the distal cover 1642 and/or the proximal cover 1644 may optionally be applied to any of the ablation portions discussed herein, according to some exemplary embodiments.

Optionally, according to some exemplary embodiments, in order to increase the conductivity of the electrode wires of any of the embodiments discussed herein, the electrode wires may be formed of a nitinol core plated or coated with a suitable material such as, for example, gold, a platinum alloy, a palladium alloy, silver, tantalum, or any other combination of materials that is conductive and biocompatible. The ratio of nitinol material to plating or coating material may be in a range of from about 10:90 to about 90:10 such as, for example, from about 10:90 to about 30:70, from about 30:70 to about. 50:50, from about 50:50 to about 70:30, or from about 70:30 to about 90:10.

FIGS. 16E-G are schematic illustrations of portions of an expandable ablation device 1400, in a collapsed state, according to some exemplary embodiments. It should be noted that FIGS. 16E-G are exemplary only, and are intended to illustrate how the various components of the ablation device 1400 may be assembled, while ensuring that the electrodes do not contact each other. It is to be understood that numerous variations of the described assembly will be understood by persons skilled in the art.

With specific reference to FIG. 16E there is shown a portion of ablation device 1400 including an axially extending body 1440 and an ablation portion 1404, as discussed herein. Components of ablation portion 1404 may be identical or similar in structure and/or function to components of ablation portion 1304 (FIG. 13B) and may not be described again herein.

Ablation portion 1404 has a proximal end 1401 and a distal end 1402 and may include a central rod 1470 extending axially along the ablation portion, according to some exemplary embodiments. According to some exemplary embodiments, ablation portion 1404 may also include a plurality of conductive electrode wires 1416 having a first polarity and a plurality of conductive electrode wires 1418 having a second polarity, as discussed herein. Central rod 1470 and wires 1416 and 1418 may extend axially along device 1400 and terminate at ablation portion distal end 1402, as will be discussed further below, according to some exemplary embodiments.

With reference to FIG. 16F, it is shown that ablation portion distal end 1402 includes an end cap 1412. End cap 1412 may be formed of an electrically insulative material such as, for example, PEEK (polyether ether ketone), PS (polystyrene), PP (polypropylene), PVC (polyvinyl chloride), PC (polycarbonate), PU (polyurethane), PET (polyethylene terephthalate), or a PIs (polyimides), according to some exemplary embodiments. A distal metal plate 1410, or other electrically conductive plate, formed of, for example, nitinol, stainless steel, gold, silver, Ta (tantalum), or platinum, may be positioned at ablation portion distal end 1402, within end cap 1412, optionally attached to the end cap by an adhesive 1414, according to some exemplary embodiments. Optionally, a distal plastic plate 1422, or other electrically insulative plate formed of, for example, kapton, PS (polystyrene), PP (polypropylene), PVC (polyvinyl chloride), PC (polycarbonate), PU (polyurethane), PET (polyethylene terephthalate), or PIs (polyimides) may be positioned at ablation portion distal end 1402, proximal to distal metal plate 1410. According to some exemplary embodiments, a distal insulative tube 1406, formed of, for example, PEEK, PS (polystyrene), PP (polypropylene), PVC (polyvinyl chloride), PC (polycarbonate), PU (polyurethane), PET (polyethylene terephthalate), or PIs (polyimides) may be positioned at ablation portion distal end 1402, radially outside central rod 1470, and may extend distally through distal plastic plate 1422 and distal metal plate 1410. An insulative ring 1408 formed of, for example, PEEK, PS (polystyrene), PP (polypropylene), PVC (polyvinyl chloride), PC (polycarbonate), PU (polyurethane), PET (polyethylene terephthalate), or PIs (polyimides), may be positioned at ablation portion distal end 1402, radially outside wires 1416 and 1418, according to some exemplary embodiments.

According to some exemplary embodiments, wires 1416 (having the first polarity) may contact distal metal plate 1410 at their distal ends, such that they are electrically in contact with distal metal plate 1410. Optionally, wires 1416 may be welded to distal metal plate 1410 at their distal ends, according to some exemplary embodiments. Wires 1418 (having the second polarity) may terminate at their distal ends at a distance from distal metal plate 1410, between distal insulating tube 1406 and distal insulating ring 1408, such that the wires 1418 are electrically isolated from each other and from other components of ablation portion 1404, according to some exemplary embodiments.

With reference to FIG. 16G, it is shown that ablation portion proximal end 1401 includes a proximal insulating ring 1428 formed of any insulative material such as, for example, PEEK, PS (polystyrene), PP (polypropylene), PVC (polyvinyl chloride), PC (polycarbonate), PU (polyurethane), PET (polyethylene terephthalate), or PIs (polyimides). A proximal metal plate 1430, or other electrically conductive plate formed of an electrically conductive material such as, for example, nitinol, stainless steel, gold, silver, Ta (tantalum), or platinum, may optionally be attached to the proximally insulating ring 1428 by an adhesive 1434, according to some exemplary embodiments. Optionally, a proximal plastic plate 1432, or other electrically insulative plate, may be positioned distal to proximal metal plate 1430, according to some exemplary embodiments. A proximal insulative tube 1426 (FIG. 16E), formed of, for example, PEEK, PS (polystyrene), PP (polypropylene), PVC (polyvinyl chloride), PC (polycarbonate), PU (polyurethane), PET (polyethylene terephthalate), or PIs (polyimides), may be positioned at ablation portion proximal end 1401, radially outside central rod 1470, and may extend distally through proximal metal plate 1430 and proximal plastic plate 1432, according to some exemplary embodiments.

Wires 1418 (having the second polarity) may contact proximal metal plate 1430 at their proximal ends, such that they are electrically in contact with the proximal metal plate 1430, according to some exemplary embodiments. Optionally, wires 1418 may be welded to proximal metal plate 1430 at their proximal ends. Wires 1416 (having the first polarity) may terminate at their proximal ends at a distance from proximal metal plate 1430, between proximal insulating tube 1426 and proximal insulating ring 1428, such that wires 1416 are electrically isolated from each other and from other components of ablation portion 1404, according to some exemplary embodiments.

One wire 1418a, of wires 1418 (having the second polarity), may extend through proximal plastic plate 1432 and proximal metal plate 1430 and may extend proximally along body 1440, toward a handle located at a proximal end (not shown) of device 1400, according to some exemplary embodiments. One wire 1416a, of wires 1416 (having the first polarity), may extend through an opening 1424 in proximal metal plate 1430, thereby being isolated from proximal metal plate 1430, and may extend proximally within body 1440, toward the handle (not shown), according to some exemplary embodiments.

The proximal end of each of wires 1418a and 1416a may be connected to a conductive wire, and the conductive wires may extend proximally toward the handle (not shown), according to some exemplary embodiments.

Ablation Device Including an Ablation Portion having Electrode Sections

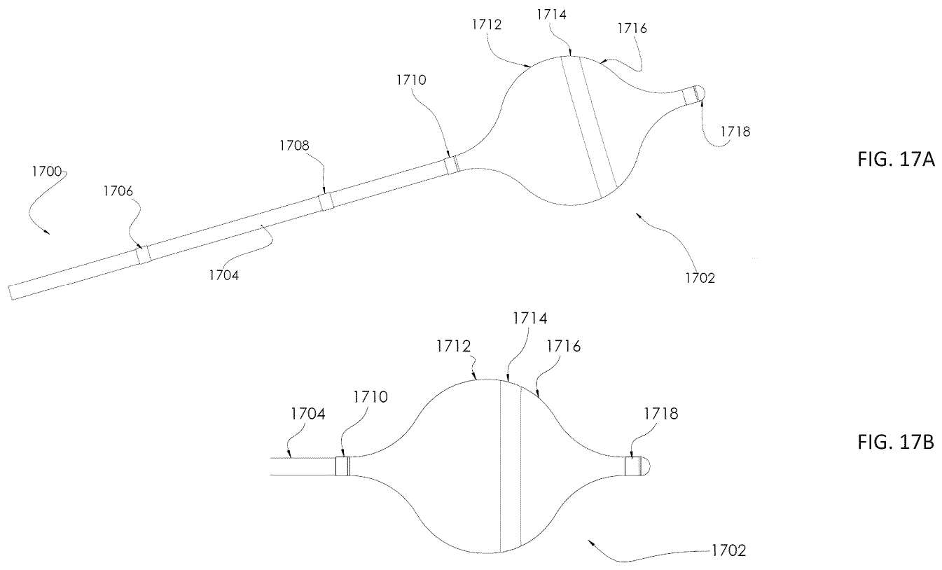

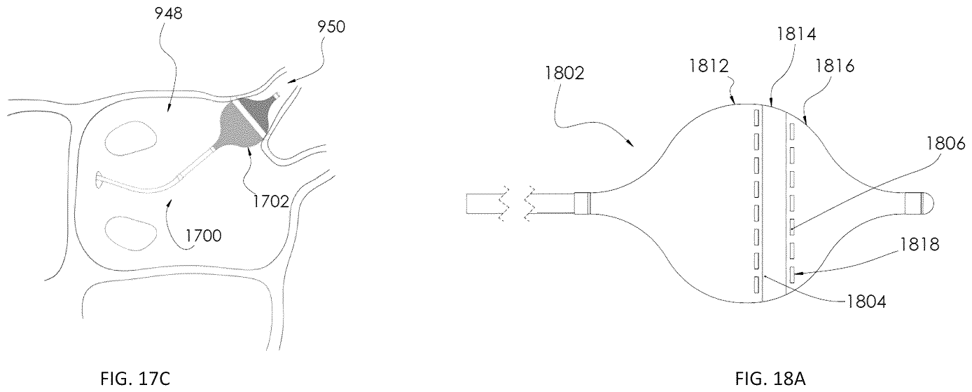

FIG. 17A relates to an aspect of some embodiments, wherein an ablation device 1700 has a body 1704 and an ablation portion 1702 in an expanded state, according to some exemplary embodiments. Since ablation device 1700 includes components that may be identical or similar in structure and function to those of other embodiments discussed herein, for example, ablation device 800 (FIG. 8D), those components may not be described again herein. It should be appreciated by persons skilled in the art that, optionally, other components may be utilized in ablation device 1700 such as, for example, other components discussed herein and/or other designs.

FIG. 17B shows an enlargement of the ablation portion 1702 of ablation device 1700 in an expanded state, according to some exemplary embodiments. As opposed to other embodiments discussed herein such as, for example, that of FIG. 9E, where electrodes may be disposed on an expandable mesh structure, in the embodiment of FIG. 17B the ablation portion 1702 includes a balloon or other selectively inflatable portion having formed thereon specifically shaped portions which are coated, electroplated, metalized, or printed on sections thereof with electrically conductive material. The electrically conductive material may be, for example, a combination of an electrically conductive material and a polymer having any suitable ratio such as, for example 50% conductive material and 50% polymer. For example, the two materials can be stainless steel or gold combined with Pebax® or silicone or any other suitable material combination. The coating may be formed by any suitable method such as, for example, by a process of electrospinning nanofibers, where a balloon may be placed on a spinning mandrel and a needle above the mandrel may dispense a combination of materials in a molten state, which may be sprayed onto the balloon surface. The combination of molten materials will form the electrode section(s) with the balloon inside the electrode section(s).

The combination of electrically conductive material and polymer may allow the electrode portion to be both conductive and flexible and, therefore, more resistant to abrasion or scratches. This may be an important feature since, for example, an ablation portion such as a balloon ablation portion having electrode sections may be inserted into an introducer, i.e., a tube or funnel-shaped device, optionally made of, for example, polypropylene or PTEF (polytetrafluoroethylene), which may reduce a diameter of the collapsed balloon to that of an inner diameter of the delivery sheath. The balloon may rub against the inner surface of the delivery sheath which may be made of or lined with, for example, PTFE. After deployment, when the balloon is collapsed and retracted back into the delivery sheath, the balloon may rub against the distal edge of the delivery sheath which may be formed of, for example, Pebax® or another soft polymer.

In the embodiment shown, ablation portion 1702 may include a positive electrode section 1712 (also referred to as “positive electrode 1712”) and a negative electrode section 1716 (also referred to as “negative electrode 1716”), the electrode sections optionally configured for use in a PFA procedure. An insulated or non-conductive area 1714 is disposed between the positive and negative electrodes. Ablation portion 1702 may expand from a collapsed state (not shown) to an expanded state of about 25 mm in diameter, according to some embodiments.

According to some embodiments, when energy is applied from an energy source (not shown), an electrical field will be generated between the positive electrode 1712 and negative electrode 1716, thereby allowing ablation of tissue such as, for example, heart tissue, as discussed herein, located adjacent to and/or between the positive and negative electrodes. According to some embodiments, up to 3,000 volts may be transferred across insulated section 1714, between positive electrode 1712 and negative electrode 1716.

According to some embodiments, device 1700 may be provided with a first conductive ring 1710 at a distal portion of the body 1704, proximal to the ablation portion 1702, and a second conductive ring 1718 at a distal portion of the ablation portion 1702. Energy may be transmitted from a generator or other energy source (not shown) via a wire to conductive rings 1710 and 1718, for transmitting energy to positive electrode 1712 and negative electrode 1716, respectively.

FIG. 17C is a schematic illustration showing the ablation device 1700 with ablation portion 1702 in an expanded state, at least partly within an opening of a pulmonary vein 950 in the heart, according to some exemplary embodiments. The positioning and function of ablation device 1700 may be identical or similar to those of device 900 (FIG. 9F), and may not be discussed further herein.

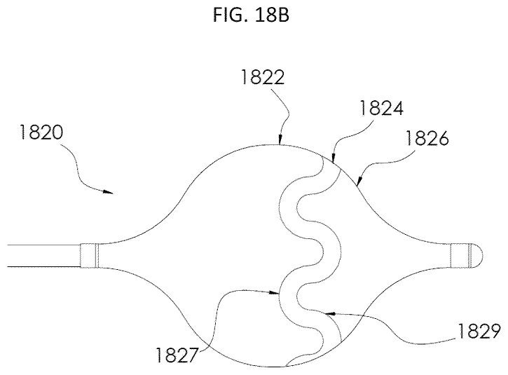

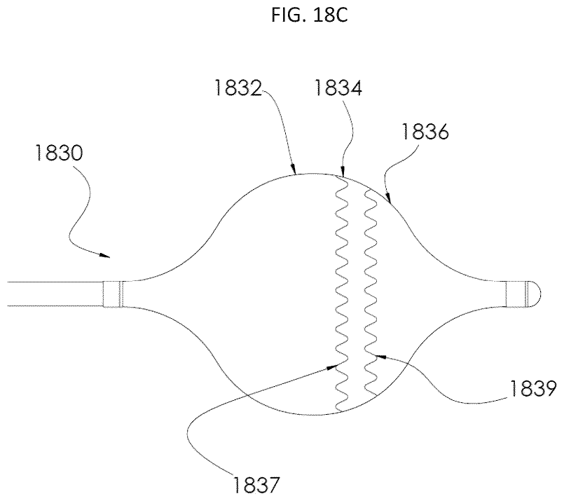

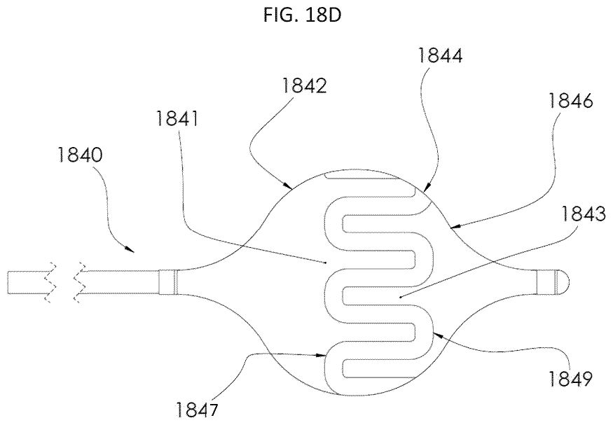

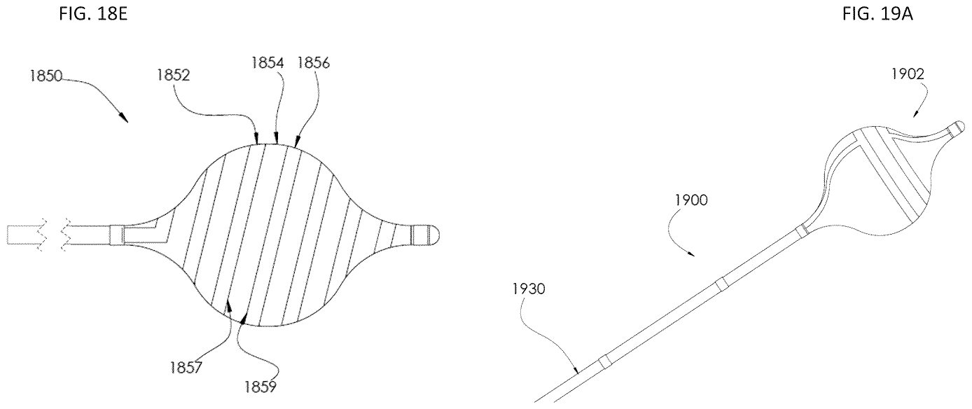

FIG. 18A is a schematic illustration showing an ablation portion 1802, according to some embodiments. Ablation portion 1802 may be identical or similar in much of its structure and function to ablation portion 1702 (FIG. 17A). However, ablation portion 1802 may optionally include at least one radiopaque marker in the form of at least one radiopaque band 1818 on at least one of positive electrode 1812 and negative electrode 1816, optionally adjacent a non-conductive area 1814. The provision of at least one radiopaque marker may assist an electrophysiologist, for example, to visualize the ablation area under x-ray, according to some embodiments.

It may be noted that, due to the shapes of positive electrode 1812, negative electrode 1816, and insulative portion 1814 therebetween, a border 1804 formed between the positive electrode 1812 and the insulative portion 1814 defines a straight line extending around ablation portion 1802, and a border 1806 formed between the negative electrode 1816 and the insulative portion 1814 defines a straight line extending around ablation portion 1802, according to some embodiments. Such a configuration may allow ablation of a circumferential portion of tissue having approximately straight edges, for example, in a blood vessel in which ablation portion 1820 is positioned.

FIGS. 18B-E are schematic illustrations showing the ablation portions of alternative ablation devices, each in an expanded state, according to some additional exemplary embodiments. According to some embodiments, an ablation portion may be provided with a predefined sections acting as positive and negative electrodes, as discussed herein, the predefined sections determining ablation areas having preselected geometric shapes other than those shown in FIGS. 17A-C such as, for example, waves including undulating waves, triangles, and circles. Yet further exemplary ablation portions having various geometrically-shaped positive and negative electrodes or electrode portions may be provided. For example, positive and negative electrode portions separated by an insulative portion extending around the ablation portion may be useful in ablating tissue in a circumferential pattern.

For example, FIG. 18B shows an ablation portion 1820 having a positive electrode 1822 and a negative electrode 1826 having an insulative portion 1824 therebetween, where a border 1827 in the form of an undulating wave extending around ablation portion 1820 is defined between the positive electrode 1822 and the insulative portion 1824, and a border 1829 in the form of an undulating wave extending around ablation portion 1820 is defined between the negative electrode 1826 and the insulative portion 1824, according to some embodiments. This configuration may allow a larger ablation area than that provided by an ablation device having an insulative portion forming a straight line border between positive and negative electrode sections, since the undulating shape may create multiple contact points with the pulmonary vein's funnel and with tissue inside the pulmonary vein. Optionally, this configuration may better facilitate contact with a wall of a PV than an electrode section bordered by a straight line. Optionally, the insulative portion may include undulations, thereby providing larger contact points between the ablation portion and the tissue to be ablated.

FIG. 18C shows an ablation portion 1830 having a positive electrode 1832 and a negative electrode 1836 having an insulative portion 1834 therebetween, where a border 1837 in the form of a zigzag extending around ablation portion 1830 is defined between the positive electrode 1832 and the insulative portion 1834, and a border 1839 in the form of a zigzag extending around ablation portion 1830 is defined between the negative electrode 1836 and the insulative portion 1834, according to some embodiments. This configuration may provide a larger ablation area than that provided by an ablation portion having an insulative section forming a straight line border between positive and negative electrode sections, since the zigzag pattern may create multiple contact points with the pulmonary vein's funnel and with tissue inside the pulmonary vein.

FIG. 18D shows an ablation portion 1840 having a positive electrode 1842 including distally-extending projections 1841 extending along ablation portion 1840 and a negative electrode 1846 having proximally-extending projections 1843 extending along ablation portion 1840, and an insulative portion 1844 therebetween, where a border 1847 in the form of an elongated undulating wave extending around ablation portion 1840 is defined between the positive electrode 1842 and the insulative portion 1844, and a border 1849 in the form of an elongated undulating wave extending around ablation portion 1840 is defined between the negative electrode 1846 and the insulative portion 1844, according to some embodiments. This configuration may provide a larger ablation area, since the ablation occurs between projections. Optionally, a length of the projections can be selected to be longer or shorter, resulting on a larger or smaller portion of the balloon with which to perform the ablation.

FIG. 18E shows an ablation portion 1850 having a positive electrode 1852 and a negative electrode 1856, each extending in a spiral pattern around the ablation portion 1850, wherein each spiral is a circular pattern beginning at a proximal portion of the ablation portion and circling around the ablation portion as it extends distally to a distal portion of the ablation portion. In this manner, each of the positive and negative electrodes form spiral of alternating positive and negative electrode portions separated by an insulative portion 1854 also having a spiral pattern. A border 1857 in the form of a spiral extending around ablation portion 1850 is defined between the positive electrode 1852 and the insulative portion 1854, and a border 1859 in the form of a spiral extending around ablation portion 1850 is defined between the negative electrode 1856 and the insulative portion 1854, according to some embodiments. Optionally, the pitch of the spiral may be preselected. This configuration, due to the two spiral electrode sections positioned on the balloon, allows the entire balloon to be used to ablate the tissue.

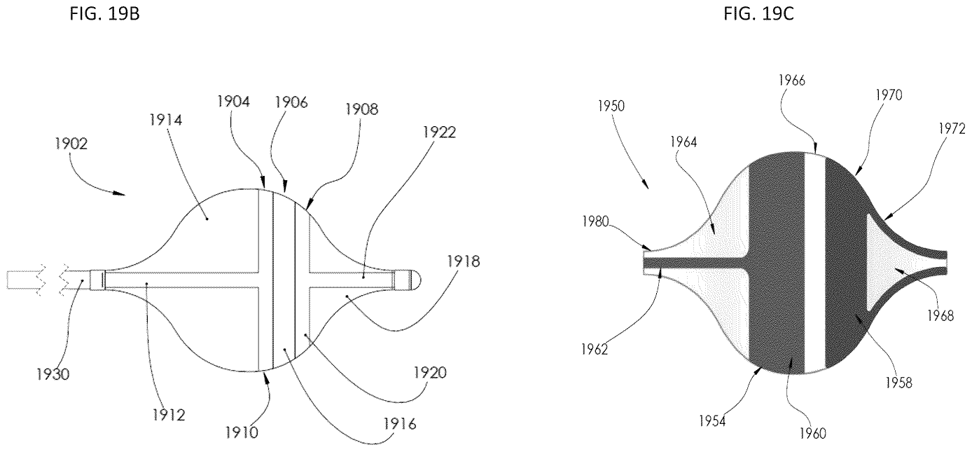

FIG. 19A shows an ablation device 1900 including a body 1930 and an ablation portion 1902 having various components identical or similar to those of device 1700 having ablation portion 1702, according to some embodiments. Components of device 1900 having identical or similar structure and function to components described earlier herein may not be described again herein. Ablation portion 1902 includes a positive electrode 1904 having a circumferential first portion 1910 extending around ablation portion 1902 and a second portion 1912 extending proximally from the circumferential first portion 1910 to body 1930, and a negative electrode 1908 having a circumferential first portion 1920 extending around ablation portion 1902 and a second portion 1922 extending distally from the circumferential first portion 1920, according to some embodiments. A first insulative portion 1916 having a ring shape is defined on ablation portion 1902, between positive electrode 1904 and negative electrode 1908. A second insulative portion 1914 is formed between positive electrode 1904 and body 1930, and a third insulative portion 1918 is formed between negative electrode 1908 and a distal end of ablation portion 1902. This configuration, as compared with that of FIG. 17B, may allow the conductive surface portions of the balloon, i.e., the conductive portions 1910 and 1920, to potentially be reduced. This may reduce costs in manufacturability Additionally, reducing the conductive portions of the balloon may allow an operator, such as a surgeon, to better direct the conductive portions toward narrower portions of tissue to be ablated, thereby potentially increasing safety of the patient.

FIG. 19C shows an ablation portion 1950 of a device having components which may be identical or similar to ablation portion 1902 of device 1900 (FIG. 19A), according to some exemplary embodiments. Components of ablation portion 1950 which are identical or similar in structure and function to those of ablation portion 1902 may not be described again herein. According to some exemplary embodiments, ablation portion 1950 includes a first electrode 1954 such as, for example, a positive electrode, having a circumferential first portion 1960 extending around ablation portion 1950 and a second portion 1962 extending proximally from the circumferential first portion 1960 to body 1980, and a second electrode 1958 such as, for example, a negative electrode, having a circumferential first portion 1970 extending around ablation portion 1950 and a second portion 1972 extending distally from the circumferential first portion 1970, according to some embodiments. According to some exemplary embodiments, a first insulative portion 1966 having a ring shape is defined on ablation portion 1950, between positive electrode 1954 and negative electrode 1958. A second insulative portion 1964 is formed between positive electrode 1954 and body 1980, and a third insulative portion 1968 is formed between negative electrode 1958 and a distal end of ablation portion 1950, according to some exemplary embodiments. This configuration may allow insulative portion 1966 to potentially be reduced, in a manner identical or similar to insulative portion 1916 (FIGS. 19A-B). This may reduce costs in manufacturability, and may increase safety of the patient, according to some exemplary embodiments.

It may be noted that electrode portions 1960 and 1970 may be wider than corresponding electrode portions 1910 and 1920 in the embodiment of FIG. 19B. This may allow for a more efficient ablation device, as there may be more contact provided between the ablation portion 1950 and tissue to be ablated, according to some exemplary embodiments.

With regard to the embodiments of FIGS. 17A-19C, it should be noted that, while these are described as having first and second electrode sections separated by at least one insulative portion, optionally, any of these embodiments may include first and second electrode sections, at least one of which may be partly coated with an insulative material and may have one or more windows through which the electrode is exposed. The provision of one or more windows through which an electrode may be exposed is potentially advantageous in that it may reduce manufacturing costs.

Further, with regard to the embodiments of FIGS. 17A-19C, it should be noted that, the first and second electrode sections may be separated by at least one insulative portion having undulations or other shapes creating longer borders between the first and second electrodes having, thereby potentially allowing more tissue to be ablated.

According to some embodiments such as, for example, those described with regard to FIGS. 17A-19C, a diameter of an expanded ablation portion may be in a range of from about 20-35 mm such as, for example, from about 20-25 mm, from about 25-30 mm, or from about 30-35 mm, which may be suitable for partial insertion into a pulmonary vein funnel, with about 10 mm or more of the balloon remaining within the left atrium, outside the pulmonary vein.

According to some embodiments, the ablation portion of any of the devices described with reference to FIGS. 17A-19C may include a balloon portion formed of any suitable material such as, for example, Pebax® (polyether block amide or PEBA), nylon, silicone, ChronoFlexAL® (any of a family of premium polycarbonate aliphatic biodurable thermoplastic polyurethane elastomers), PET (polyethylene terephthalate), Pellethane® (any of a family of medical-grade thermoplastic polyurethane elastomers), or any combination thereof. A coating forming the electrode sections of the ablation portions of any of the devices described with reference to FIGS. 17A-19C may be any suitable biocompatible conductive material such as, for example, gold, stainless steel, graphite, polycaprolactone, or any other biocompatible conductive material.

With regard to embodiments including a balloon, it should be noted that, according to some embodiments, the balloon may be non-complaint, half compliant or fully complaint, depending on portions inside the funnel of a pulmonary vein or of a pulmonary vein it is desired to ablate. For example, a compliant balloon may facilitate ablating a thin circumferential portion of tissue, while a non-compliant balloon may facilitate ablating a wider circumferential portion of tissue. According to some embodiments, a non-compliant balloon may stretch as it expands, or may expand and collapse without stretching of the balloon material. Optionally, a compliant balloon may be made of a material that is stretchable, such that its material will stretch as it expands from the collapsed state to the expanded state, according to some embodiments. Optionally, the balloon may contact a large area within the funnel of a pulmonary vein and/or inside a pulmonary vein, according to some embodiments.

It may also be noted that, according any of the embodiments discussed herein, use of an ablation device having an ablation portion including a balloon such as, for example, a compliant balloon, may have a potential advantage in that it may adjust better to the anatomy of the tissue of the pulmonary vein/pulmonary vein funnel than a device having electrode wires (with no balloon structure). Additionally, an ablation portion according to any of the embodiments discussed herein which includes a balloon may prevent blood from being trapped inside or between components of the device, thereby potentially preventing a blood clot, according to some embodiments.

With reference to FIGS. 20A-C there is shown an ablation device 2000 having a proximal end 2012 and a distal end 2014, according to some exemplary embodiments. Ablation device 2000 includes a flexible shaft 2002, which extends along longitudinal axis 2006, and an ablation portion 2004 having a framework in the form of a spiral structure 2010 (also referred to as “spiral 2010”) at device distal end 2014, according to some exemplary embodiments. Spiral structure 2010 may be formed of any suitable material and may include, for example, a nitinol core 2020 with an outer insulative covering 2022. Flexible shaft 2002 and spiral structure 2010 may each have a cross-sectional profile having a diameter in a range of from about 1.5-4 mm such as, for example, from about 1.5-2.5 mm, from about 2.5-3.5 mm, or from about 3-4 mm, according to some exemplary embodiments.

It may be noted that spiral structure 2010 may have a particular feature in that no portions of the spiral structure intersect with each other. This particular feature may allow the spiral structure to better contact and/or conform to tissue to be ablated, according to some embodiments.

While ablation device 2000 is shown in an expanded state, it is to be understood that ablation device 2000, in a collapsed, axial state (not shown), may be delivered to an ablation site via a catheter or other deployment device (not shown), as discussed herein, according to some exemplary embodiments. Once released from the catheter or other deployment device, the ablation portion 2004 may self-expand to assume the spiral shape shown, where the coils at the distal portion of the spiral structure 2010 may be narrower than coils at the proximal portion of the spiral structure, according to some exemplary embodiments. While in the embodiment shown the spiral structure 2010 has about 2 coils or revolutions, according to some embodiments, the spiral structure 2010 may have any number of coils or revolutions in a range of from about 0.5 to 5 such as, for example, from about 0.5-1, from about 1-3, and from about 3-5 revolutions, according to some exemplary embodiments. The number of revolutions in the spiral structure 2010 may be selected depending on the area of tissue to be ablated, such as, for example, a circumferential portion of tissue.

The spiral structure 2010 may have a radius R, at the widest portion thereof, in a range of from about 4-18 mm such as, for example, from about 4-6 mm, from about about 6-8 mm, from about 8-10 mm, from about 10-12 mm, from about 12-14 mm, from about 14-16 mm, of from about 16-18 mm, according to some exemplary embodiments. The spiral structure 2010 may have a pitch P between adjacent coils of the spiral structure in a range of from about 3-15 mm such as, for example, from about 3-5 mm, from about 5-7 mm, from about 7-9 mm, from about 9-11 mm, from about 11-13 mm, or from about 1315 mm, according to some exemplary embodiments.

While the framework of the ablation portion is shown in FIGS. 20A-C as being in the form of a spiral structure 2010, alternatively, the framework may have a cylindrical configuration or a zigzag configuration, optionally defined in a single plane, according to some exemplary embodiments.

At least a first electrode wire 2016, having a first polarity, and a second electrode wire 2018, having a second polarity, may be spirally wound around spiral structure 2010, according to some exemplary embodiments. First and second electrode wires 2016 and 2018 may each be formed of any suitable electrically conductive material, as discussed herein; may have any suitable cross-sectional profile such as, for example, circular or elliptical; and the cross-sectional profile of each of electrode wires 2016 and 2018 may have a diameter in a range of from about 0.1-0.5 mm such as, for example, from about 0.1-0.2 mm, from about 0.2-0.3 mm, from about 0.3-0.4 mm, or from about 0.4-0.5 mm, according to some exemplary embodiments. Optionally, according to some exemplary embodiments, electrode wires 2016 and 2018 each has a cross-sectional profile having no sharp corners, to avoid damage to tissue with which they may come into contact. Optionally, instead of first and second electrode wires 2016 and 2018, electrode ribbons may be wound around spiral structure 2010, according to some embodiments.

It may be noted that the windings of first electrode wire 2016 and second electrode wire 2018 may be specifically configured such that the spacing S (FIG. 20C) between the first electrode wire and the second electrode wire are consistent along the spiral structure 2010, according to some exemplary embodiments. The spacing S between windings of the first and second electrode wires 2016 and 2018 may be in a range of from about 1-5 mm such as, for example, from about 1-1.5 mm, from about 1.5-2 mm, from about 2-2.5 mm, from about 2.5-3 mm, from about 3-3.5 mm, from about 3.5-4 mm, from about 4-4.5 mm, or from about 4.5-5 mm, according to some exemplary embodiments.

In order to maintain the spacing between electrode wires 2016 and 2018, and to prevent movement of the electrode wires 2016 and 2018 relative to the spiral structure 2010 and to each other, the electrode wires may optionally be glued to the spiral structure 2010, according to some exemplary embodiments. Alternatively, the electrode wires 2016 and 2018 may be partially embedded in the material of the spiral structure 2010, for example, by applying an electrical current to embed the electrode wires 2016 and 2018 partially within the material of the spiral structure 2010, by heating in an oven, or by heating during a heat shrink procedure, according to some exemplary embodiments.

According to some exemplary embodiments, pulse field ablation may be performed for tissue in contact with ablation portion 2004, as an electric pulse may be created between electrode wires 2016 and 2018, as discussed herein. It may be noted that the spiral structure 2010 may be particularly suitable for ablating tissue, for example, anatomical structures such as a pulmonary vein funnel and the pulmonary vein itself, due to its spiral shape having narrower coils at its distal portion and due to its flexible structure, according to some exemplary embodiments. Additionally, due to the spiral structure of the ablation portion 2004, an ablation procedure can potentially treat a large area around the pulmonary vein funnel or inside the pulmonary vein, according to some exemplary embodiments.

Optionally, ablation portion 2004 may include an outer dielectric cover 2024, extending along the spiral structure 2010, the dielectric cover preventing or reducing the intensity of an electric field passing from the ablation portion 2004 to tissue in contact with the ablation portion, according to some exemplary embodiments. Additionally, dielectric cover 2024 may prevent any sharp or unsmooth edges of the ablation portion 2004 from damaging tissue with which it may come into contact, according to some exemplary embodiments.

Optionally, the ablation portion 2004 may be provided with at least one additional electrode wire such as, for example, a third electrode wire (not shown), wound around the spiral structure 2010, in a manner identical or similar to that of electrode wires 2016 and 2018, the third electrode wire evenly spaced from each of said first and second electrode wires 2016 and 2018, according to some exemplary embodiments.

Optionally, the ablation portion 2004 may be additionally provided with a fourth electrode wire (not shown) wound around the spiral structure 2010, in a manner identical or similar to that of electrode wires 2016 and 2018 and the third electrode wire, the fourth electrode wire evenly spaced from each of the first and second electrode wires 2016 and 2018 and from the third electrode wire, according to some exemplary embodiments.

The provision of at least one additional electrode wire may allow an ablation device to have a plurality of selectable ablation modes, according to some exemplary embodiments. For example, an ablation device may be provided with three evenly spaced electrode wires, such as wires A and B each having a first polarity, and wire C having a second polarity, where adjacent wires are positioned 1 mm apart, according to some exemplary embodiments. The device may then have a first ablation mode, in which electrode wires B and C (which are 1 mm apart) are activated; and a second ablation mode, in which electrode wires A and C (which are 2 mm apart) are activated, thereby producing a larger pulse field than that produced by electrode wires B and C, and a correspondingly deeper ablation effect, according to some exemplary embodiments. In another exemplary embodiment, an ablation device may be provided with four evenly spaced electrode wires, such as wires A, B, and C, each having a first polarity, and wire D having a second polarity, where adjacent wires are positioned 1 mm apart. The device may then have a first ablation mode, in which electrode wires C and D (which are 1 mm apart) are activated; a second ablation mode, in which electrode wires B and D (which are 2 mm apart) are activated, thereby producing a larger pulse field than that produced by electrode wires C and D, and a correspondingly deeper ablation effect; and a third ablation mode, in which electrode wires A and D (which are 3 mm apart) are activated, thereby producing an even larger pulse field than that produced by electrode wires B and D, and a correspondingly deeper ablation effect, according to some exemplary embodiments. It will be appreciated by persons skilled in the art that, optionally, any preselected number of electrode wires may be provided on the ablation device, thereby providing the device with a preselected number of ablation modes, as discussed herein, according to some exemplary embodiments.

It should be noted that the embodiment of FIG. 20C includes a spiral structure 2010 having electrode wires 2016 and 2018 wound therearound, wherein PFA may be induced by the electrode wires 2016 and 2018. According to some alternative embodiments, PFA may be performed by an electrode forming a core of the spiral such as, for example, nitinol core 2020, together with one of electrode wires 2016 and 2018, provided that there is no insulative covering 2022 present and that the electrode wire being used is maintained at a distance from nitinol core 2010, such that there is no contact between nitinol core 2020 and the electrode wire being employed in the PFA. Alternatively, the nitinol core 2020 may be provided with an insulative covering having apertures corresponding to windings of the electrode wire being employed, the insulative covering preventing contact between the nitinol core and the electrode wire being employed, and PFA may be performed utilizing the nitinol core 2020 and the electrode wire, via the apertures in the insulative covering.

Optionally, the device of any of the embodiments shown or discussed herein such as, for example device 2000 may be provided with a radiopaque marker on at least one of the first and second electrodes or electrode sections, for facilitating the visualization of the location of the electrodes or electrode sections under x-ray, as discussed herein.

It is expected that during the life of a patent maturing from this application many relevant long or elongated electrodes, or expandable structures will be developed; the scope of the term long or elongated electrode and/or the term expandable ablation portion is intended to include all such new technologies a priori.

The terms “comprises”, “comprising”, “includes”, “including”, “has”, “having” and their conjugates mean “including but not limited to”.

The term “consisting of”means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound”may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An ablation catheter device, comprising:
an elongated flexible body having a long axis, a proximal end and a distal end;
an expandable therapeutic portion coupled to said elongated flexible body and located between said proximal end and said distal end of said elongated flexible body, wherein said expandable therapeutic portion is configured to move between a collapsed state and an expanded state; and
two or more parallel elongated electrodes extending axially along said expandable therapeutic portion, wherein said expandable therapeutic portion allows blood to flow through said expandable therapeutic portion.

2. A device according to claim 1, wherein said expandable therapeutic portion comprises a braided mesh structure configured to move between said collapsed state and said expanded state, and wherein said two or more elongated electrodes surround said braided mesh structure; and
wherein said two or more elongated electrodes are twisted around or interwoven with the braided mesh structure.

3. A device according to claim 1, wherein said expandable therapeutic portion comprises a braided mesh structure configured to move between said collapsed state and said expanded state, and wherein said two or more elongated electrodes surround said braided mesh structure; and
wherein said two or more electrodes have a helical or zigzag pattern around said braided mesh structure.

4. A device according to claim 1, wherein said two or more electrodes include a plurality of first electrodes having a first polarity and a plurality of second electrodes having a second polarity;
wherein at least one of:
said body distal end includes a conductive distal plate; and wherein said plurality of first electrodes are electrically connected to said conductive distal plate and said plurality of second electrodes are electrically isolated from said conductive distal plate; and
said body proximal end includes a conductive proximal plate; and wherein said plurality of second electrodes are electrically connected to said conductive proximal plate and said plurality of first electrodes are electrically isolated from said conductive proximal plate, wherein one of said first electrodes extends proximally through an aperture in said conductive proximal plate and is connected to a conductive wire that extends proximally toward said body proximal end, and wherein one of said second electrodes extends proximally through said conductive proximal plate and is connected to said conductive wire.

5. A device according to claim 1, wherein said expandable therapeutic portion comprises an enclosed inflatable structure configured to move between a deflated state in said collapsed state, and an inflated expanded state in said expanded state, and wherein said two or more elongated electrodes are coupled to an outer surface of said enclosed inflatable structure and surround said enclosed inflatable structure;
wherein said therapeutic portion comprises segments of electrically insulating coating partially covering the electrodes and the outer surface of said enclosed inflatable structure while keeping exposed sections shaped as rings surrounding said therapeutic portion that include exposed portions of adjacent electrodes.

6. A device according to claim 1, including at least one tensioner for at least one of:
maintaining said elongated electrodes at a preselected distance apart from each other;
maintaining said elongated electrodes at a preselected distance away from a longitudinal axis of said expandable therapeutic portion; and
preventing inadvertent deformation of said elongated electrodes.

7. A device according to claim 1, wherein each said elongated electrode includes a distal end portion extending radially inwardly and proximally.

8. A device according to claim 1, wherein said expandable therapeutic portion includes a plurality of axially extending wires extending distally from said body when in said collapsed state, said plurality of axially extending wires including at least a first electrode wire extending axially from said body and having a first polarity and at least a second electrode wire extending axially from said body and having a second polarity, wherein at least said first electrode wire is separated from said second electrode wire by a preselected distance; and
wherein said plurality of axially extending wires each includes a proximal wire portion and a distal wire portion, wherein at least one of said proximal wire portions and said distal wire portions includes an isolating material configured to maintain adjacent ones of said plurality of axially extending wires at a preselected distance apart from each other.

9. A device according to claim 8, wherein said isolating material is one of:
a coating on said plurality of axially extending wires;
a plating on said plurality of axially extending wires;
a cover radially external to said plurality of axially extending wires; and
a first cover radially external to said plurality of axially extending wires and a second cover radially internal to said plurality of axially extending wires.

10. A device according to claim 8, wherein said isolating material is one of:
a cover radially external to said plurality of axially extending wires, said cover including through holes; and
a first cover radially external to said plurality of axially extending wires and a second cover radially internal to said plurality of axially extending wires, wherein said first and second cover include through holes.

11. A device according to claim 8, wherein said isolating material is at least one of biocompatible, non-thrombogenic, and microporous.

12. A device according to claim 8, wherein said axially extending wires include said isolating material at least one of:
on at most 15% of a most distal portion thereof; and
on at most 50% of a most proximal portion thereof.

13. An ablation catheter device according to claim 1, wherein said expandable therapeutic portion comprises at least one central flexible shaft configured to move between said collapsed state and said expanded state; and
wherein said two or more elongated electrodes are coupled to said expandable therapeutic portion, and wherein said two or more elongated electrodes are twisted around said at least one central flexible shaft.

14. A device according to claim 13, wherein said two or more elongated electrodes are coated with a dielectric coating.

15. A device according to claim 13, wherein said at least one central flexible shaft is formed from an electrically insulating material or is coated with an electrically insulating material.

16. A device according to claim 13, wherein said at least one central flexible shaft is formed from a shape memory alloy.

17. A system for ablating tissue comprising the ablation catheter device according to claim 1, wherein two or more elongated electrodes surround said expandable therapeutic portion; and
wherein said system includes a control unit coupled to said ablation catheter device, said control unit comprising:
a memory configured for storing parameter values of an electric field suitable for delivery of pulse field ablation (PFA) to a body tissue, wherein said parameter values comprise at least one of voltage, frequency, pulse duration and number of pulses;
a pulse generator electrically connected to said two or more electrodes;
a control circuitry, wherein said control circuitry is configured to signal said pulse generator to generate said electric field according to said parameter values stored in said memory and to deliver said generated electric field to said two or more electrodes;
wherein said ablation catheter device comprises at least one additional electrode located distally to said expandable therapeutic portion, and wherein said control circuitry is configured to signal said pulse generator to generate and deliver an electric field via said at least one additional electrode to a tissue with parameter values suitable for pacing the tissue.

18. A device according to claim 1, wherein said therapeutic portion includes an axially extending portion, wherein said at least first and second electrodes are wound around said axially extending portion, and wherein said at least first and second electrodes are evenly spaced from each other.

19. A device according to claim 1, wherein said therapeutic portion includes an axially extending portion;
wherein said device includes a dielectric cover positioned on at least a portion of said axially extending portion, said dielectric cover configured to prevent or reduce the intensity of an electric field passing from said therapeutic portion to tissue in contact with said therapeutic portion.

20. A device according to claim 18, wherein said therapeutic portion includes at least a third electrode wound around said axially extending portion and evenly spaced from each of said first and second electrodes.

21. A device according to claim 20, wherein said ablation catheter device is configured to operate in a plurality of ablation modes, wherein in each one of said plurality of ablation modes said ablation catheter device is configured to deliver pulse field ablation (PFA) of a preselected protocol to a body tissue; wherein at least one of:
in a first ablation mode, said device is configured to use said first and second electrodes to deliver the PFA;
in a second ablation mode, said device is configured to use said first and third electrodes to deliver the PFA; and
in a third ablation mode, said device is configured to use said second and third electrodes to deliver the PFA.

22. A method for delivery of a pulse field ablation (PFA), comprising:
inserting an ablation device at least partly into an ostium of a pulmonary vein in a left atrium, wherein the ablation device includes a therapeutic portion having at least two elongated electrodes, wherein the therapeutic portion has a collapsed state in which the elongated electrodes are exposed;
expanding the therapeutic portion;
delivering at least one pulse of an electric field between said at least two electrodes and to a wall of the pulmonary vein with ablation parameter values suitable for pulse field ablation; and
generating a non-thermal ablation region in at least one of tissue of the pulmonary vein and heart tissue which surrounds a lumen of the pulmonary vein by said delivered at least one electric field pulse, wherein said non-thermal ablation region prevents transmission of electrical signals in the heart tissue via said non-thermal ablation region.

23. A method according to claim 22, wherein said non-thermal ablation region is substantially uniform and continuous.

24. A method according to claim 22, comprising adjusting, prior to said delivering, a pitch or a distance between said at least two elongated electrodes according to at least one of location of said at least two electrodes within the pulmonary vein ostium, a shape and/or size of a target ablation region, and a target ablation depth.
